(19) United States
(12) Patent Application Publication
(10) Pub. No.: US 2023/0121588 A1
(43) Pub. Date: Apr. 20, 2023

(54) PARTICLE SETTLING DEVICES INSIDE BIOREACTORS

(71) Applicant: SUDHIN BIOPHARMA, Superior, CO (US)

(72) Inventors: Dhinakar S. Kompala, Superior, CO (US); Braden L. Smith, Lafayette, CO (US)

(73) Assignee: SUDHIN BIOPHARMA, Superior, CO (US)

(21) Appl. No.: 17/968,479

(22) Filed: Oct. 18, 2022

Related U.S. Application Data
(60) Provisional application No. 63/256,924, filed on Oct. 18, 2021.

(51) Int. Cl. C12M 1/00; C12M 1/26; C12M 1/34

(52) U.S. Cl. CPC C12M 29/10; C12M 27/20; C12M 33/22; C12M 41/00

(57) ABSTRACT

Settling or settler devices for separating particles from a bulk fluid with applications in numerous fields. The devices include inclined sliders with an arcuate cross-section arranged in a space within a bioreactor. These devices are useful for separating small (millimeter or micron sized) particles from a bulk fluid with applications in numerous fields, such as biological (microbial, mammalian, plant, insect or algal) cell cultures, solid catalyst particle separation from a liquid or gas and wastewater treatment, organic and aqueous phase emulsion separations, or the like.

### CROSS REFERENCE TO RELATED APPLICATIONS

This U.S. Non-Provisional Pat. Application claims the benefit of priority from U.S. Provisional Pat. Application Serial No. 63/256,924, filed Oct. 18, 2021, the disclosure of which is incorporated herein by reference in its entirety.

This application is related to: U.S. Pat. Application 17/205,858, filed Mar. 18, 2021, and PCT Application No. PCT/US2021/023006 having an international filing date of Mar. 18, 2021, and which designated the United States, which each claim the benefit of priority under 35 U.S.C. §119(e) to U.S. Provisional Pat. Application Serial No. 62/991,976, filed Mar. 19, 2020; U.S. Pat. Application 16/375,683, filed Apr. 4, 2019, now U.S. Pat. No. 10,576,399, which claims the benefit of priority under 35 U.S.C. § 119(e) to U.S. Provisional Pat. Application Serial No. 62/659,295 filed Apr. 18, 2018; U.S. Pat. Application No. 15/586,902, filed May 4, 2017, now U.S. Pat. No. 10,596,492, which application is a continuation in part of U.S. Pat. Application No. 15/324,062, filed Jan. 5, 2017, which is a national stage application under 35 U.S.C. §371 of PCT Application No. PCT/US2015/039723 having an international filing date of Jul. 9, 2015, which designated the United States, which PCT application claims the benefit of U.S. Provisional Pat. Application No. 62/022,276, filed Jul. 9, 2014; PCT Application No. PCT/US2015/063195 having an international filing date of Dec. 1, 2015, which designated the United States, which PCT application claims the benefit of U.S. Provisional Pat. Application No. 62/086,122, filed Dec. 1, 2014; U.S. Provisional Pat. Application No. 62/332,546, filed May 6, 2016; U.S. Provisional Pat. Application No. 62/459,509, filed Feb. 15, 2017; and U.S. Provisional Pat. Application No. 62/037,513, filed Aug. 14, 2014; the entire disclosures of which are each incorporated herein by reference in their entirety.

### FIELD

This disclosure provides cell or particle settling or settler devices with enhanced settling on multilayered inclined surfaces. This devices of the present disclosure have applications in numerous bioprocessing or biomanufacturing applications, including: (i) retention of live and productive microbial or mammalian cells inside the bioreactor while the secreted protein products are harvested continuously along with a significantly clarified cell culture broth containing very few live cells; (ii) selective removal of smaller, slower-settling dead cells and cell debris during ex vivo cell expansion cultures to enhance the viability of cell therapy products such as mesenchymal stem cells (MSC), induced pluripotent stem cells (iPSC), chimeric antigen receptor-T lymphocytes (CAR-T cells), etc.; (iii) removal of spent cell culture medium during regular media exchange protocols in ex vivo expansion of stem cell clusters, cell aggregates, organoids such as beta islet cells, adherent cells grown on microcarrier beads, etc.; and (iv) harvesting adherent cells detached from microcarrier beads after growing up to confluency, while retaining the denuded microcarrier beads inside the bioreactor.

### DESCRIPTION OF RELATED ART

Of all the above-mentioned fields of application for settling or settler devices, the more immediately applicable well-established field is the production of secreted biological proteins, polypeptides or hormones secreted from suspension cultures of recombinant microbial or mammalian cells. Most common methods of producing secreted biological proteins in recombinant mammalian cells rely on fed-batch cultures, wherein cells are grown to high cell densities and the constitutively expressed secreted proteins accumulate until the fed-batch culture is terminated due to accumulation of metabolic waste by-products causing cell death. With recombinant microbial cells (e.g., yeast cells) which are capable of secreting heterologous expressed proteins, fed-batch bioreactors are commonly used to achieve high cell density and then the cells are typically exposed to an induction medium or inducer to trigger the production of proteins. If the desired proteins are secreted out of either mammalian or microbial cells, it is more advantageous to switch from a fed-batch culture to a continuous perfusion culture, which can maintain high cell density and high productivity over a much longer duration of culture. During such continuous perfusion cultures, live and productive cells can be retained or recycled back to the bioreactor while the secreted proteins are continuously harvested from the bioreactor for downstream purification processes.

Some key advantages of continuous perfusion cultures over fed-batch cultures are: (i) the secreted protein products are continuously removed from the bioreactor, without subjecting these products to potential degradation by proteolytic and/or glycolytic enzymes released into the culture medium from dead cells; (ii) live and productive cells are retained or recycled back to achieve high cell densities in continuous perfusion bioreactors, where they continue to produce valuable proteins inside the controlled bioreactor environment for much longer culture duration, rather than being removed from the bioreactor at the end of each fed-batch culture; (iii) the perfusion bioreactor environment can be maintained at much closer to a steady state (maintaining a constant product quality by design) with fresh nutrient media being continuously fed and metabolic toxic waste by-products removed along with the harvest, unlike the dynamically changing concentrations of nutrients and waste products in a fed-batch culture; and (iv) with a subset of cell retention devices, smaller dead or dying cells can be selectively removed from the perfusion bioreactor before these cells lyse and release their intracellular enzymes, thereby maintaining a high viability fraction of cells and high quality of the secreted protein products as they are harvested. It is noted perfusion bioreactors are devices that employ continuous cell culturing methods in which live and productive cells are either retained in the bioreactor or recycled back into the bioreactor, while the secreted products are removed continuously along with the spent media and, in select instances, dead cells and cell debris.

Many cell retention devices have been developed in the mammalian cell culture industry, such as the internal spin filter devices (Himmelfarb et al., Science 164: 555-557, 1969), external filtration modules (Brennan et al., Biotechnol. Techniques, 1 (3): 169-174, 1987), hollow fiber modules (Knazek et al., Science, 178: 65-67, 1972), gravitational settling in a cyclone (Kitano et al., Appli. Microbiol. Biotechnol. 24, 282-286, 1986), inclined settlers (Batt et al., Biotechnology Progress, 6: 458-464, 1990), continuous centrifugation (Johnson et al., Biotechnology Progress, 12, 855-864, 1999), and acoustic filtering (Gorenflo et al., Biotechnology Progress, 19, 30-36, 2003). While most of these devices adequately retain all mammalian cells from the harvest, these devices are unable to separate dead cells from the live cells desired in the bioreactor. All filtration devices get clogged over the extended culture duration of mammalian cell perfusion bioreactors (typically in 2 to 4 weeks) and require a continuous cell bleed stream to remove the accumulating dead cells along with the productive live cells. The cyclones were found to be incapable of producing enough centrifugal force for sufficient cell separation at the device sizes and harvest flow rates used in the mammalian cell culture experiments (Kitano et al., 1986) and mammalian cells are seriously damaged at higher flow rates (and centrifugal forces) necessary for efficient cell separation (Elsayed, et al., Eng. Life Sci., 6: 347-354, 2006). Continuous centrifuges also subject mammalian cells to high shear forces. Acoustic devices are plagued with excessive heat generation due to the generation of ultrasound waves, which becomes a larger problem to dissipate at bigger scales.

Among all the cell retention devices available today, only the inclined settlers (Batt et al., 1990, and Searles et al., Biotechnology Progress, 10: 198-206, 1994) enable selective removal of smaller dead cells and cell debris in the overflow or harvest stream, while bigger live and productive mammalian cells are continually being recycled via the underflow back to the perfusion bioreactor. Therefore, it is feasible to continue the perfusion bioreactor operation indefinitely at high viability and high cell densities while the protein product is continuously harvested from the top of inclined settler. The inclined settler has previously been scaled up as multi-plate or lamellar settlers (Probstein, R. F.; Yung, D. U.S. Pat. 4151084, April 1979) and used extensively in several large-scale industrial processes such as wastewater treatment, potable water clarification, metal finishing, mining, and catalyst recycling (e.g., Odueyngbo et al., U.S. Pat. Application 2004/0171702 A1, September 2004), etc. Citing our first demonstration of a single plate inclined settler (Batt et al., 1990) to enhance productivity of secreted proteins in mammalian cell culture applications, a multi-plate or lamellar settler device has been patented for the scale up of inclined settlers for use in hybridoma cell culture (Thompson and Wilson, U.S. Pat. No. 5,817,505, October 1998). Such lamellar inclined settler devices have been used to operate continuous perfusion bioreactors at high bioreactor productivity (due to high cell density) and high viability (>90%) for long durations (e.g., several months without any need to terminate the perfusion culture). Other variations of inclined settling devices, such as helically wound closed flexible tubes (Kauling, et al., U.S. Pat. Application Publication No. 2011/0097800 A1) and nested-cone separators (Thilly, PCT Application No. WO199106627) have not been sufficiently well developed, nor scaled up for wider applications.

Unlike the earlier successfully scaled-up designs of inclined settlers, as the stacked or lamellar rectilinear designs and recently patented stacked conical and cylindrical designs, and attached externally to a bioreactor (Kompala, US 10576399B2, US10596492B2), our present invention discloses a novel spiral slide settling device design, which can be inserted or incorporated inside any suspension bioreactor. One key advantage of installing such spiral slides inside a suspension bioreactor with its own pH and Dissolved Oxygen controlled environment is that the cells settling on spiral slides are inside the controlled growth environment of the bioreactor, eliminating a major concern with external modular settlers without such environmental controls.

Thus, a particle settling device that can leverage centrifugal forces and gravitational forces on particles in liquid suspension in a relatively small space within a bioreactor is desired.

### SUMMARY

This disclosure provides novel devices and methods of retaining cells or particles inside a suspension bioreactor by incorporating or inserting a newly designed version of inclined sliders inside any suspension bioreactor. Numerous, layered inclined sliders or plates enhance the settling efficiency of the particles from the bulk fluid moving either downward or upward inside an assembly in which the liquid volume moves progressively around the periphery of the settling surfaces. The internal cell retention devices are useful for increasing the live and productive cell numbers or concentrations inside bioreactors, while the valuable protein or other products secreted by the cells into the culture medium are continuously removed from the bioreactor in a perfusion process. Alternately, these cell retention devices are useful for retaining live cells, cell clusters, or organoids growing ex vivo inside the bioreactors while dead cells and cell debris are continuously removed from the bioreactors. Further, these retention devices are useful for retaining microcarrier beads with attached adherent cells growing in suspension cultures, while fresh cell culture medium is perfused or fed into the bioreactor and a significantly clarified or relatively cell-free or microcarrier-free spent medium is removed continuously from the bioreactor.

The settler devices (or settling devices) of this disclosure may include a housing that encloses a series of inclined slide settlers (or sliders) positioned inside the housing. The sliders of this embodiment are supported at a distance (or channel height and width) between the successive sliders in the series. The sliders may be coupled to the housing or an inner structure at a desired distance (e.g., the desired channel height and width) apart.

The settler devices of this disclosure may include a housing enclosing:
1) a first series of two or more inclined sliders within a cavity or annular space defined by the housing and a first inner structure, and,
2) at least one optional second series of two or more inclined sliders inside an inner cavity or inner annular space defined within a second inner structure with a lesser diameter than the first inner structure.

The inclined sliders (in both the first and optional second series of two or more inclined sliders) may be respectively coupled to the housing and/or first inner structure, or the first inner structure and/or the at least a second inner structure, and are preferably coupled at a substantially constant distance and are formed at a generally equal size to hold each successive inclined settler at about an equal spacing between all of the inclined sliders. There is preferably a substantially constant spacing between each successive inclined slider. The vertical spacing between successive inclined sliders may be varied between about 1 millimeter (mm) to about 5 mm, or between 4 microns to 500 microns. Preferably, in most embodiments the inclined sliders (in both the first and optional second series of two or more inclined sliders) are not coupled to adjacent sliders but instead directly to the housing and/or first inner structure, or the first inner structure and/or the at least a second inner structure. Coupling inclined sliders to adjacent sliders may represent an impediment to settled particles or cells sliding down the surface of the inclined sliders. This arrangement of settling surfaces provided by the inclined sliders is particularly useful for separation applications in which the particle settling device is in need of a compact design to be fit inside a bioreactor.

This arrangement of the first and optional second series of two or more inclined sliders significantly enhances the settling efficiency of particles from a bulk fluid as the bulk fluid moves through the settling device. As outlet fluid flow is controlled and the bulk liquid, including particles such as cells, backfills slowly up the inclined settlers, solid microcarrier particles or semi-solid single cells or cellular aggregates or organoids will settle down on the sliders.

These devices can be scaled up or down to suit the separation needs of different industries or applications or sizes as the separation surface is scaled up or down volumetrically in three dimensions, compared to the more typical one- or two-dimensional scaling of previous settling devices. Scaling up of the devices of this disclosure can be performed simply by increasing the diameter of the housing (and correspondingly increasing the width of the inclined settlers or number of rings of inclined sliders inside) and/or increasing the height of the housing (which increases the height of the inclined sliders in either one or both of the first and at least the second series of inclined sliders). The effective projected area for cell settling increases proportionally to increase of the squared diameter of the housing and increases proportionally to the increase of the height of internal cylinders. The effective settling area of the compact settling devices of this disclosure scales up proportionally to the increase of the housing diameter square (assuming the height of the inclined sliders are also increased proportionally) or equivalently, to the volume of housing. This three dimensional or volumetric scale-up of the effective settling area makes the settling device of this disclosure much more compact compared to previous inclined settler devices.

The settling surfaces may be is convex or concave such that a cross-section of the inclined slider defines an arcuate line, where the particular or cells are guided into a groove by raised sides. For example, the settling surfaces may have a single arcuate shape or multiple arcuate shapes separated by seams. The arcuate shape or shapes may each include a groove in a central location between the interior and exterior surfaces of the housing and/or first inner structure, or the first inner structure and/or the at least a second inner structure to guide the particles or cells, or alternatively positioned closer to either the interior and exterior surfaces of the housing and/or first inner structure, or the first inner structure and/or the at least a second inner structure to guide the particles or cells. Optionally, the settling surfaces may alternatively be planar or flat, where the settling device relies on the interior and exterior surfaces of the housing and/or first inner structure, or the first inner structure and/or the at least a second inner structure to guide the particles or cells.

The angle of inclination for the settling surfaces may or may not be constant, ranging between about 15 degrees to about 75 degrees from the vertical (e.g., as defined by a longitudinal axis of the housing). For use with stickier particles (typically mammalian cells), the angle of inclination may be closer to the vertical (i.e., around 15 degrees from vertical). For use with non-sticky solid catalyst particles, the angle of inclination can be further from vertical (for example, around 75 degrees from vertical). In some embodiments, the sliders have an arcuate longitudinal cross section such that the angle of inclination varies with respect to a longitudinal axis from between about 5 degrees to about 85 degrees, or about 15 degrees to about 75 degrees.

The particle settling devices of this disclosure may include a housing and at least one inner structure or vertical tube disposed inside the housing. For example, the number of vertical tubes within the settler device may be between about 2 and about 50.

All of the settler devices of this disclosure may include a closure or lid over at least a portion of the housing at a first end or a second end of the housing opposite the first end. In all of these embodiments, the closure or lid may also include ports for removing liquids and/or entering liquids into the settler device. The additional ports in the housing and/or the closure or lid are in fluidic communication with the outside and the inside of the housing to facilitate the passage of liquids into and/or out of the housing of the settler device, and in each instance of such ports or inlets/outlets, these passage ways into and out of the housing may include valves or other mechanisms that can be opened or closed to stop or restrict the flow of liquids into or out of the settler devices of this disclosure. There is at least one additional opening in the housing substantially opposite the first opening. The settler device may include a closure over at least a portion of the housing at an end of the housing opposite the first opening. At least one additional opening in the housing may be configured to open from a side of the housing tangential to at least one inner structure, in fluidic communication with the outside and the inside of the housing. A fluid harvest outlet may be formed in a closure, in fluidic communication with the outside and the inside of the housing.

One aspect of the present disclosure is a settling device operable for use in the production of cell therapy products, secreted biological proteins, polypeptides or hormones, vaccines, viral vectors, or gene therapy products. The settling device comprises: (1) a housing with (i) a lower end, (ii) an upper end, and (iii) an interior surface or wall; (2) at least one inner structure installed in the housing with: (i) a lower end, (ii) an upper end, and (iii) an exterior surface or wall; (3) at least one annular space defined by the interior surface of the housing and the exterior surface of the at least one inner structure; (4) multiple inclined sliders provided within the annular space, each inclined slider including (i) a body with an arcuate surface having a groove and sides spaced from a first adjacent inclined slider, and at least one of (ii) an edge coupled to the interior surface of the housing and/or (iii) an edge coupled to the exterior surface of the at least one inner structure, and (iv) a second arcuate surface spaced from a second adjacent inclined slider; (5) an lid or closure connected to a first end of the housing; and (6) an outer conduit or port extending from the lid or closure and optionally extending downwardly in the annular space between the interior surface of the housing and the exterior surface of the at least one inner structure.

A port or outer conduit may be approximately concentrically aligned with the longitudinal axis of the housing. Where there are multiple outer conduits, at least some of the multiple ports may be aligned with concentrically aligned with the longitudinal axis of the housing, or aligned with a separate shared longitudinal axis. The outer conduit may comprise a lumen and/or an orifice to withdraw fluid from the settling device. When present, the orifice is positioned between the upper and lower ends of the cylindrical portion to withdraw fluid from a predetermined level within the settling device.

Additionally, or alternatively, a sensor may be associated with the port or outer conduit to measure a condition within the settling device. In one embodiment, the sensor is operable to measure at least one of pH, dissolved oxygen (DO), dissolved CO₂, glucose, lactate, glutamine, ammonia, and temperature. The sensor may be positioned between the upper and lower ends of the housing. In one embodiment, the sensor is a fluorescent probe. A reader (or meter) to receive light from the fluorescent probe may be positioned in the outer conduit. Optionally, the outer conduit is transparent or translucent such that light from the fluorescent probe can pass therethrough. The reader can transmit data from the fluorescent probe to a control system. In one embodiment, the reader includes an optical fiber or a wire to transmit data to the control system. Additionally, or alternatively, the reader may use a wireless means to transmit the data to the control system.

Optionally, the settler device includes multiple ports or outer conduits extending into the annular space. In one embodiment, a first one of the outer conduits has a first length and a second one of the outer conduits has a second length that is different than the first length. In this manner, the first outer conduit can sample a condition or withdraw fluid at a first height of the housing and a second outer conduit can sample a condition or withdraw fluid at a second height of the housing that is different than the first height.

In one embodiment the lid or top closure or upper portion has a shape that is conical. The conical upper portion includes a first end and a second end. The first end has a first diameter and the second end has a second diameter that is larger than the first diameter. In one embodiment, the first end is oriented toward the bottom closure or lower conical portion.

The settling device optionally includes a second port or conduit extending from the first end of the upper portion. The second conduit extends downwardly into a central column or inner annular space or cavity defined within the at least one inner structure. In one embodiment, the second conduit has a second length such that a second end of the second conduit is at a second level of the housing. Additionally, the outer conduit may have a first length such that a first end of the outer conduit is at a first level of the housing that is different than the second level.

In one embodiment, the second conduit includes a sensor to measure a condition of fluid in the central column. The sensor is operable to measure at least one of pH, dissolved oxygen (DO), dissolved CO₂, glucose, lactate, glutamine, ammonia, and temperature. The sensor may be positioned between an upper end and a lower end of the inner structure. Optionally, the second conduit is transparent or translucent to transmit light from the sensor to a reader positioned within the second conduit. Additionally, or alternatively, the second conduit has a lumen and an orifice to withdraw fluid from the central column.

Optionally, the settler device includes a plurality of second conduits extending into central column. In one embodiment, each of the plurality of second conduits has a different length such that each of the second conduits can sample a condition or withdraw fluid a different level of the central column.

In one embodiment the settling device further comprises a first distributor element that is positioned within the settling device. The first distributor element is operable to introduce fluid into or withdraw fluid from the settling device. The first distributor element includes a plurality of apertures sized for a gas to flow therethrough. Optionally, one or more of air, O₂, CO₂ and N₂ may be introduced into the settling device through the first distributor element.

Additionally, or alternatively, the settling device may include a second distributor element positioned within the settling device. In one embodiment, the second distributor element is separate from the first distributor element. In another embodiment, the second distributor element may be positioned at a separate height within the settling device than the height of the first distributor element. The second distributor element is operable to introduce fluid into or withdraw fluid from the settling device. The second distributor element includes a plurality of apertures sized for a gas to flow therethrough. Optionally, one or more of air, O₂, CO₂ and N₂ may be introduced into the settling device through the second distributor element.

Another aspect of the present disclosure in a method of settling particles in a suspension, comprising: (1) introducing a liquid suspension of particles into a settling device which includes: (i) a lower section with a port; (ii) a housing with a lower end contacting and extending upwardly from the lower portion, an upper end, and an interior wall; (iii) inclined sliders provided within the housing, each inclined slider including (a) a body with an arcuate surface having a groove and sides spaced from a first adjacent inclined slider, and at least one of (b) an edge coupled to the interior surface of the housing and/or (c) an edge coupled to the exterior surface of at least one inner structure installed within the housing, and (d) a second arcuate surface spaced from a second adjacent inclined slider; (iv) an upper portion connected to the upper end of the housing; (v) a first conduit extending from the upper portion; (vi) an second conduit extending from the upper portion and downwardly in an annular space between the interior wall of the housing and an exterior surface of the at least one inner structure; and (vii) a sensor associated with the second conduit; (2) measuring one or more of pH, dissolved oxygen (DO), dissolved CO₂, glucose, lactate, glutamine, ammonia and temperature in the annular space with the sensor associated with the outer conduit; (3) collecting a clarified liquid through the orifice of the first conduit; and (4) collecting a concentrated liquid suspension from the port of the lower portion.

In one embodiment, the liquid suspension comprises at least one of a recombinant cell suspension, an alcoholic fermentation, a suspension of solid catalyst particles, a municipal wastewater, industrial wastewater, mammalian cells, bacterial cells, yeast cells, plant cells, algae cells, plant cells, mammalian cells, murine hybridoma cells, stem cells, CAR-T cells, red blood precursor and mature cells, cardiomyocytes, yeast in beer, and eukaryotic cells.

Additionally, or alternatively, the liquid suspension may comprise one or more of recombinant microbial cells selected from at least one of Pichia pastoris, Saccharomyces cerevisiae, Kluyveromyces lactis, Aspergillus niger, Escherichia coli, and Bacillus subtilis.

In one embodiment, the liquid suspension may comprise one or more of microcarrier beads, affinity ligands, and surface activated microspherical beads. In some embodiments, the liquid suspension may be an emulsion of organic and aqueous phases, which are easily separated into the two separate phases inside the settlers described through the present disclosure. The aqueous phase is typically cell culture or fermentation broth containing live and productive cells, while the organic phase is added to the bioreactor to extract any toxic metabolic products, such as terpenes produced by metabolically engineered yeast cells. As the toxic metabolite product is continuously extracted into the organic phase, its concentration in aqueous phase is decreased, thereby allowing the live yeast cells to grow and produce more metabolite product in the perfusion bioreactor.

Additionally, or alternatively, the clarified liquid collected comprises at least one of biological molecules, organic or inorganic compounds, chemical reactants, chemical reaction products, hydrocarbons (e.g., terpenes, isoprenoids, polyprenoids), polypeptides, proteins (e.g., brazzein, colony stimulating factors), alcohols, fatty acids, hormones (e.g., insulin, growth factors), carbohydrates, glycoproteins (e.g., erythropoietin, monoclonal antibodies), beer, and biodiesel.

The method may further comprise controlling at least one of pH, dissolved oxygen, dissolved CO₂, glucose, lactate, glutamine, and ammonia within the settler device. In one embodiment, the controlling comprises at least one of: (i) manipulating the flow rates of a gas into the settler device; and (ii) manipulating the flow rates of different liquid media components into the settler device. Optionally, the gas is at least one of air, O₂, CO₂ and N₂. The gas may be introduced through a first distributor element. In one embodiment, the liquid media components are pumped in through a second distributor element. In one embodiment, introducing a liquid suspension of particles into the settling device comprises pumping the liquid suspension through a second distributor element positioned within the settling device.

In some embodiments, the method further comprises measuring one or more of pH, dissolved oxygen (DO), dissolved CO₂, glucose, lactate, glutamine, ammonia, and temperature in the housing with a sensor associated with a second conduit extending from the upper portion.

Additionally, or alternatively, the method may include measuring one or more of pH, dissolved oxygen (DO), dissolved CO₂, glucose, lactate, glutamine, ammonia, and temperature in the housing with a sensor positioned in the housing. In one embodiment, the sensor is associated with the first port or conduit. Alternatively, the sensor is associated with a third conduit extending from the lid or top closure or upper portion into the housing.

In one embodiment, the lid or top closure or upper portion or upper portion has a shape that is conical. The conical upper portion has a first end that has a first diameter and a second end with a second diameter that is larger than the first diameter. In one embodiment, the first end is oriented toward the lower conical portion.

Another aspect of the present disclosure is a particle settling device that may include, but is not limited to, a housing including one or more of: (1) a first series of inclined sliders; (2) a second series of inclined sliders; (3) a first inner structure, where the first series of inclined sliders is located between a housing surface and a first surface of the first inner structure; (4) a second inner structure, where the second series of inclined sliders is located between a second surface of the first inner structure and a surface of the second inner structure; at least one input port or conduit for introducing a liquid into the housing; and (5) at least one outlet port or conduit for removing material from the housing. In one embodiment, a first outlet port is associated with the first series of inclined settlers and a second outlet port is associated with a second series of inclined settlers. Optionally, the liquid introduced into the housing may be a liquid suspension including particles. The particles may be of a plurality of sizes.

In one embodiment, a first outlet port may be for harvesting a clarified liquid. The clarified liquid may include a first subset of particles. The first subset of particles may comprise cell debris, dead cells, and the like. Optionally, the first outlet port may be formed in a closure coupled to the housing. The first outlet port being in fluidic communication with the outside and the inside of the housing.

Optionally, in another embodiment, a second outlet port may be for harvesting a concentrated liquid. The concentrated liquid may include a second subset of particles, such as live cells. Typically, particles of the second subset of particles are generally larger than particles of the first subset of particles. Each particle of the second subset of particles generally has a greater mass than the particles of the first subset of particles. The second outlet port is in fluidic communication with the outside and the inside of the housing.

Optionally, an angle of inclination for a surface of the inclined sliders in the first series or the second series may vary between about 15 degrees to about 75 degrees from vertical. In one embodiment, the surface of the inclined sliders is convex or concave such that a cross-section of the inclined slider defines an arcuate line. In another embodiment, the angle of inclination of the surface of the inclined sliders in the first series or the second series may be constant at any angle between 15 and 75 degrees from vertical. In one embodiment, the angle of inclination of the surface of the inclined sliders in the first series or the second series is about 45 degrees.

In one embodiment, the inclined sliders in the first series or the second series have a substantially uniform spacing. In one embodiment, the inclined sliders in the first series have a different spacing compared to the inclined sliders in the second series.

At least one port is configured as an inlet port in fluidic communication with the outside and the inside of the housing. The at least one port may be associated with at least one of the first series of inclined sliders, the second series of inclined sliders, a first cavity defined between sidewalls of the housing and the first inner structure, or a second cavity defined between the first inner structure and the second inner structure. In one embodiment, a first inlet of the at least one port is associated with at least one of the first cavity or the second cavity. In yet another embodiment, the second inlet is associated with at least one of the first cavity or the second cavity. In another embodiment, the at least one port is configured to be interconnected to a disposable bioreactor bag. The disposable bioreactor bag may comprise a plastic material.

Another aspect of the present disclosure is a settling device operable for use in the production of cell therapy products, secreted biological proteins, polypeptides or hormones, comprising: (1) an top closure or upper portion with a central port and at least one peripheral port; (2) a housing or outer structure; (3) at least one inner structure; (4) a bottom closure or lower portion with a middle port and at least one outer port; and (4) a series of sliders, the series of sliders generally centered around and set at an incline to a longitudinal axis of the settling device defined by the housing.

In one embodiment, the lid or top closure or upper portion and/or the bottom closure or lower portion is configured to couple to the housing via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the lid or top closure or upper portion and/or the bottom closure or lower portion to the housing may be used with the settler device. Optionally, a washer or gasket may be positioned between the housing and the top closure and/or the bottom closure during assembly of the settler device.

In one embodiment, the settling device includes an impeller with one or more sets of blades to agitate input fluids. In another embodiment, the housing and/or the inner structure may include alignment fins or brackets configured to guide the sliders when installed into the housing and/or prevent the sliders from moving when internal fluid is agitated with an impeller.

Another aspect of the present disclosure is a settling device which includes, but is not limited to: (1) a housing; (2) a top closure or upper portion interconnectable to the housing including at least one port or conduit; an inner structure or separation plate configured to separate a cavity defined within the housing into at least first and second cavity portions; (3) a series of inclined sliders located within the first cavity portion; and (4) an impeller located within the second cavity portion. Optionally, the settler device may include (5) a bottom closure or lower portion that is fixed to the housing or interconnectable to the housing, and including at least one port or conduit.

In these devices, a surface of an inclined slider is at an angle of between approximately 15 degrees to about 75 degrees relative to a longitudinal axis defined by the housing. Optionally, the inclined slider is concave or convex such that a cross-section of the include slider defines an arcuate line.

In any of the settler devices of this disclosure, the housing and/or the inclined sliders and/or any other components of the settler devices may be composed of a metal or a plastic. The plastic may be one or more of polypropylene, polyethylene, polycarbonate, polystyrene, and the like. Optionally, the plastic components may be disposable. In one embodiment, the settling device is formed entirely of plastic. In another embodiment, at least one inclined slider is composed at least partially of stainless steel. The metal surfaces (especially stainless steel) may be electropolished to provide a smooth surface. Similarly, in any of the settler devices of this disclosure, the housing and/or the inclined sliders and/or any other components of the device may be completely or partially coated with one or more of a non-sticky plastic, such as Teflon or silicone.

In any of the settler devices of this disclosure, the housing may further include a fluid jacket associated with one or more of a housing, a first series of inclined sliders, a second series of inclined sliders, and/or an inner structure. In one embodiment, the fluid jacket is associated with at least the housing. The fluid jacket may include at least one port to receive a fluid of a predetermined temperature. Optionally, the fluid jacket may include a second port to extract fluid from the fluid jacket. Water or other fluids may be directed into the fluid jacket to maintain the housing and all of its contents within a desired temperature range. Ports may be formed in the outer wall of the housing to reach the jacket. The ports may function as inlet or outlet ports for the circulation of cooling or heating fluids through the jacket.

In any of the settler devices of this disclosure, one or more sensors may be positioned to monitor physical conditions within the interior of the settler device. Additionally, or alternatively, at least one sensor may be positioned to monitor conditions within a tubing line interconnected to the settler devices of this disclosure. The tubing line may be a return line interconnected to a bottom outlet port of the settler device.

These sensors may be selected to determine one or more of pH, dissolved oxygen (DO), glucose, temperature, CO₂ (including dissolved CO₂, known as partial CO₂), glucose, lactate, glutamine, and ammonia within a housing of the settler device or a tubing line connected to the settler device. The sensors may include one or more probes in contact with a solution within the housing or the tubing line. The probe may be affixed to an interior surface of the settler device or the tubing line. In preferred embodiments, at least one sensor and/or probe is positioned within the bottom closure or lower portion of the settler device, and may be spaced from one or more of a side port or a bottom port.

These probe(s) may transmit data without contact to a reader. In this manner, the probe may measure a condition within the settler device and/or the line and transmit data to the reader outside the settler device. One or more of the probes may be a fluorescent probe. One or more of pH, DO, glucose, lactate, glutamine, ammonia, temperature, and pCO₂ may be measured by the probe within the settler device. The probe may be affixed to a portion of the housing. The portion of the housing may be operable to transmit light produced by the fluorescent probe. As described herein, a portion of the housing may be transparent or translucent. The reader (or meter) receives light from the fluorescent probe. The reader may also include an optical fiber that collects light transmitted by the fluorescent probe.

Any suitable sensor or probe known to those of skill in the art may be used with the settler devices of the present disclosure. Suitable probes and readers are available from a variety of vendors, including Advanced Biosensors, Scientific Bioprocessing, Inc., and PreSens Precision Sensing GmbH. In another configuration, the probe within the settler device can transmit data to the reader outside the settler device by a network connection. For example, the probe can communicate with the reader by WiFi, Bluetooth, or any other wired or wireless communication modality.

In operation of a settler device of this disclosure, data from these sensor(s) may be used to adjust a temperature of fluid within the fluid jacket. In another embodiment, the data from the sensor may be used to adjust or control one or more of pH, temperature, dissolved oxygen concentration, dissolved carbon dioxide, and nutrient concentrations within the particle settling device. For example, flow rates of fluids into or out of the settler device may be altered to adjust or control one or more of pH, temperature, dissolved oxygen concentration, dissolved carbon dioxide, and nutrient concentrations within the settling device. Additionally, or alternatively, the flow rates of at least one of air, O₂, CO₂, and/or N₂ into the settling device may be adjusted to control conditions within the settling device.

Another aspect is a method of settling particles in a suspension, comprising: (1) introducing a liquid suspension of particles into a settler device which includes: (i) an upper portion with a central port and at least one peripheral port; (ii) a housing and at least one inner structure installed within the housing; (iii) a lower portion with a middle port and at least one outer port; (iv) inclined sliders located within the housing, each inclined slider including (a) a body with an arcuate surface having a groove and sides spaced from a first adjacent inclined slider, and at least one of (b) an edge coupled to the interior surface of the housing and/or (c) an edge coupled to the exterior surface of at least one inner structure installed within the housing, and (d) a second arcuate surface spaced from a second adjacent inclined slider; and (e) an impeller for agitating the liquid suspension of particles; (2) collecting a clarified liquid from the central port of the upper portion; and (3) collecting a concentrated liquid suspension from the middle port of the lower portion.

In one embodiment, the liquid suspension comprises at least one of a recombinant cell suspension, an alcoholic fermentation, a suspension of solid catalyst particles, a municipal wastewater, industrial wastewater, mammalian cells, bacterial cells, yeast cells, plant cells, algae cells, plant cells, mammalian cells, murine hybridoma cells, stem cells, CAR-T cells, red blood precursor and mature cells, cardiomyocytes, yeast in beer, and eukaryotic cells.

In another embodiment, the liquid suspension comprises at least one of: (a) recombinant microbial cells selected from at least one of Pichia pastoris, Saccharomyces cerevisiae, Kluyveromyces lactis, Aspergillus niger, Escherichia coli, and Bacillus subtilis; and (b) one or more of microcarrier beads, affinity ligands, and surface activated microspherical beads. Optionally, introducing a liquid suspension comprises directing the liquid suspension through the at least one outer port of the lower portion at a first rate. In one embodiment, the concentrated liquid suspension is collected from a middle port at a second rate that is less than the first rate such that the clarified liquid flows out of a central port of the upper portion.

In one embodiment, the clarified liquid collected comprises at least one of biological molecules, organic or inorganic compounds, chemical reactants, chemical reaction products, hydrocarbons (e.g., terpenes, isoprenoids, polyprenoids), polypeptides, proteins (e.g., brazzein, colony stimulating factors), alcohols, fatty acids, hormones (e.g., insulin, growth factors), carbohydrates, glycoproteins (e.g., erythropoietin, monoclonal antibodies), beer, and biodiesel.

The method optionally further comprises withdrawing liquid from the settler device. In one embodiment, the method includes measuring at least one of pH, dissolved oxygen, dissolved CO₂, glucose, lactate, glutamine, ammonia, and temperature within the settler device by a sensor.

In another embodiment, introducing a liquid suspension of particles into the settler device comprises pumping the liquid suspension through at least one outer port of the lower portion and through a plurality of holes formed in a body of a distributor element. In one embodiment, the body of the distributor element include (i) a lower surface with a lower protrusion in contact with an interior surface of the lower portion of the settling device; and (ii) an upper surface with an upper protrusion in contact with an inclined slider.

One aspect of the present disclosure is a settler device for concentrating fluids and harvesting cells including: mesenchymal stem cells (MSCs), induced pluripotent stem cells (iPSCs), chimeric antibody receptor-T lymphocytes (CAR-T cells) and other stem cell or their products, such as organoids or exosomes, and cultured meat or fish cells.

The settler device is configured to: (i) reduce shear stress to cells within the settler device, decreasing damage and death of cells; (ii) selectively remove any previously generated dead cells and/or cell debris; and (iii) separate single live cells from larger microcarrier beads or cell aggregates (e.g., organoids) or subcellular products (e.g., extracellular vesicles or exosomes). By reducing shear stress and damage to cells, the settler devices of the present disclosure provide a higher percentage of viable cell therapy products, with higher therapeutic value, for the treatment of various cancers and other diseases compared to other settler devices.

The settler device can gently separate desired cells or particles from the spent culture media, from the microcarrier beads, and from dead cells and cell debris.

The settler device can recover about 95% of secreted antibody in the clarified supernatant. In one embodiment, the settler device can reduce the turbidity of the supernatant to below 200 NTUs from the starting cell culture broth turbidities of around 2,000 NTU, as required for successful downstream depth filtration as a secondary or final clarification step.

Another aspect of the present disclosure is a settler device which includes single use, disposable sensors for glucose, temperature, pH, and dissolved oxygen. The glucose, temperature, pH, dissolved oxygen, dissolved CO₂, lactate, glutamine, and ammonia within the settler device are controlled by manipulating input nutrient media and gas mixture sparged into the settler device. For example, by manipulating the flow rates of at least one of air, O₂, CO₂ and N₂ introduced into the settling device through a distributor or by manipulating the flow rates of different liquid media components pumped in through the distributor.

Another aspect of the present disclosure is to provide a bioreactor that has a housing with an internal settler assembly. The housing may be a single-piece construction or may include multiple sections that are separable. The internal settler assembly includes inclined arcuate sliders or settler plates coupled to an inner cylinder. The internal settler assembly is separable into multiple sections that are insertable into the housing. Each of the multiple sections may include a portion of the inner cylinder and one or more sliders. For example, the divider line between the sections may also intersect the one or more sliders and separate the one or more sliders into sections. By way of another example, the divider line between the section may be positioned within a gap between sliders, such that the sliders are not intersected.

Another aspect of the present disclosure includes a bioreactor that has a housing with an internal settler assembly. The housing may be a single-piece construction or may include multiple sections that are separable. The internal settler assembly includes ring sliders or settler plates surrounding an inner cylinder. The ring sliders includes an upper surface that is sloped relative to the inner cylinder. For example, the upper surface may be inclined or sloped downward in a direction toward the inner cylinder, such that settling particles may be directed toward the inner cylinder and the ring may be considered concave. By way of another example, the upper surface may be inclined or sloped upward in a direction away from the inner cylinder, such that settling particles may be directed away from the inner cylinder and the ring may be considered convex.

Another aspect of the present disclosure includes a bioreactor that has a housing with an internal settler assembly. The housing may be a single-piece construction or may include multiple sections that are separable. The internal settler assembly includes cone sliders or settler plates. The cone sliders may be upward-facing or downward-facing relative to a top opening of the housing. The housing includes a top lid or closure.

Another aspect of the present disclosure includes a bioreactor that has a housing with internal settler assembly. The internal settler assembly includes substantially planar sliders or settler plates. The substantially planar sliders may be inclined relative to a top surface of the housing. In some embodiments, the housing comprises an impeller with a circular or substantially circular cross-section. The impeller is positioned within the housing below the sliders or settler platers, within a lower section of the housing with an actuate bottom surface that corresponds to the cross-section of the impeller. The impeller may cause settling particles such as microcarrier particles or semi-solid single cells or cellular aggregates or organoids to recirculate, or may agitate the fluid and cause the settling particles to unseat in the bottom of the arcuate second section of the housing to be delivered to an outlet port (e.g., where the outlet port is positioned within the bottom section between the bottom edge of the sliders or settler plates and the impeller).

In certain embodiments, a settling device operable for use in the production of cell therapy products, secreted biological proteins, polypeptides or hormones, vaccines, viral vectors, or gene therapy products is disclosed. The settling device includes a lower section. The settling device includes a housing with a lower end contacting and extending upwardly from the lower section, an upper end, and an outer cylindrical wall. The settling device includes an inner structure positioned within the housing, where the inner structure includes an inner cylindrical wall, and where the inner structure and the housing are concentrically aligned along a longitudinal axis defined by the housing. The settling device includes inclined sliders provided within a cavity defined by an interior surface of the outer cylindrical wall and an exterior surface of the inner cylindrical wall. The settling device includes an upper section coupled to the upper end of the housing.

In certain embodiments, a bioreactor operable for use in the production of cell therapy products, secreted biological proteins, polypeptides or hormones, vaccines, viral vectors, or gene therapy products is disclosed. The bioreactor includes a lower section. The bioreactor includes a housing with a lower end contacting and extending upwardly from the lower section, an upper end, and an outer cylindrical wall. The bioreactor includes an inner structure positioned within the housing. The bioreactor includes inclined sliders provided within a cavity defined at least by an interior surface of the outer cylindrical wall. The bioreactor includes an impeller positioned in the cavity, the impeller including one or more sets of blades. The bioreactor includes an upper portion coupled to the upper end of the housing.

In certain embodiments, a method of settling particles in a suspension is disclosed. The method may include, but is not limited to, introducing a liquid suspension of particles into a settling device. The settling device includes a lower section with a first port. The settling device includes a housing with a lower end contacting and extending upwardly from the lower section, an upper end, and an outer cylindrical wall. The settling device includes an inner structure positioned within the housing, where the inner structure includes an inner cylindrical wall, and where the inner structure and the housing are concentrically aligned along a longitudinal axis defined by the housing. The settling device includes a plurality of inclined sliders provided within a cavity defined by an interior surface of the outer cylindrical wall and an exterior surface of the inner cylindrical wall. The settling device includes an upper section coupled to the upper end of the housing, the upper portion including at least a second port. The settling device includes a sensor. The method may include, but is not limited to, measuring one or more of pH, dissolved oxygen (DO), dissolved CO₂, glucose, lactate, glutamine, ammonia, and temperature in the cavity with the sensor. The method may include, but is not limited to, collecting a clarified liquid from the at least a second port. The method may include, but is not limited to, collecting a concentrated liquid suspension from the first port.

In certain embodiments, a settling device is provided inside a bioreactor for use in the production of cell therapy products, secreted biological proteins, polypeptides or hormones, vaccines, viral vectors, or gene therapy products that comprises a housing with a lower end, an upper end, and an outer cylindrical wall. The settling device comprises an inner structure positioned within the housing, the inner structure including an inner cylindrical wall, wherein the inner structure and the housing are concentrically aligned along a longitudinal axis defined by the housing. The settling device comprises a plurality of sliders provided within a cavity defined by an interior surface of the outer cylindrical wall and an exterior surface of the inner cylindrical wall. The settling device comprises an upper section coupled to the upper end of the housing.

In certain embodiments, a perfusion bioreactor operable for use in the production of cell therapy products, secreted biological proteins, polypeptides or hormones, vaccines, viral vectors, or gene therapy products comprises a housing with a lower end, an upper end, and an outer wall. The perfusion bioreactor comprises a plurality of sliders provided within a cavity defined at least by an interior surface of the outer wall. For purposes of the present disclosure, a perfusion bioreactor may be understood as a device that circulates a fluid throughout the housing while particles settle from the fluid on the plurality of sliders.

In certain embodiments, a method for settling particles or cells in a suspension may include, but is not limited to, introducing a liquid suspension of particles or cells into a settling device. The settling device may comprise a housing with a lower end, an upper end, and an outer wall. The settling device may comprise an inner structure positioned within the housing, the inner structure including an inner cylindrical wall, wherein the inner structure and the housing are concentrically aligned along a longitudinal axis defined by the housing. The settling device may comprise a plurality of sliders provided within a cavity defined by an interior surface of the outer wall and an exterior surface of the inner cylindrical wall. The settling device may comprise a sensor. The method may include, but is not limited to, measuring one or more of pH, dissolved oxygen (DO), dissolved CO₂, glucose, lactate, glutamine, ammonia, and temperature in the cavity with the sensor. The method may include, but is not limited to, collecting a clarified liquid from the at least a second port. The method may include, but is not limited to, collecting a concentrated liquid suspension from the first port.

The preceding is a simplified summary of the disclosure intended to provide an understanding of some aspects of the settler devices of this disclosure. This Summary is neither an extensive nor exhaustive overview of the invention and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below. As will be appreciated, other embodiments are possible using, alone or in combination, one or more of the features set forth above or described herein. For example, it is contemplated that various features and devices shown and/or described with respect to one embodiment may be combined with or substituted for features or devices of other embodiments regardless of whether or not such a combination or substitution is specifically shown or described herein. Additional aspects of the present invention will become more readily apparent from the Detailed Description, particularly when taken together with the drawings.

### BRIEF DESCRIPTION OF FIGURES

FIG. 1A is a top view of a single clockwise curved spiral slide settler of a particle settling device according to embodiments of the present disclosure;

FIG. 1B is a side view of the single clockwise curved spiral slide settler of FIG. 1A;

FIG. 1C is a top view of a single counterclockwise curved spiral slide settler of a particle settling device according to embodiments of the present disclosure;

FIG. 1D is a side view of the single counterclockwise curved spiral slide settler of FIG. 1C;

FIG. 2 is a portion of a particle settling device with a single clockwise curved spiral slide settler according to embodiments of the present disclosure;

FIG. 3 is a portion of a particle settling device with three clockwise curved spiral slide settlers according to embodiments of the present disclosure;

FIG. 4 is a portion of a particle settling device with clockwise curved spiral slide settlers according to embodiments of the present disclosure;

FIG. 5 is a cross-section view of a side elevation view of the particle settling device of FIG. 4;

FIG. 6 is a top plan view of the particle settling device of FIG. 4;

FIG. 7 is a top plan view of a particle settling device according to embodiments of the present disclosure;

FIG. 8 is a side perspective view of a particle settling device according to embodiments of the present disclosure;

FIG. 9 is a side perspective view of a portion of a particle settling device of FIG. 8 with multiple top ports;

FIG. 10 is a side perspective view of a particle settling device with a single clockwise curved multiple-ridged spiral slide settler according to embodiments of the present disclosure;

FIG. 11 is a top plan view of the particle settling device of FIG. 10;

FIG. 12 is a side perspective view of a particle settling device with multiple top ports according to embodiments of the present disclosure;

FIG. 13 is a cross-section of a side perspective view of the particle settling device of FIG. 12;

FIG. 14 is a cross-section of a side perspective view of the particle settling device of FIG. 12;

FIG. 15 is a front elevation view of a bioreactor with multiple sliders according to embodiments of the present disclosure;

FIG. 16 is a side perspective view of the bioreactor of FIG. 15;

FIG. 17 is a back right perspective view of the bioreactor of FIG. 15;

FIG. 18 is a back left perspective view of the bioreactor of FIG. 15;

FIG. 19 is the back left perspective view of the bioreactor of FIG. 15 with a top removed;

FIG. 20 is a top plan view of a portion of the bioreactor of FIG. 15;

FIG. 21 is a perspective front view of clockwise curved spiral slide settlers of the bioreactor of FIG. 15;

FIG. 22 is a perspective rear lower view of clockwise curved spiral slide settlers of FIG. 15;

FIG. 23 is a perspective view of a portion of a bioreactor with a single counterclockwise curved spiral slide settler according to embodiments of the present disclosure;

FIG. 24 is a perspective view of a portion of the bioreactor of FIG. 23 with multiple counterclockwise curved spiral slide settlers;

FIG. 25 is a perspective view of the bioreactor of FIG. 23;

FIG. 26 is a side elevation view of the bioreactor of FIG. 25;

FIG. 27 is a cross section of front elevation view of the bioreactor of FIG. 25;

FIG. 28 is a top plan view of the bioreactor of FIG. 25;

FIG. 29 is a perspective view of a bioreactor with a single clockwise curved spiral slide settler according to embodiments of the present disclosure;

FIG. 30 is a perspective view of the bioreactor of FIG. 29 with multiple clockwise curved spiral slide settlers;

FIG. 31 is a cross section of a side perspective view of the bioreactor of FIG. 30;

FIG. 32 is a cross section of a side perspective view of a portion of a variation on the bioreactor of FIG. 30;

FIG. 33 is a cross section of a side elevation view of a portion of a bioreactor during isolated agitation according to embodiments of the present disclosure;

FIG. 34 is a cross section of a side elevation view of a portion of the bioreactor of FIG. 33 during incorporated agitation;

FIG. 35 is an example schematic of a perfusion bioreactor system including a settler device within a bioreactor according to embodiments of the present disclosure;

FIG. 36 is a side perspective view of a bioreactor with multiple clockwise spiral slide settlers about a separation cylinder according to embodiments of the present disclosure;

FIG. 37 is a top perspective view of the bioreactor of FIG. 36;

FIG. 38 is an exploded side perspective view of a bioreactor and the multiple clockwise spiral slide settlers about the separation cylinder of FIG. 36;

FIG. 39 is a top plan view of the bioreactor of FIG. 36 including section line B-B;

FIG. 40 is a side elevation view of the bioreactor of FIG. 36 viewed from the section line B-B of FIG. 39;

FIG. 41 is a top perspective view of the multiple clockwise spiral slide settlers about the separation cylinder of the bioreactor of FIG. 36;

FIG. 42 is a top perspective view of the multiple clockwise spiral slide settlers about the separation cylinder of FIG. 41 split into sections;

FIG. 43 is a top plan view of the bioreactor of FIG. 38 depicting the installation of the sections of the multiple clockwise spiral slide settlers about the separation cylinder;

FIG. 44 is a top plan view of the bioreactor of FIG. 38 depicting installed sections of the multiple clockwise spiral slide settlers about the separation cylinder;

FIG. 45 is a side perspective view of multiple ring slide settlers about a separation cylinder according to embodiments of the present disclosure;

FIG. 46 is a partial cut-away of the side perspective view of the multiple ring slide settlers about the separation cylinder of FIG. 45 installed within a bioreactor;

FIG. 47 is a cross-section of the multiple ring slide settlers about the separation cylinder of FIG. 45 installed within the bioreactor of FIG. 46;

FIG. 48 is a side perspective view of a variation of the separation cylinder of FIG. 45 with multiple ring slide settlers about the separation cylinder;

FIG. 49 is a partial cut-away of the side perspective view of the multiple ring slide settlers about the separation cylinder of FIG. 48 installed within a bioreactor;

FIG. 50 is a cross-section of the multiple ring slide settlers about the separation cylinder of FIG. 48 installed within the bioreactor of FIG. 49;

FIG. 51 is a side perspective view of a biosettler with multiple downward-pointed cone slide settlers according to embodiments of the present disclosure;

FIG. 52 is a partial cut-away of the side perspective view of the biosettler with the multiple downward-pointed cone slide settlers of FIG. 51;

FIG. 53 is a cross-section of the side perspective view of the biosettler with the multiple downward-pointed cone slide settlers of FIG. 51;

FIG. 54 is a perspective view of a bioreactor with slide settler plates according to embodiments of the present disclosure; and

FIG. 55 is a cross-section view of the bioreactor with the slide settler plates of FIG. 54.

### DETAILED DESCRIPTION

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

The term “a” or “an” entity, as used herein, refers to one or more of that entity. As such, the terms “a” (or “an”), “one or more” and “at least one” can be used interchangeably herein.

The transitional term “comprising” is synonymous with “including,” “containing,” or “characterized by,” is inclusive or open-ended and does not exclude additional, unrecited elements or method steps.

Unless otherwise indicated, all numbers expressing quantities, dimensions, conditions, ratios, ranges, and so forth used in the specification and claims are to be understood as being modified in all instances by the term “about” or “approximately”. Accordingly, unless otherwise indicated, all numbers expressing quantities, dimensions, conditions, ratios, ranges, and so forth used in the specification and claims may be increased or decreased by approximately 5% to achieve satisfactory results. Additionally, where the meaning of the terms “about” or “approximately” as used herein would not otherwise be apparent to one of ordinary skill in the art, the terms “about” and “approximately” should be interpreted as meaning within plus or minus 5% of the stated value.

It is noted that the systems and methods including between 1 and n number of a particular component should be understood has including 1, 2, ... and up to an n number of the particular component, where n may be any number, without departing from the scope of the present disclosure.

All ranges described herein may be reduced to any sub-range or portion of the range, or to any value within the range without deviating from the invention. For example, the range “5 to 55” includes, but is not limited to, the sub-ranges “5 to 20” as well as “17 to 54.”

The transitional phrase “consisting of” excludes any element, step, or ingredient not specified in the claim, but does not exclude additional components or steps that are unrelated to the disclosure such as impurities ordinarily associated therewith.

The transitional phrase “consisting essentially of” limits the scope of a claim to the specified materials or steps and those that do not materially affect the basic and novel characteristic(s) of the claimed invention.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

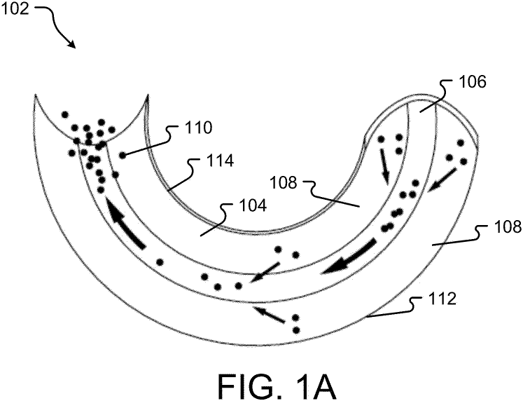

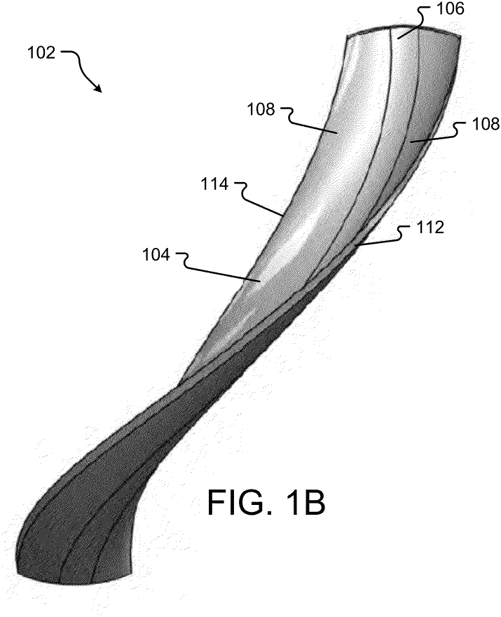

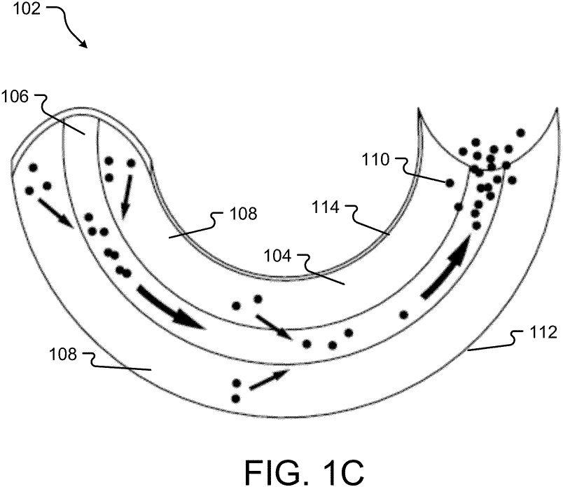

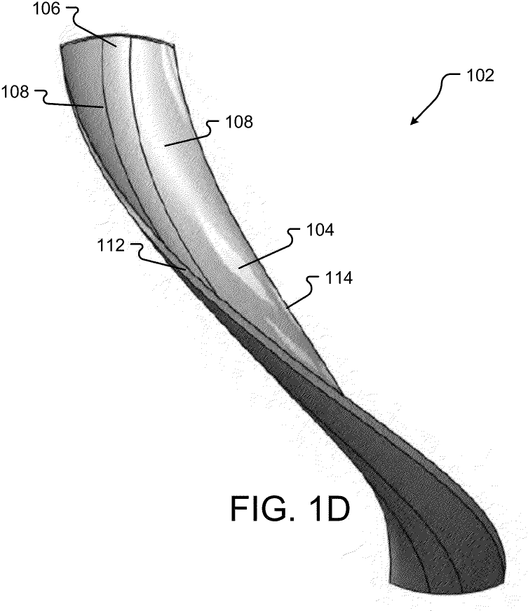

FIGS. 1A-14 in general illustrate a particle settler device 100 according to embodiments of the present disclosure. Embodiments directed to a bioreactor 600, a bioreactor 700, a bioreactor 800, a bioreactor 1000, a bioreactor 1100, a biosettler 1200, and/or a bioreactor 1300 may be applied to the settler device 100 unless otherwise noted. In particular, the settler device 100 is similar to other settler devices or bioreactors described herein and includes many of the same or similar features. In certain embodiments, one or more of the bioreactors 600, 700, 800, 1000, 1100, 1300 may be considered a “commercial bioreactor,” such as when one or more components of the bioreactor (e.g., a housing, lid or closure, or the like) is a commercially available product.

Referring now to FIGS. 1A-1D, an inclined spiral slide settler 102 (or slider 102) of the particle settler device 100 is illustrated. It is noted the slider 102 as described throughout the present disclosure may be incorporated or inserted into any suspension bioreactor.

The slider 102 includes a convex or concave surface 104 such that a cross-section of the slider 102 defines an arcuate line. The surface 104 includes a groove 106 and sides 108. For example, the groove 106 and the sides 108 may be an arcuate shape with a constant radius of curvature or multiple radii of curvature. By way of another example, the groove 106 may be set at an angle from the sides 108, with the groove 106 and/or the sides 108 being a curved surface or a planar or flat surface.

The particle settler device 100 is useful for settling particles or cells. The surface 104 directs particles 110 along the length of the slider 102 (e.g., from a higher level of the particle settler device 100 to a lower level of the particle settler device 100). For example, as represented by the arrows in FIGS. 1A and 1C, the sides 108 may direct the particles 110 inward toward the groove 106, and the groove 106 may direct the particles 110 along the length of the slider 102.

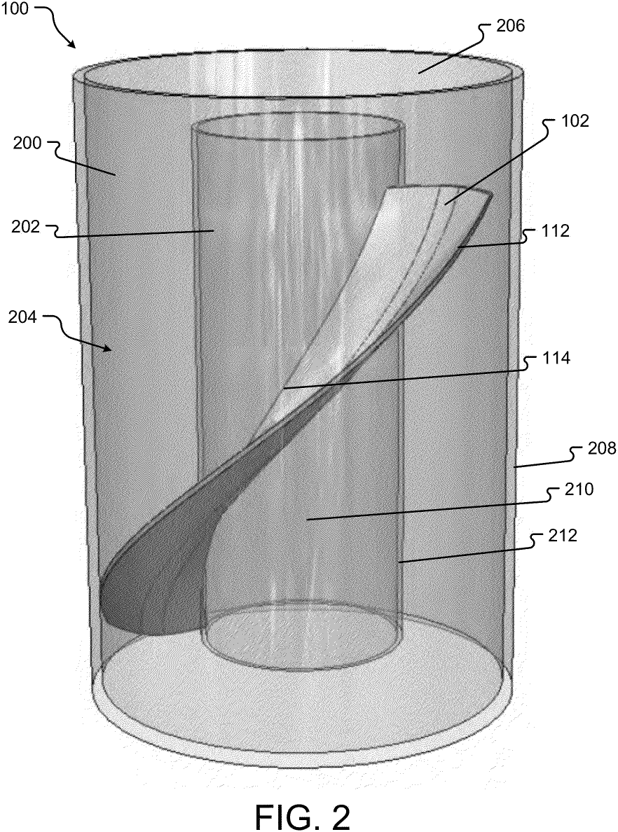

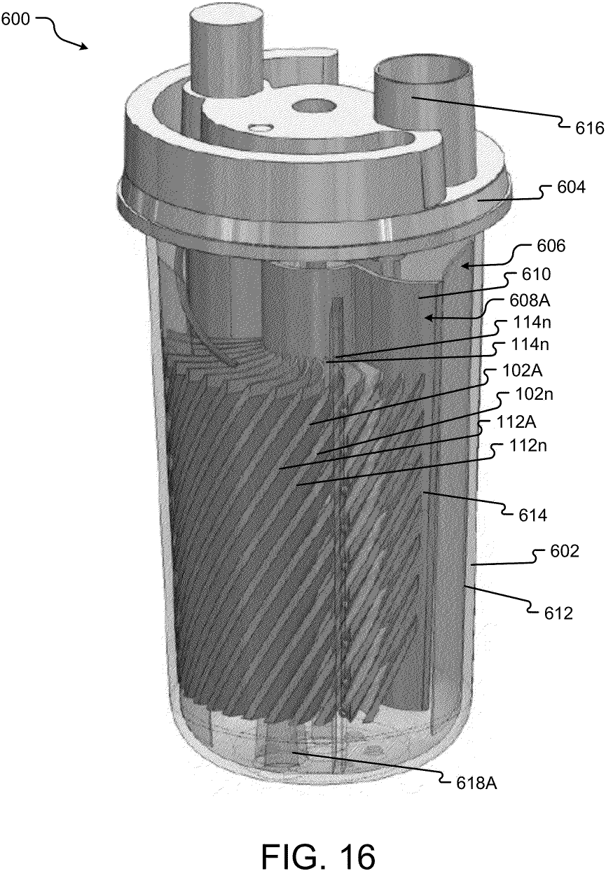

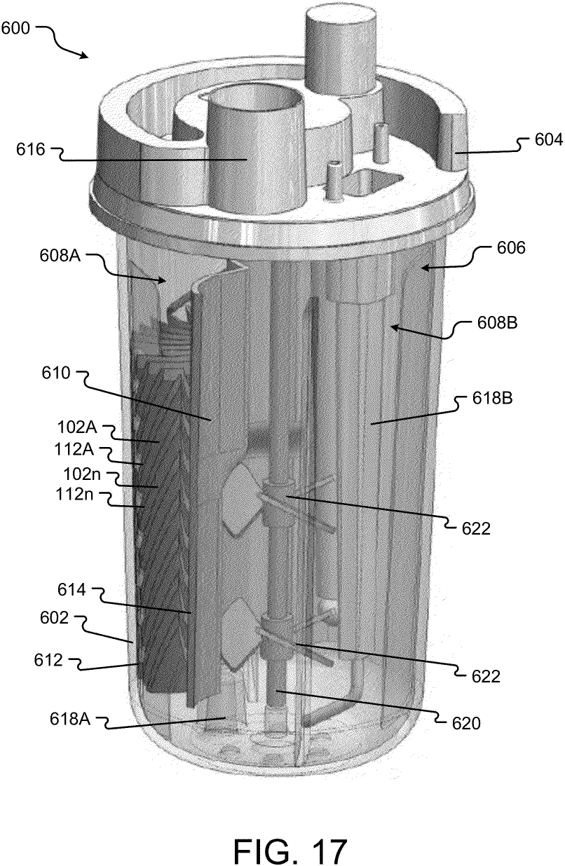

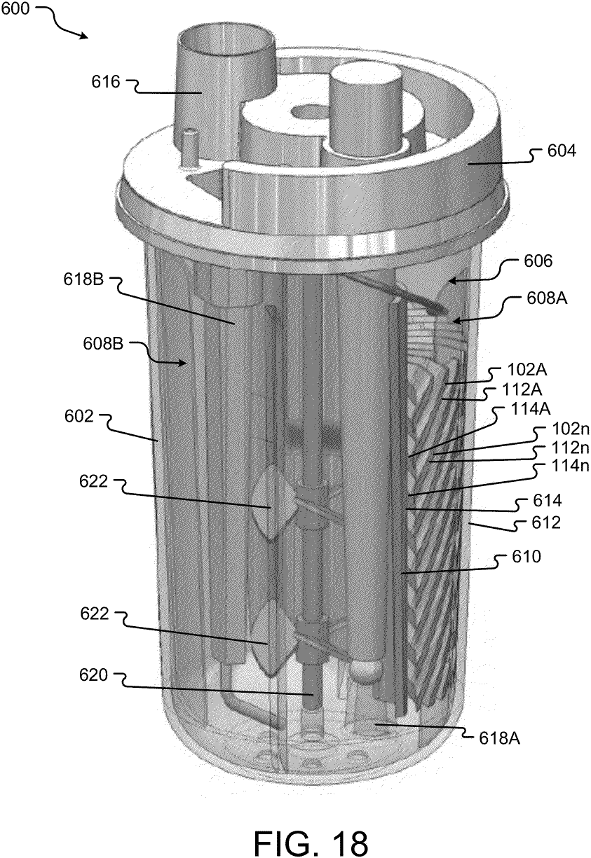

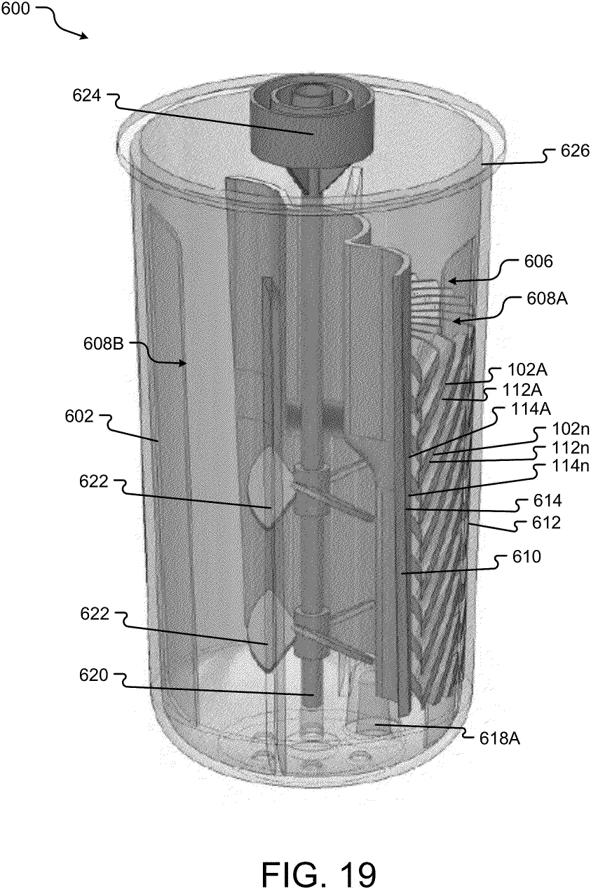

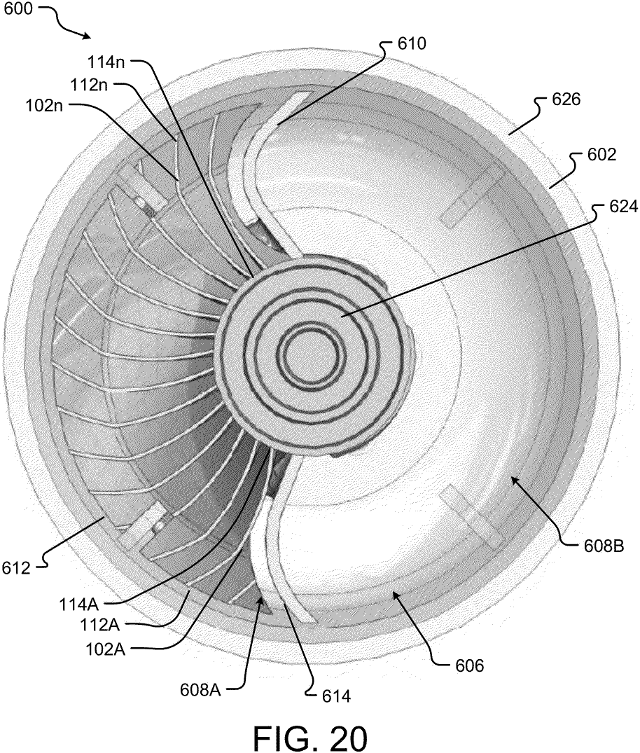

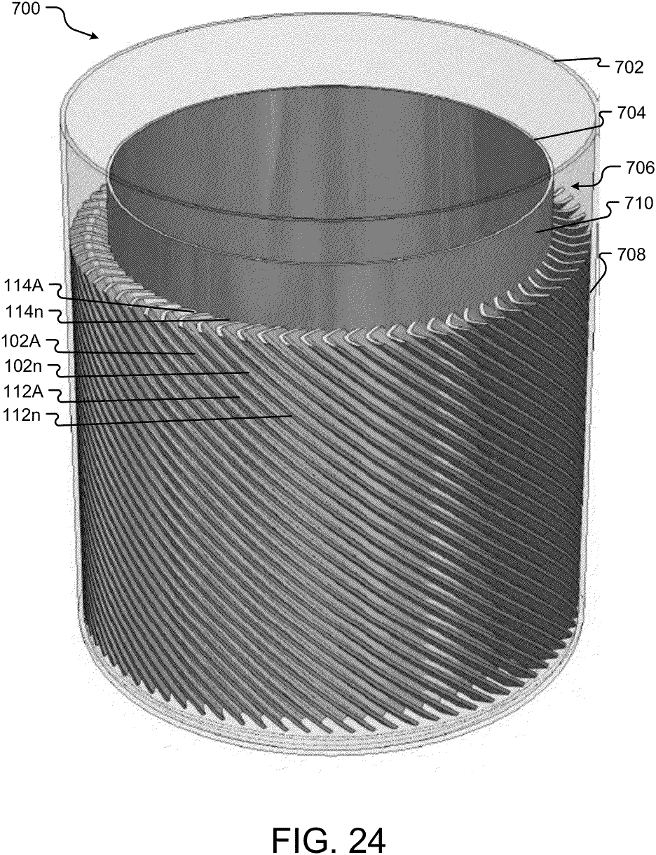

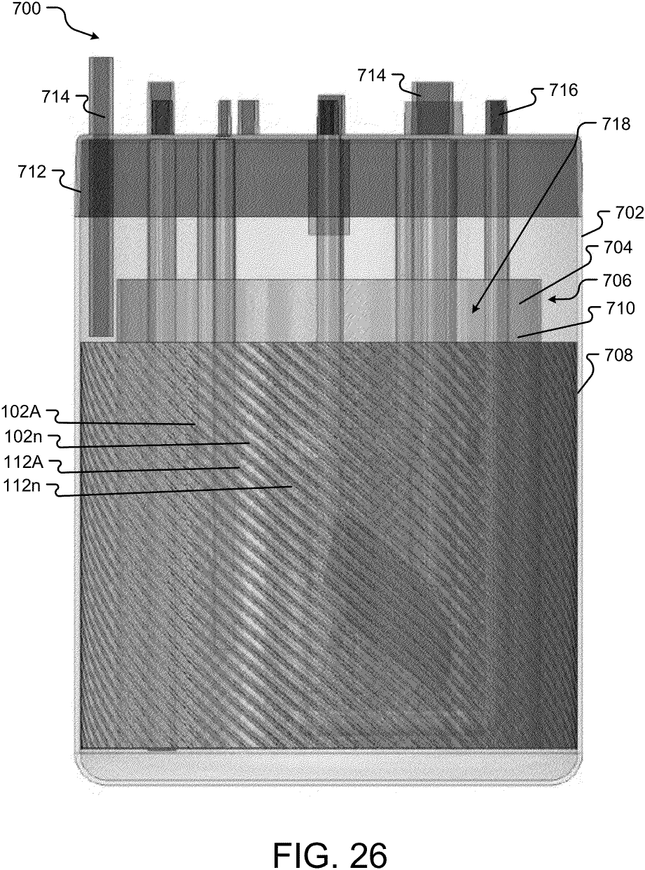

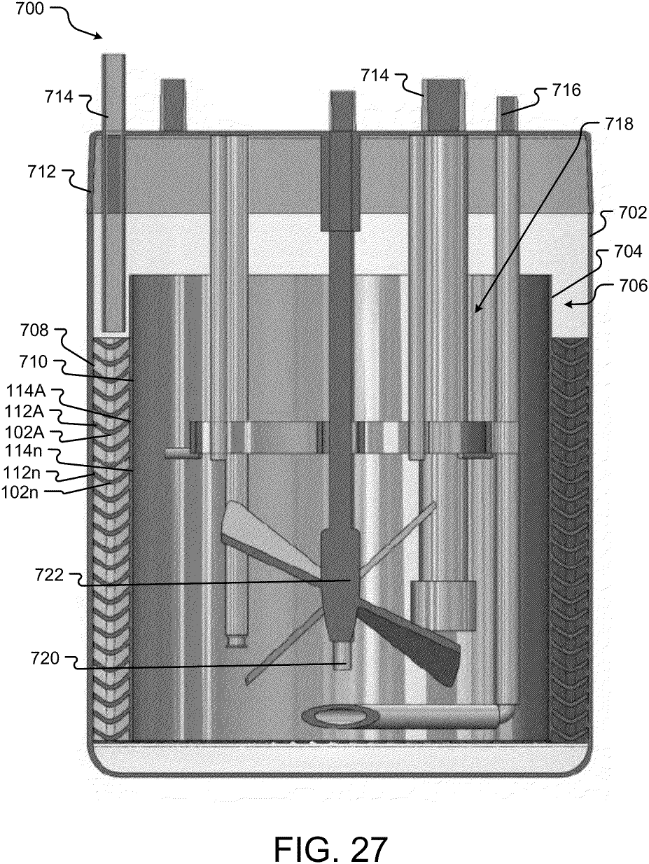

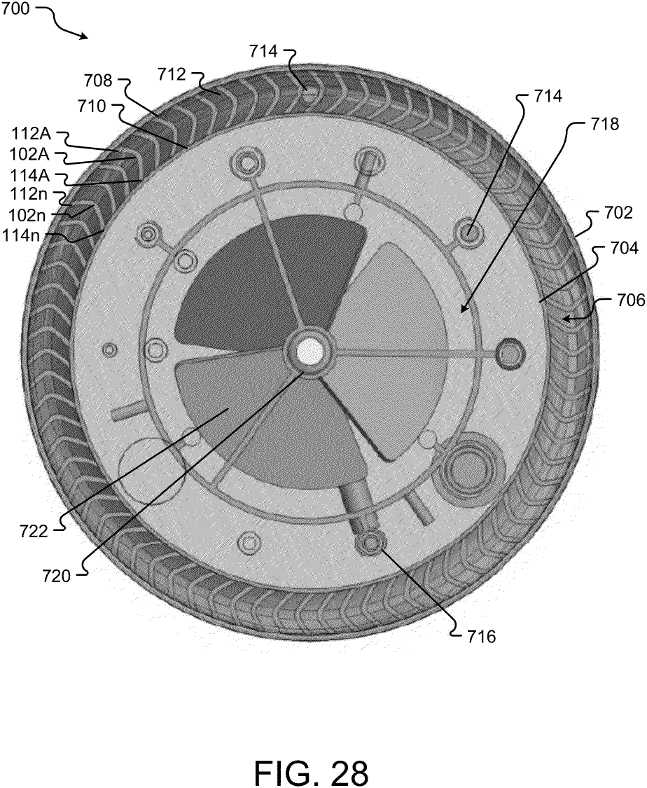

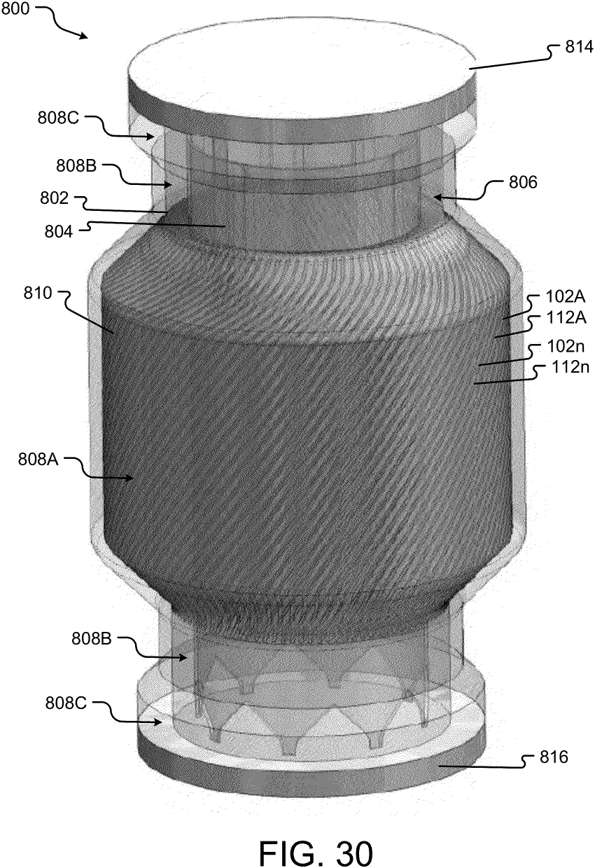

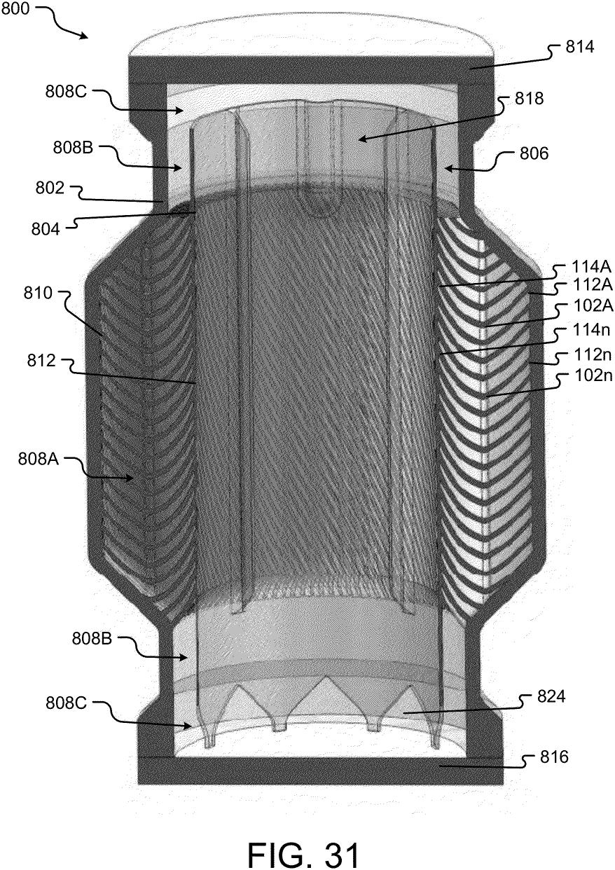

The slider 102 is oriented and arranged within the particular settler device 100 in a particular direction. For example, as illustrated in FIGS. 1A and 1B, the slider 102 may be oriented and arranged in a clockwise direction. By way of another example, as illustrated in FIGS. 1C and 1D, the slider 102 may be oriented and arranged in a counterclockwise direction. Although the present disclosure illustrates the clockwise configuration of the slider 102 in FIGS. 2-34, it is noted the orientation and arrangement of either the clockwise slider 102 (e.g., as illustrated in FIGS. 1A and 1B) or the counterclockwise slider 102 (e.g., as illustrated in FIGS. 1C and 1D) are equally preferable and are usable within the settler device 100 as described through the present disclosure.

Referring now to FIGS. 2-7, the settler device 100 with various numbers of sliders 102 is illustrated. The settler device 100 includes a housing or outer structure (e.g., outer cylinder) 200. The sliders 102 fit within a cavity or annular space 204 defined by the housing 200 and an inner cylinder 202 (or inner structure 202). For example, the sliders 102 include an edge 112 which contacts an interior surface 206 of an housing wall 208 of the housing 200, and an edge 114 which contacts an exterior surface 210 of an inner cylindrical wall 212 of the inner cylinder 202. For instance, where the housing 200 is a cylinder or outer cylinder, the housing wall 208 is a cylindrical wall or outer cylindrical wall. By way of another example, the inner cylinder 202 may be hollow (e.g., defining a cavity 214) or solid. The sliders 102 are oriented and arranged at an angle relative to the longitudinal axis of the housing 200 and/or the inner cylinder 202. It is noted, however, the sliders 102 may be generally oriented and arranged so as to be aligned concentrically or be parallel with a longitudinal axis of the housing 200 and/or the inner cylinder 202. It is noted components of the settler device 100 (e.g., including the housing 200 and/or the inner cylinders 202) may be illustrated as being transparent for clarity in FIGS. 1-14, but that one having skill in the art would understand this not to be limiting on the present disclosure.

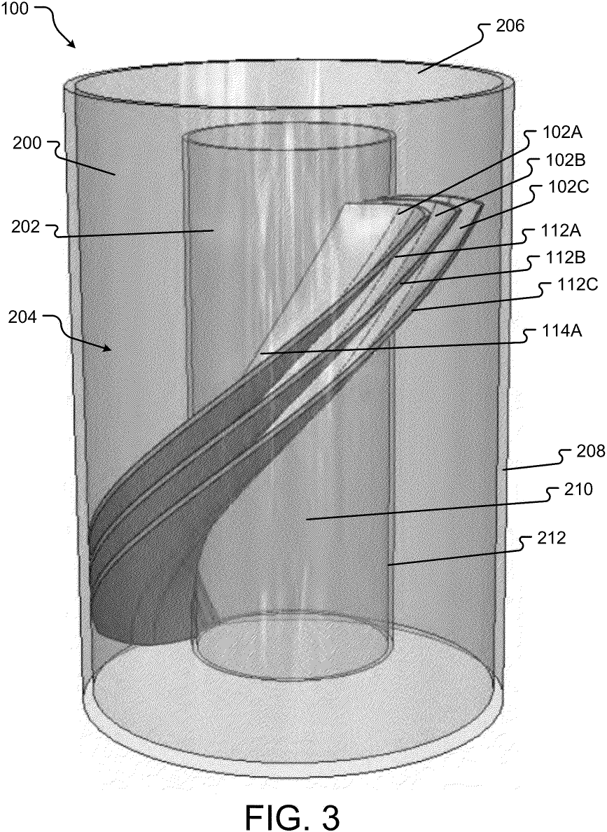

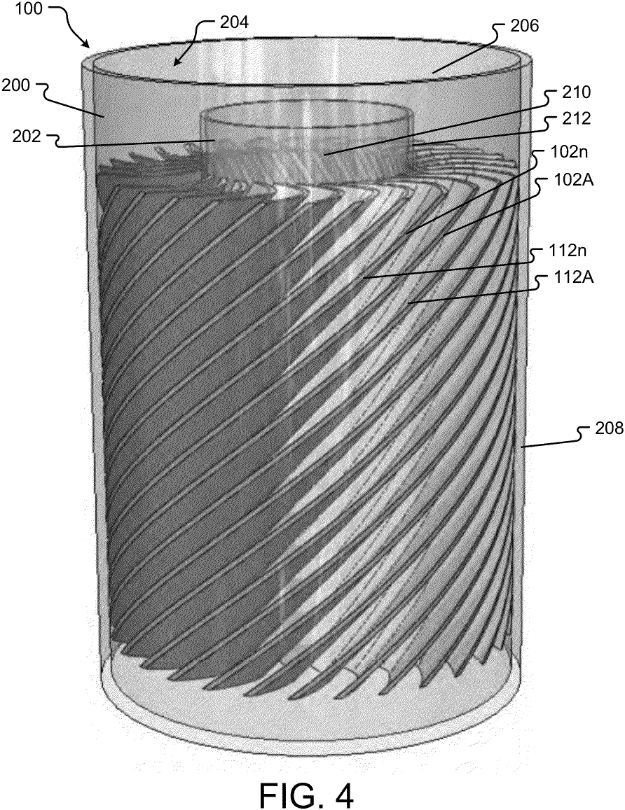

In three example embodiments, the housing 200 includes a single slider 102 as illustrated in FIG. 2, three sliders 102 as illustrated in FIG. 3, and multiple sliders 102 as illustrated in FIGS. 4-7.

In FIG. 2, a single slider 102 is located within the annular space 204 between the housing 200 and the inner cylinder 202. The slider 102 provides a confined inclined settling surface 104 for cells to settle down while protected from the turbulence due to agitation of an impeller for mixing inside a typically cylindrical bioreactor.

In FIG. 3, three sliders 102A-102C are located within the annular space 204 between the housing 200 and the inner cylinder 202. The sliders 102A-102C are fabricated and fixed within the annular space 204 via respective edges 112A-112C and 114A-114C at locations and/or spacings determined through experimentation or computational simulations. For example, the sliders 102 may be 3D printed or injection molded or otherwise attached (e.g., glued, heat welded, or sonically welded) to the housing 200 and/or the inner cylinder 202 at a fixed vertical spacing. For instance, the fixed vertical spacing may range from 1 millimeter (mm) to 20 mm. In one preferable example embodiment the vertical spacing may be 5 mm. By way of another example, the experimentation or computational simulation may compare cells or particles of different sizes ranging from 4 microns (e.g., of a yeast cell) to 15 microns or greater (e.g., for a mammalian cell) to 500 microns (e.g., for microcarrier beads, either with or without adherent cell growing on surfaces). It is noted the vertical spacing may not be fixed (e.g., constant), but instead be variable without departing from the present disclosure. In addition, it is noted the sliders 102 may be coupled or otherwise configured to interact with supports like projections (e.g., spacers, bumps, ridges, flanges, ribs, or the like) upon which the sliders sit or are coupled, or grooves in which the sliders are set.

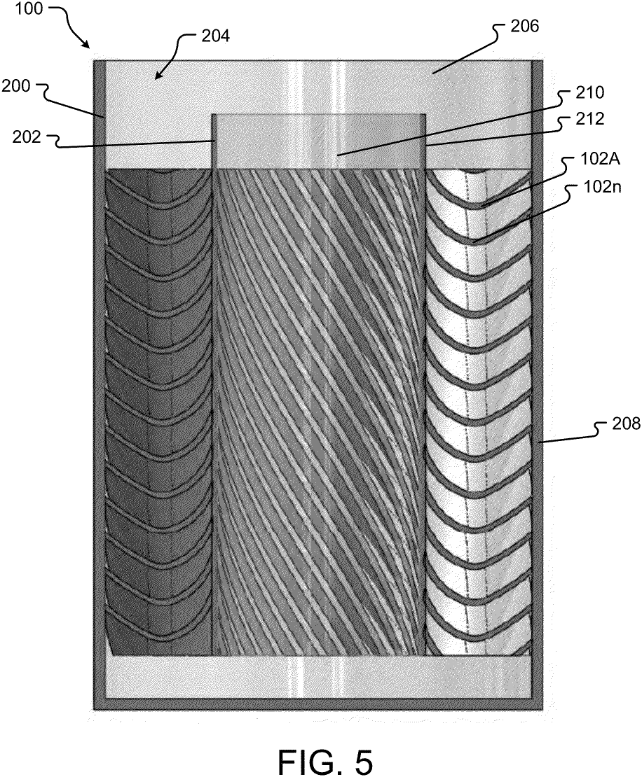

In FIGS. 4-7, multiple sliders 102A-102n, where “n” represents any number of sliders 102, are located within the annular space 204 between the housing 200 and the inner cylinder 202. The sliders 102A-120n are fabricated and fixed within the annular space 204 via respective edges 112A-112n and 114A-114n, where types of fabrication techniques and point of fixation are determined as described with respect to FIG. 3. The n number of sliders 102 may be selected to maintain a relatively constant quiescent or creeping flow velocity profile of liquid moving up or down between the spiral channels. Referring to the cross-section view of FIG. 5, it is noted the curvature of each slider 102 may be symmetrical (e.g., the bottom of each curved slider 102 may be at the center of the annular space 204) or asymmetrical (e.g., skewed closer to the inner cylindrical wall 210, or skewed closer to the housing wall 208). In addition, it is noted the curvature of each cross-section may or may not be a mirror image around a center bottom point. For example, the vertical angle at the inner cylinder 202 may be steeper than the vertical angle at housing 200, as shown in the example embodiments of FIGS. 4-6. It is noted the sliders 102A-102n may have features similar to the sliders 402A-402n, the sliders 1112A-1112n, the sliders 1208A-1208n, and/or the sliders 1308A-1308n as described throughout the present disclosure.

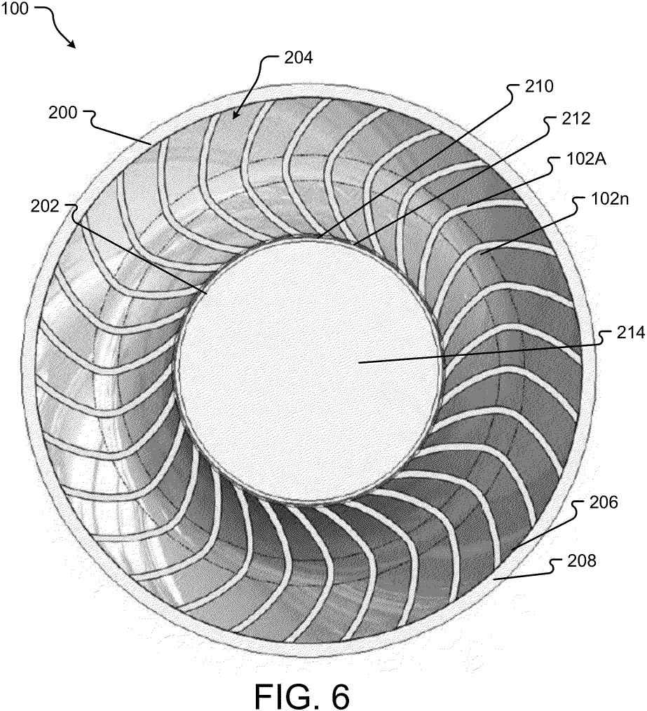

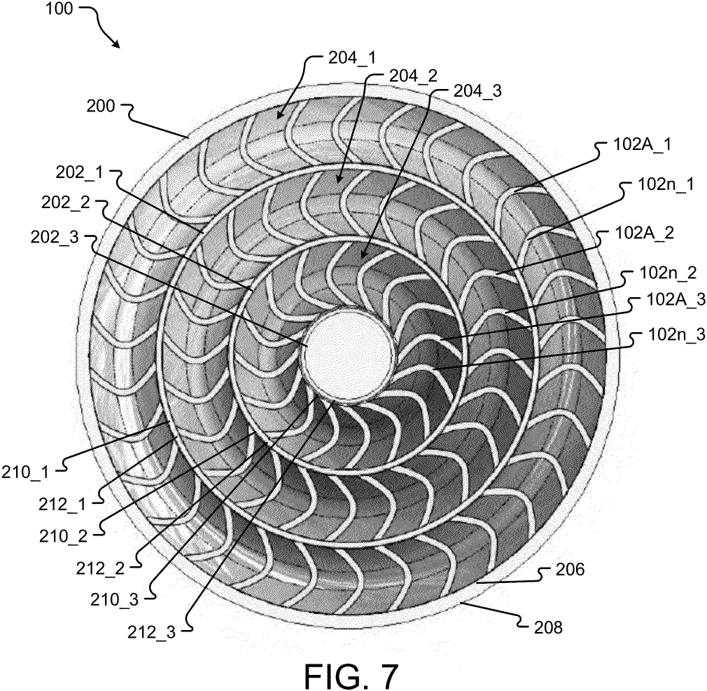

Referring now to FIG. 7, an example embodiment includes concentric inner cylinders 202_1, 202_2, 202_3 of sliders 102A-102n installed within the housing 200. The outer rings will have more numerous spiral sliders 102 compared to the inner rings. Having multiple concentric rings of spiral sliders 102 provides a more uniform, or less variation in, vertical spacing between adjacent spiral sliders 102 within each respective ring. It is noted the present disclosure is not limited to the three concentric rings of sliders 102 as illustrated in FIG. 7, but that the settler device 100 may include any n number of concentric rings of sliders 102 with departing from the scope of the present disclosure.

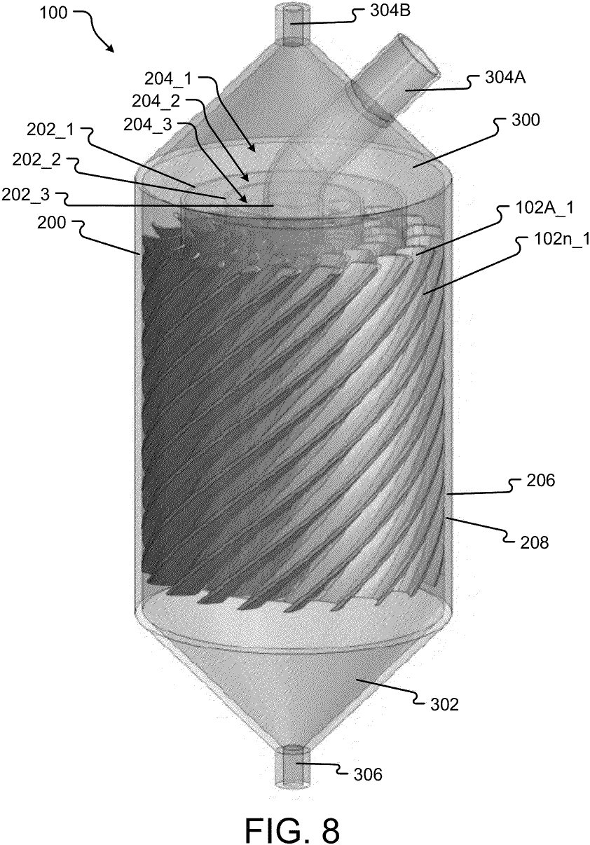

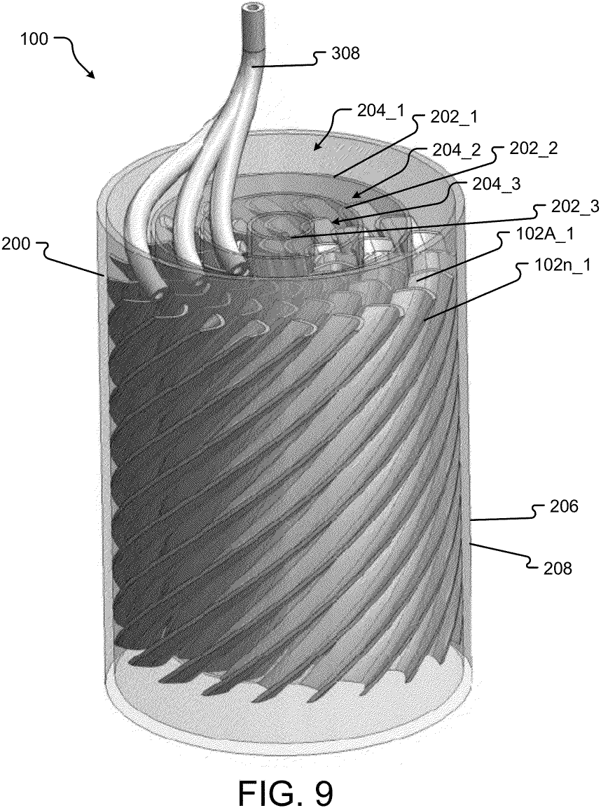

Referring now to FIGS. 8 and 9, the particle settler device 100 with the example embodiment of FIG. 7 is illustrated. The settler device 100 includes a top closure 300 and a bottom closure 302. For example, the top closure 300 and/or the bottom closure 302 may be conical as illustrated in FIG. 8. For instance, the conical top closure 300 and/or the bottom closure 302 may include a body having an apex with a small opening and a base with a large opening. In addition, the conical top closure 300 may have a first end that has a first diameter and a second end with a second diameter that is larger than the first diameter, the first end being oriented toward the conical bottom closure 302. By way of another example, the top closure 300 and/or the bottom closure 302 may be planar or flat.

In one example embodiment, the bottom closure 302 may be a lower section of the settler device 100, with a port 306. The housing 200 may have a lower end contacting and extending upwardly from the bottom closure 302, an upper end, and a housing wall 208. A plurality of inclined sliders 102 may be provided within an annular space 204 defined by an interior surface 206 of the housing wall 208 and an exterior surface 210 of an inner cylindrical wall 212. Each of the plurality of inclined sliders 102 may include a body with an arcuate surface 104 having a groove 106 and sides 108 spaced from a first adjacent inclined slider 102, at least one of an edge 112 coupled to the interior surface 208 of the housing 102 and/or an edge 114 coupled to the exterior surface 212 of the at least one inner structure 104, and a second arcuate surface spaced from a second adjacent inclined slider 102. The top closure 300 may be an upper section of the settler device 100, with at least one port 304.

It is noted the top closure 300 and/or the bottom closure 302 may be coupled (e.g., mechanically, or the like) to the housing 200 via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the lid or top closure or upper portion 300 and/or the bottom closure or lower portion 302 to the housing 200 may be used with the settler device 100. In addition, it is noted a washer or gasket may be positioned between the housing 200 and the top closure 300 and/or the bottom closure 302 during assembly of the settler device 100.

The top closure 300 includes at least one port 304, and/or the bottom closure 302 includes at least one port 306. Optionally, a port 304 and a port 306 may be generally aligned concentrically or be parallel with a longitudinal axis of the housing 200 and/or the inner cylinder 202. In addition, a port 304 and/or a port 306 may be angled relative to the longitudinal axis of the housing 200 and/or the inner cylinder 202. By way of another example, the at least one port 304 may be a single port or separate into multiple ports prior to interaction with the sliders 102, and/or the at least one port 306 may be a single port or rejoin into a single port following interaction with the sliders 102.

In one example embodiment as illustrated in FIG. 8, liquid or fluid containing cells or particles is pumped or forced into the settler device 100 from angled top side port 304A and flows down into the central cylindrical portion of the settler device 100. The outlet fluid flow via the central bottom port 306 is controlled (e.g., by a downstream pump, or the like) at a slower flow rate than the top side inlet flow rate, forcing some of the liquid/fluid to flow up through the concentric rings 202_1, 202_2, 202_3 of sliders 102A-102n. Solid microcarrier particles or semi-solid single cells or cellular aggregates or organoids will settle down on the sliders 102 as the fluid flows slowly up the slides. Settling cells 110 or particles get concentrated by the curvatures of each slider 102 spiraling down the settler device 100 and accumulate into the bottom conical portion 302 and exit via the bottom central port 308.

In another example embodiment as illustrated in FIG. 9, the top closure 300 may lead to curved top effluent tubes. In a slightly more complicated manifestation of a stand-alone settler device 100 in the example embodiment of FIG. 8, clarified supernatant from the top of each concentric ring of sliders 102 can be collected by a separate tube, along with any gas bubbles entering with the liquid in the top side inlet port 304A and removed via the top central port 304B (e.g., as shown in FIG. 8), while the concentrated cells or particles are removed via the bottom central port 306 shown in FIG. 8.

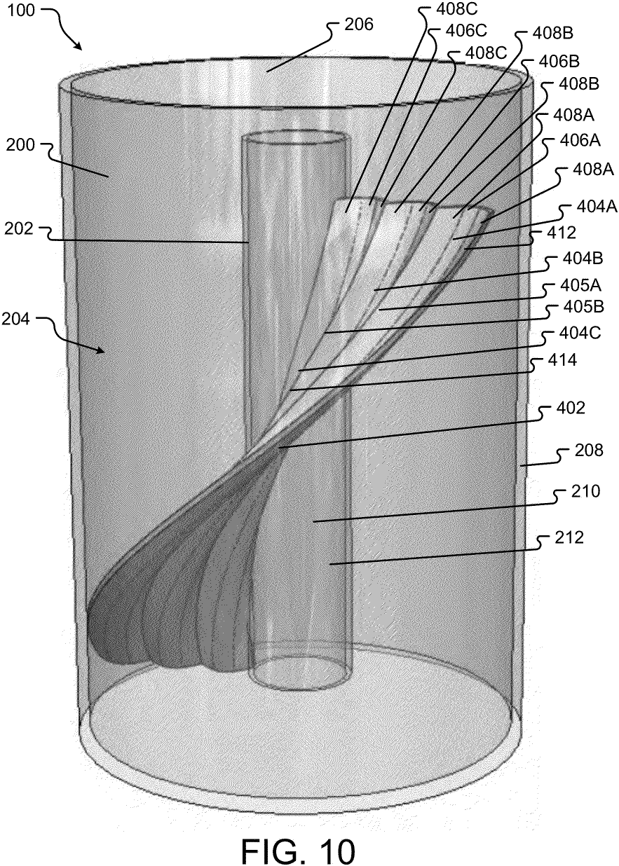

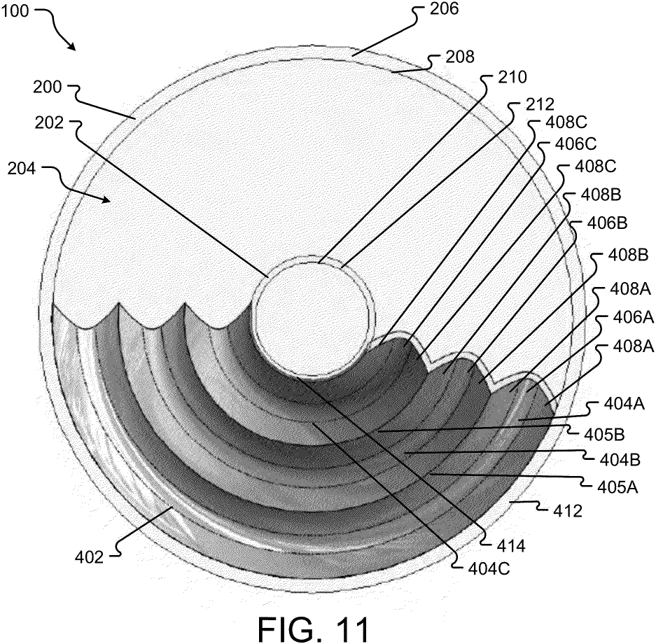

Referring now to FIGS. 10 and 11, an inclined spiral slide settler 402 (or slider 402) of the particle settler device 400 is illustrated. It is noted the slider 402 as described throughout the present disclosure may be incorporated or inserted into any suspension bioreactor, and/or may have features similar to the sliders 102A-102n, the sliders 1112A-1112n, the sliders 1208A-1208n, and/or the sliders 1308A-1308n as described throughout the present disclosure.

The slider 402 includes multiple convex or concave surfaces 404 such that a cross-section of the slider 402 defines an arcuate line, which are separated by a seam 405. The surfaces 404 each include a groove 406 and sides 408. For example, each set may include a groove 406 and corresponding sides 408 which may be an arcuate shape with a constant radius of curvature or multiple radii of curvature. By way of another example, each set may include a groove 406 set at an angle from corresponding sides 408, with the groove 406 and/or the sides 408 forming a curved surface or a planar or flat surface.

The surface 404 directs particles along the length of the slider 402 (e.g., from a higher level of the particle settler device 100 to a lower level of the particle settler device 100). The slider 402 is oriented and arranged within the particular settler device 100 in a particular direction. For example, the slider 402 may be oriented and arranged in a clockwise direction, or oriented and arranged in a counterclockwise direction. The sliders 402 are oriented and arranged at an angle relative to the longitudinal axis of the housing 200 and/or the inner cylinder 202. It is noted, however, the sliders 402 may be generally oriented and arranged so as to be aligned concentrically or be parallel with a longitudinal axis of the housing 200 and/or the inner cylinder 202.

The sliders 402 fit within the annular space 204 defined by the housing 200 and the inner cylinder 202. The sliders 402 include an edge 412 which contacts an interior surface 206 of an housing wall 208 of the housing 200, and an edge 414 which contacts an exterior surface 210 of an inner cylindrical wall 212 of the inner cylinder 202. For example, the sliders 402 may contact the interior surface 206 with the edge 412 and/or the housing wall 208 with the edge 414 at locations and/or spacings determined through experimentation or computational simulations. For example, the sliders 402 may be 3D printed or injection molded or otherwise attached (e.g., glued, heat welded, or sonically welded) to the housing 200 and/or the inner cylinder 202 at a fixed vertical spacing. For instance, the fixed vertical spacing may range from 1 millimeter (mm) to 20 mm. In one preferable example embodiment the vertical spacing may be 5 mm. By way of another example, the experimentation or computational simulation may compare cells or particles of different sizes ranging from 4 microns (e.g., of a yeast cell) to 15 microns or greater (e.g., for a mammalian cell) to 500 microns (e.g., for microcarrier beads, either with or without adherent cell growing on surfaces). It is noted the vertical spacing may not be fixed (e.g., constant), but instead be variable without departing from the present disclosure. In addition, it is noted the sliders 402 may be coupled or otherwise configured to interact with supports like projections (e.g., spacers, bumps, ridges, flanges, ribs, or the like) upon which the sliders sit or are coupled, or grooves in which the sliders are set.

In this regard, larger annular spaces 204 may be filled a single slider 402 with multiple grooves 404 on each, instead of installing multiple cylinders 202 with their respective annular spaces 204 filled with single grooved sliders 102 (e.g., as illustrated in the example embodiment of FIGS. 7-9). Although the present disclosure illustrates the sliders 102 in FIGS. 2-9 and 12-34, it is the sliders 102 (e.g., as illustrated in FIGS. 1A-1D) or the sliders 402 (e.g., as illustrated in FIGS. 10 and 11) are equally preferable and are usable within the settler device 100 as described through the present disclosure. In this regard, although no equivalent sliders 402 versions of FIGS. 2-9 and 12-34 are provided, the lack of figures should not be interpreted as being limiting to the present disclosure.

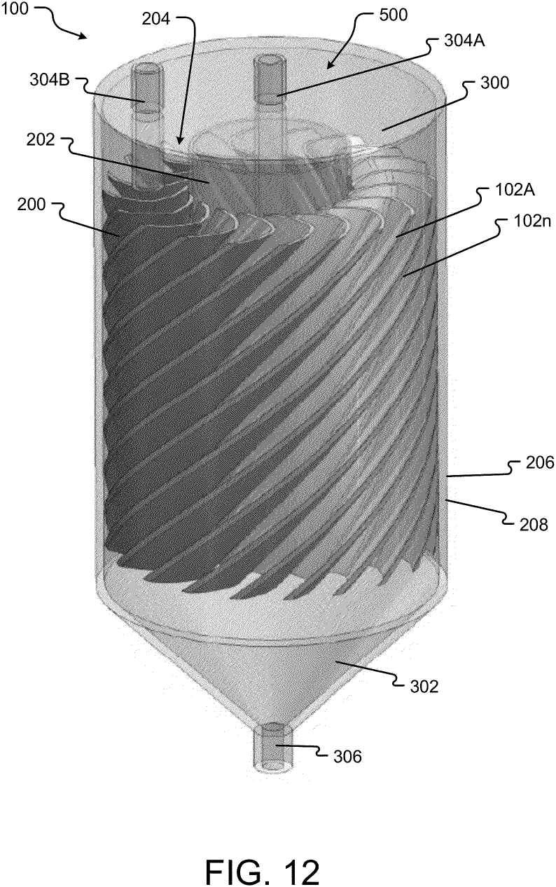

Referring now to FIGS. 12-14, the settler device 100 includes a single ring of single-groove sliders 102 between the housing 200 and the inner cylinder 202. The settler device 100 includes a planar or flat top closure 300 with multiple ports 304, and a conical bottom closure 302 with a port 306.

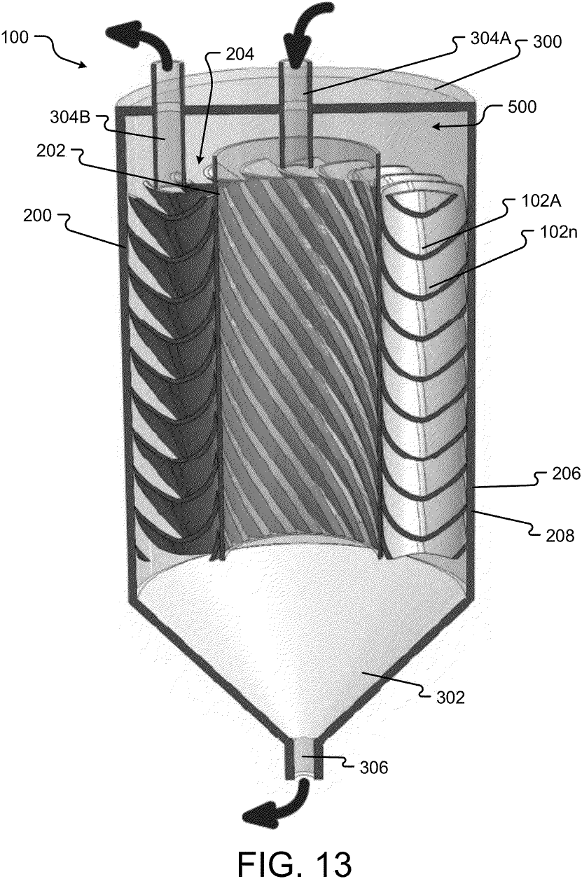

In FIGS. 12 and 13, the settler device 100 may include an inlet port 304A at the top center and two outlet ports, with one outlet carrying the concentrated cell or particle suspension out via the bottom central port 306, and the other outlet carrying the clarified supernatant (e.g., clarified culture fluid containing any metabolic waste products, such as ammonia and lactate, or gasses, along with any not-yet settled smaller dead cells and cell debris) from the top annular space 500 above the annular space 204 housing the sliders 102 via the top port 304B. In particular, FIG. 13 illustrates the input of cell or particle contained fluid via inlet port 304A and the two outlets. The bottom central port 306 may operate as the outlet for the concentrated fluid, and the top side port 304B may operate as the outlet for the relatively clarified or cell-free (or reduced cell concentration) or particle-free (or significantly reduced particle concentration) supernatant.

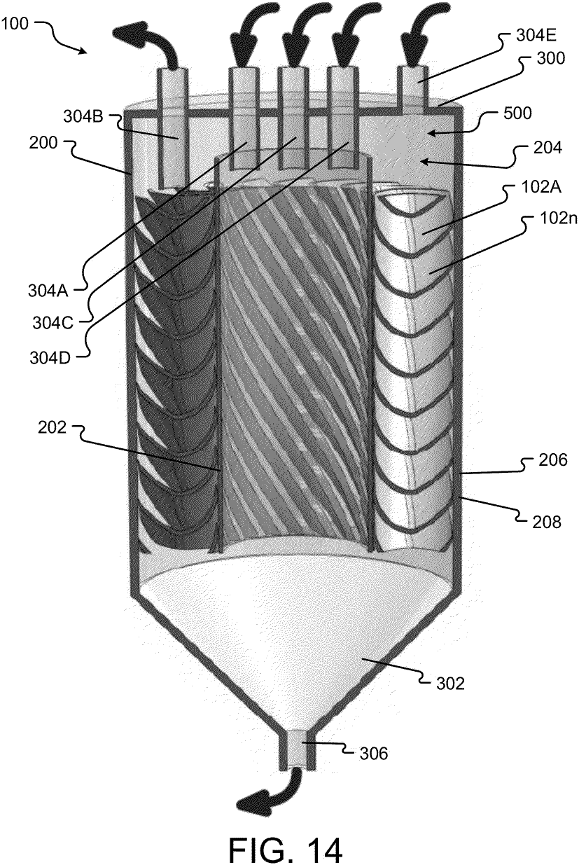

In FIG. 14, the settler device 100 includes a top closure 300 with multiple inlet ports 304A, 304C, 304D and a gas overlay inlet port 304E in addition to the outlet port 304B. Some process applications may need mixing of two or more inlet liquids (for example, acid and/or base along with cell culture broth to facilitate cells to aggregate at lower pH) via the multiple inlet ports 304A, 304C, and 304D. In addition, a controlled mixture of air, O₂, CO₂, and N₂ may also be pumped into the gas overlay port 304E to control the pH and DO of the culture supernatant inside the settler device 100. Such cell aggregates will settle more rapidly and slide down faster on sliders 102 to provide a faster outlet rate of highly clarified supernatant (containing secreted protein products and very few cells).

Elements of the settler device 100, such as the sliders 102, 402 and the housing 200 and inner cylinders 202, may be fabricated of a single-use, disposable plastic. Alternatively, one or more of the elements of the settler device 100 may be manufactured of a metal, such as a stainless-steel alloy, or glass. Depending on the fabrication material, at least a portion of the elements of the settler device 100 may be partially or fully transparent or at least translucent to light of a predetermined range or ranges of wavelengths.

Surfaces within the settler device 100, such as the surfaces 104, 404, the interior surface 206 of the housing wall 208, and the exterior surface 210 of the inner cylinder wall 212, may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The settler devices 100 may be easily scaled to any desired size.

It is noted one or more of the ports 304, 306 may be configured to couple (e.g., mechanically, fluidically, or the like) to external tubing lines. Such tubing line may be interconnected to any of the compact cell settler devices of the present disclosure. The line may have a diameter or otherwise be configured to interconnect to any port of embodiments of the present disclosure. Optionally, one or more of the ports 304, 306 may be used for sampling bioreactor contents, for example to check cell viability, and continuous measurement of liquid pH and DO for inputs into a computer-controlled multi-gas mass flow controller. The line may optionally include at least one sensor positioned within a hollow interior. The sensors may be in contact with fluid and/or particles within the line. Optionally, the sensors may be arranged on an interior surface of the line, although other configurations are contemplated. The sensors may be operable to monitor one or more of pH, DO, glucose, temperature, and CO₂ (including dissolved or partial CO₂) in the line. Optionally, one or more of the sensors may comprise a fluorescent probe which emits light that varies based on a condition sensed by the probe. The light may be collected by a reader or meter. Optionally, the light may be collected by an optional fiber cable and transmitted to the meter. The meter is operable to report or display levels of at least one of pH, DO, glucose, temperature, and CO₂ sensed by the fluorescent probes. The tubing line may comprise a material that is transparent or at least translucent. Thus, light generated by a sensor may pass through the line. Alternatively, at least a portion of a line is transparent or at least translucent, similar to a window. Accordingly, light generated by a sensor may be transmitted through window portion and collected by the meter.

The housing 200 and/or the inner cylinders 202 may optionally include a fluid jacket (not illustrated). The fluid jacket can operate such that water or other fluids may be directed into the fluid jacket through one or more ports to maintain the housing 200 and/or the inner cylinders 202 and contents within the settler device 100 within a desired temperature range. Optionally, a heater may be connected to one or more of the housing 200 and/or the inner cylinders 202 to adjust the temperature of liquid within the settler 100. For example, in one embodiment, a heat exchanger to heat or cool the inlet cell culture media can be connected to the ports 304, 306.

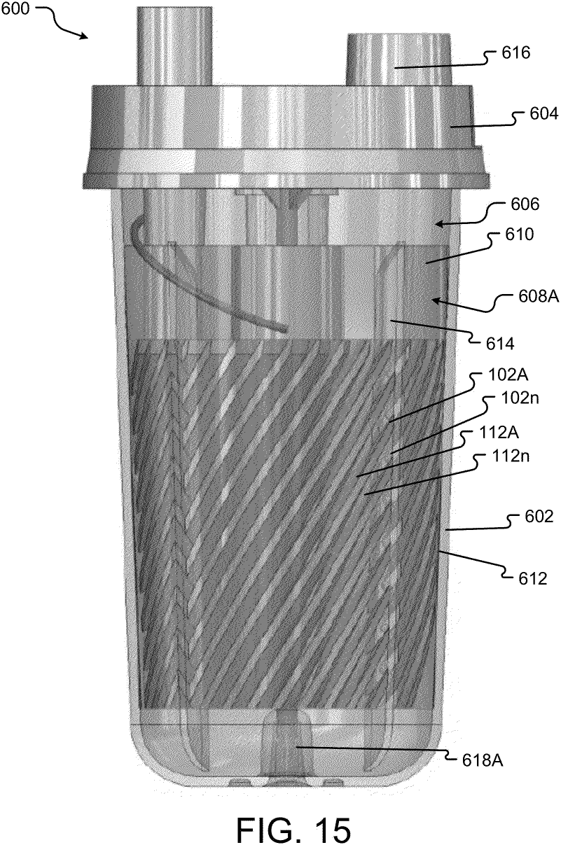

FIGS. 15-22 in general illustrate a bioreactor 600 according to embodiments of the present disclosure. It is noted portions of the bioreactor 600 (e.g., the housing 602 and/or the lid or top closure 604 including ports 616, 618 as well as internal components such as, but not limited to, the impeller 620) in FIGS. 15-20 are example representations of components of the Ambr® 250 microcarrier and/or mammalian vessel manufactured and/or marketed by Sartorius as part of their multiplexed bioreactors robotically controlled in a biosafety cabinet. The representations are provided as an example that the sliders 102 are configurable for use with components of the Ambr® 250 microcarrier and/or mammalian vessel.

In addition, embodiments directed to the settler device 100, the bioreactor 700, the bioreactor 800, the bioreactor 1000, the bioreactor 1100, the biosettler 1200, and/or the bioreactor 1300 may be applied to the bioreactor 600 unless otherwise noted. In particular, the bioreactor 600 is similar to other settler devices or bioreactors described herein and includes many of the same or similar features.

The bioreactor 600 includes a housing 602 and a lid or top closure 604 which define a cavity 606. For example, the top closure 604 and/or a bottom closure may be planar or flat. By way of another example, the top closure 604 and/or the bottom closure may be conical, hemispherical, semispherical or dome shaped, or other three-dimensional shape known in the art. It is noted herein the top closure 604 and/or the bottom closure may be formed as part of the housing 602. It is noted components of the bioreactor 600 (e.g., including the housing 602) may be illustrated as being transparent for clarity in FIGS. 15-22, but that one having skill in the art would understand this not to be limiting on the present disclosure.

The housing 602 is configured to receive multiple sliders 102A-102n as described throughout the present disclosure. The sliders 102A-102n are installed within a cavity portion 608A of the cavity 606. The cavity portion 608A is separated from a cavity portion 608B of the cavity 606 via a separation plate 610 (or inner structure 610). For example, the separation plate 610 may be generally aligned or be parallel with a longitudinal axis of the housing 602.

The sliders 102A-102n are fabricated and fixed within the cavity portion 608A via edges 112A-112n and 114A-114nat locations and/or spacings determined through experimentation or computational simulations. For example, the sliders 102A-102n may be 3D printed or injection molded or otherwise attached (e.g., glued, heat welded, or sonically welded) to an interior surface 612 of the housing 602 and/or a surface 614 of the separation plate 610. It is noted the interior surface 612 may include alignment fins or brackets 614 configured to guide the sliders when installed in the housing and/or prevent the sliders 102A-102n from moving within the cavity portion 608A.

The bioreactor 600 includes one or more inlet ports 616 within the top closure 604 and one or more outlet ports 618A within the housing 602 and/or outlet ports 618B within the top closure 604. For example, cell or particle contained fluid, multiple inlet fluids to be mixed, and/or overlay gas may be input into the housing 602 via the one or more inlet ports 616, while concentrated cell or particle suspension and/or clarified supernatant (e.g., clarified culture fluid containing any metabolic waste products, such as ammonia and lactate, or gasses, along with any not-yet settled smaller dead cells and cell debris) may be output from the housing 602 via the one or more outlet ports 618A and/or from the top closure 604 via the one or more outlet ports 618B.

The bioreactor 600 includes an impeller 620 with one or more sets of blades 622 installed within the cavity portion 608B. The impeller 620 is rotated by a power transmission device 624. For example, the power transmission device 624 may be an electric motor, a turbine, an induction device, or another device capable of being driven by electricity, fluid, or magnetic forces. The impeller 620 agitates fluid and cause microcarrier particles or semi-solid single cells or cellular aggregates or organoids to settle down on the sliders 102A-102n. It is noted the bioreactor 600 may include other outlets for carrying the clarified supernatant (e.g., clarified culture fluid containing any metabolic waste products, such as ammonia and lactate, or gasses, along with any not-yet settled smaller dead cells and cell debris) out of the housing 602.

The housing 602 includes a lip or flange 626 which interfaces with the top 604. It is noted the top closure 604 may be coupled (e.g., mechanically, or the like) to the housing 602 via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). For example, the top closure 604 may include a mated component to the flange 626, or a protrusion or groove on or proximate to the flange 626. As will be appreciated by one of skill in the art, any suitable means of joining the lid or top closure or upper portion 604 and/or a bottom closure or lower portion to the housing 602 may be used with the bioreactor 600. In addition, it is noted a washer or gasket may be positioned on the flange 626 or within a groove in the flange 626 (not shown) between the housing 602 and the top closure 604 during assembly of the bioreactor 600.

In one example embodiment, the separation plate 610 may be contoured. The separation plate 610 may include one or more arcuate shapes. The arcuate shapes may include a primary arcuate shape 628 and secondary arcuate shapes 630A, 630B. For example, the primary arcuate shape 628 and secondary arcuate shapes 630A, 630B may be an arcuate shape with a constant radius of curvature or multiple radii of curvature. By way of another example, the primary arcuate shape 628 and secondary arcuate shapes 630A, 630B may include a center portion set at an angle from side portions, with the center portion and/or the side portions being a curved surface or a planar or flat surface.

In another example embodiment, the separation plate 610 may include one or more indentation or grooves 632 which are configured to accept a corresponding set of blades 622 of the impeller 620. In this regard, the bioreactor 600 may be more compact design, requiring less material and space than known bioreactors. In addition, the compact design may facilitate the separation plate 610 and sliders 102 to be retrofitted into known bioreactors. It is noted that the separation plate 610 with sliders 102A-102n may be considered a settling assembly 634, for purposes of the present disclosure.

Elements of the bioreactor 600, such as the sliders 102 and the housing 602 and separation plate 610, may be fabricated of a single-use, disposable plastic. Alternatively, one or more of the elements of the bioreactor 600 may be manufactured of a metal, such as a stainless-steel alloy, or glass. Depending on the fabrication material, at least a portion of the elements of the bioreactor 600 may be partially or fully transparent or at least translucent to light of a predetermined range or ranges of wavelengths.

Surfaces within the bioreactor 600, such as the surfaces 104 of the sliders 102, the interior surface 612 of the housing 602, and the exterior surface 614 of the separation plate 610, may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The bioreactor 600 may be easily scaled to any desired size.

It is noted one or more of the ports 616, 618 may be configured to couple (e.g., mechanically, fluidically, or the like) to external tubing lines. Such tubing line may be interconnected to any of the compact cell settler devices of the present disclosure. The line may have a diameter or otherwise be configured to interconnect to any port of embodiments of the present disclosure. Optionally, one or more of the ports 616, 618 may be used for sampling bioreactor contents, for example to check cell viability, and continuous measurement of liquid pH and DO for inputs into a computer-controlled multi-gas mass flow controller. The line may optionally include at least one sensor positioned within a hollow interior. The sensors may be in contact with fluid and/or particles within the line. Optionally, the sensors may be arranged on an interior surface of the line, although other configurations are contemplated. The sensors may be operable to monitor one or more of pH, DO, glucose, temperature, and CO₂ (including dissolved or partial CO₂) in the line. Optionally, one or more of the sensors may comprise a fluorescent probe which emits light that varies based on a condition sensed by the probe. The light may be collected by a reader or meter. Optionally, the light may be collected by an optional fiber cable and transmitted to the meter. The meter is operable to report or display levels of at least one of pH, DO, glucose, temperature, and CO₂ sensed by the fluorescent probes. The tubing line may comprise a material that is transparent or at least translucent. Thus, light generated by a sensor may pass through the line. Alternatively, at least a portion of a line is transparent or at least translucent, similar to a window. Accordingly, light generated by a sensor may be transmitted through window portion and collected by the meter.

The housing 602 and/or the separation plate 610 may optionally include a fluid jacket (not illustrated). The fluid jacket can operate such that water or other fluids may be directed into the fluid jacket through one or more ports to maintain the housing 602 and/or the separation plate 610 and contents within the bioreactor 600 within a desired temperature range. Optionally, a heater may be connected to one or more of the housing 602 and/or the separation plate 610 to adjust the temperature of liquid within the bioreactor 600. For example, in one embodiment, a heat exchanger to heat or cool the inlet cell culture media can be connected to the ports 616, 618.

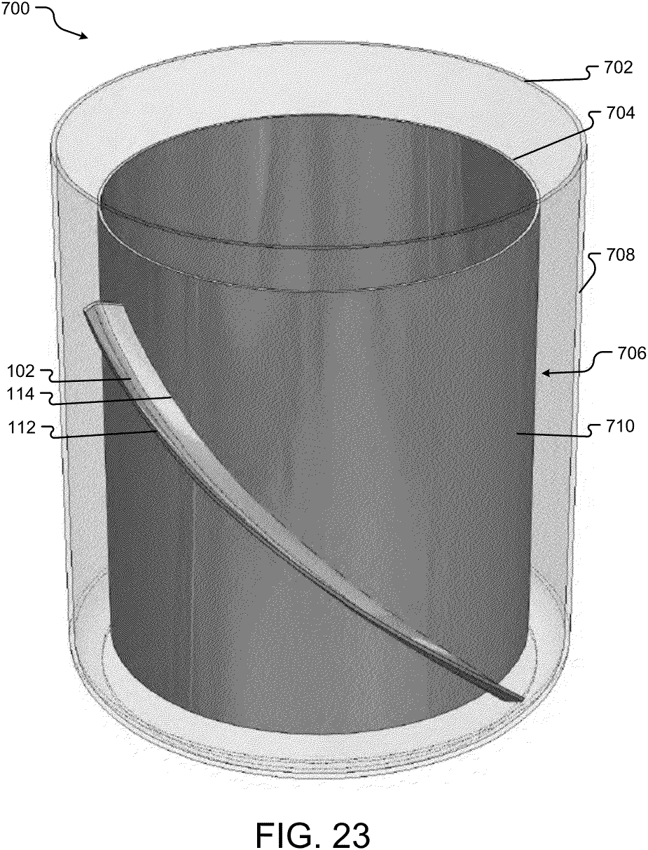

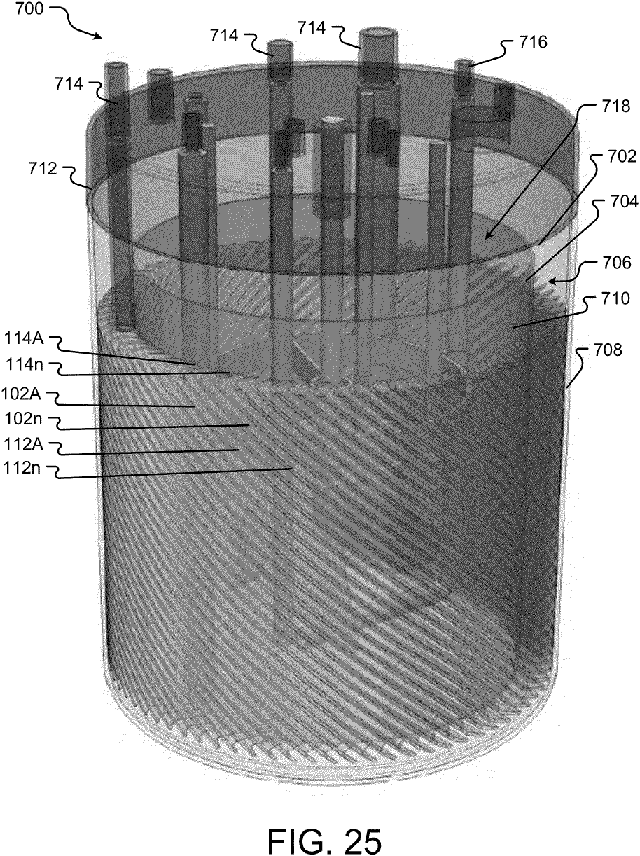

FIGS. 23-28 in general illustrate a bioreactor 700 according to embodiments of the present disclosure. It is noted portions of the bioreactor 700 (e.g., the lid or top closure 712 including ports 714, 716 as well as internal components such as, but not limited to, the impeller 720) in FIGS. 25-28 are example representations of components of the BioBLU® single-use bioreactor manufactured and/or marketed by Eppendorf. The representations are provided as an example that the housing 702 and the installed sliders 102 (e.g., being 3D-printed or otherwise fabricated as described throughout the disclosure) are configurable for use with components of the BioBLU® single-use bioreactor.

In addition, embodiments directed to the settler device 100, the bioreactor 600, the bioreactor 800, the bioreactor 1000, the bioreactor 1100, the biosettler 1200, and/or the bioreactor 1300 may be applied to the bioreactor 700 unless otherwise noted. In particular, the bioreactor 700 is similar to other settler devices or bioreactors described herein and includes many of the same or similar features.

The bioreactor 700 includes a housing 702 and an inner cylinder 704 which define an annular space 706. For example, the housing 702 may be an outer cylinder 702, and the inner cylinder 704 and the outer cylinder 702 may be generally aligned or share a longitudinal axis. The annular space 706 is configured to receive multiple sliders 102A-102n as described throughout the present disclosure. It is noted components of the bioreactor 700 (e.g., including the housing 702 and/or the inner cylinder 704) may be illustrated as transparent for clarity in FIGS. 23-28, but that one having skill in the art would understand this not to be limiting on the present disclosure.

The sliders 102A-102n are fabricated and fixed within the annular space 706 via edges 112A-112n and 114A-114n at locations and/or spacings determined through experimentation or computational simulations. For example, the sliders 102A-102n may be 3D printed or injection molded or otherwise attached (e.g., glued, heat welded, or sonically welded) to an interior surface 708 of the housing 702 and/or an exterior surface 710 of the inner cylinder 704.

The bioreactor 700 includes a lid or top closure 712 and a bottom closure. For example, the top closure 814 and/or the bottom closure may be planar or flat. By way of another example, the top closure 814 and/or the bottom closure may be conical, hemispherical, semispherical or dome shaped, or other three-dimensional shape known in the art. It is noted herein the top closure and/or the bottom closure may be formed as part of the housing 702.

The bioreactor 700 includes one or more inlet ports 714 and one or more outlet ports 716 within the top closure 712, the housing 702, and/or the inner cylinder 704. For example, cell or particle contained fluid, multiple inlet fluids to be mixed, and/or overlay gas may be input via the one or more inlet ports 714, while concentrated cell or particle suspension and/or clarified supernatant (e.g., clarified culture fluid containing any metabolic waste products, such as ammonia and lactate, or gasses, along with any not-yet settled smaller dead cells and cell debris) may be output from the via the one or more outlet ports 716.

It is noted the top closure 712 may be coupled (e.g., mechanically, or the like) to the housing 702 via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the lid or top closure or upper portion 712 and/or the bottom closure or lower portion to the housing 702 may be used with the bioreactor 700. In addition, it is noted a washer or gasket may be positioned on the housing 702 or within a groove in a rim or flange of the housing 702 (not shown) between the housing 702 and the top closure 712 during assembly of the bioreactor 700.

The inner cylinder 704 is hollow and defines an inner annular space 718. The bioreactor 700 includes an impeller 720 with one or more sets of blades 722 installed within the inner annular space 718. The impeller 720 is rotated by a power transmission device. For example, the power transmission device may be an electric motor, a turbine, an induction device, or another device capable of being driven by electricity, fluid, or magnetic forces. The impeller 720 agitates fluid and cause microcarrier particles or semi-solid single cells or cellular aggregates or organoids to settle down on the sliders 102A-102n. It is noted the bioreactor 700 may include other outlets for carrying the clarified supernatant (e.g., clarified culture fluid containing any metabolic waste products, such as ammonia and lactate, or gasses, along with any not-yet settled smaller dead cells and cell debris) out of the housing 702.

Elements of the bioreactor 700, such as the sliders 102 and the housing 702 and inner cylinders 704, may be fabricated of a single-use, disposable plastic. Alternatively, one or more of the elements of the bioreactor 700 may be manufactured of a metal, such as a stainless-steel alloy, or glass. Depending on the fabrication material, at least a portion of the elements of the bioreactor 700 may be partially or fully transparent or at least translucent to light of a predetermined range or ranges of wavelengths.

Surfaces within the bioreactor 700, such as the surfaces 104 of the sliders 102, the interior surface 708 of the housing 702, and the exterior surface 710 of the inner cylinders 704, may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The bioreactor 600 may be easily scaled to any desired size.

It is noted one or more of the ports 714, 716 may be configured to couple (e.g., mechanically, fluidically, or the like) to external tubing lines. Such tubing line may be interconnected to any of the compact cell settler devices of the present disclosure. The line may have a diameter or otherwise be configured to interconnect to any port of embodiments of the present disclosure. Optionally, one or more of the ports 714, 716 may be used for sampling bioreactor contents, for example to check cell viability, and continuous measurement of liquid pH and DO for inputs into a computer-controlled multi-gas mass flow controller. The line may optionally include at least one sensor positioned within a hollow interior. The sensors may be in contact with fluid and/or particles within the line. Optionally, the sensors may be arranged on an interior surface of the line, although other configurations are contemplated. The sensors may be operable to monitor one or more of pH, DO, glucose, temperature, and CO₂ (including dissolved or partial CO₂) in the line. Optionally, one or more of the sensors may comprise a fluorescent probe which emits light that varies based on a condition sensed by the probe. The light may be collected by a reader or meter. Optionally, the light may be collected by an optional fiber cable and transmitted to the meter. The meter is operable to report or display levels of at least one of pH, DO, glucose, temperature, and CO₂ sensed by the fluorescent probes. The tubing line may comprise a material that is transparent or at least translucent. Thus, light generated by a sensor may pass through the line. Alternatively, at least a portion of a line is transparent or at least translucent, similar to a window. Accordingly, light generated by a sensor may be transmitted through window portion and collected by the meter.

The housing 702 and/or the inner cylinder 704 may optionally include a fluid jacket (not illustrated). The fluid jacket can operate such that water or other fluids may be directed into the fluid jacket through one or more ports to maintain the housing 702 and/or the inner cylinder 704 and contents within the bioreactor 700 within a desired temperature range. Optionally, a heater may be connected to one or more of the housing 702 and/or the inner cylinders 704 to adjust the temperature of liquid within the bioreactor 700. For example, in one embodiment, a heat exchanger to heat or cool the inlet cell culture media can be connected to the ports 714, 716.

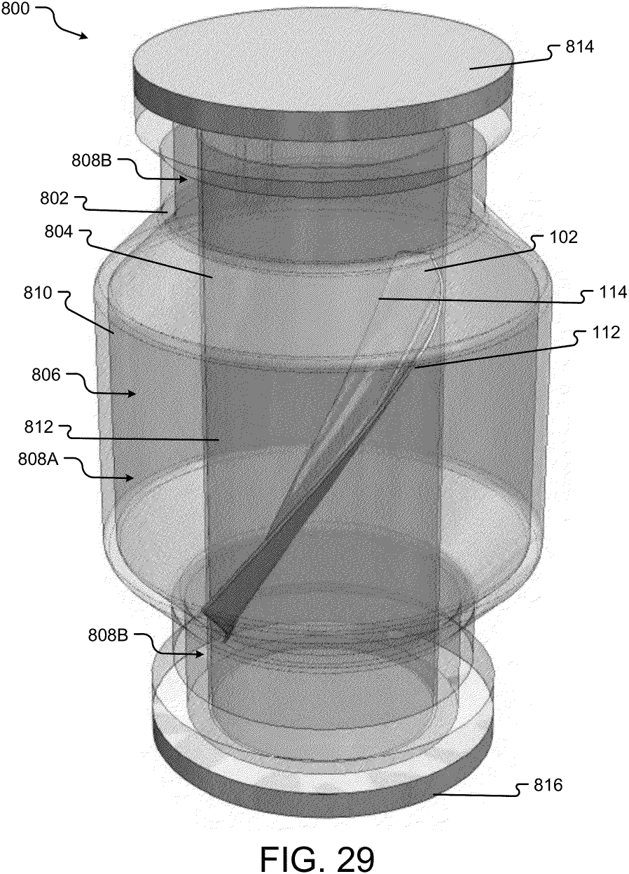

FIGS. 29-34 in general illustrate a bioreactor 800 according to embodiments of the present disclosure. It is noted portions of the bioreactor 800 (e.g., the housing 802, the lid or top closure 814, and/or the bottom closure 816) in FIGS. 29-31 are example representations of components of the KLF bioreactor manufactured and/or marketed by Bioengineering AG. The representations are provided as an example that the sliders 102 and the housing 802, the lid or top closure 814, and/or the bottom closure 816 (e.g., being 3D-printed or otherwise fabricated as described throughout the disclosure) are configurable to be swappable for the glass vessel of the KLF bioreactor and usable with internal components of the KLF bioreactor (not shown in the figures for purposes of clarity).

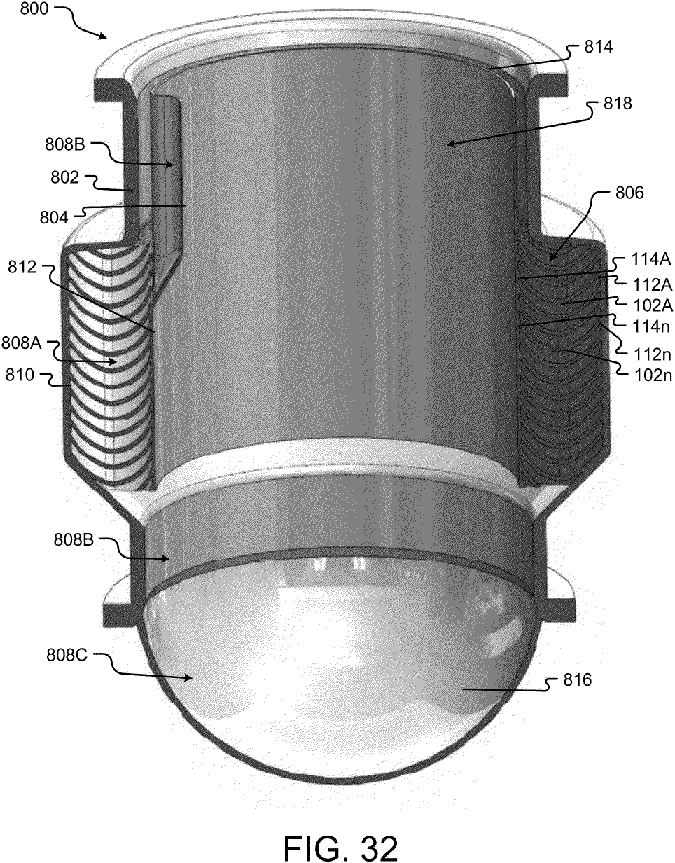

In addition, it is noted portions of the bioreactor 800 (e.g., the housing 802) in FIG. 32 is an example representation of components of the BioFlo® / CelliGen® line of bioreactors manufactured and/or marketed by Eppendorf. The representations are provided as an example that the sliders 102 and the housing 802 (e.g., being 3D-printed or otherwise fabricated as described throughout the disclosure) are configurable to be swappable for the glass vessel of the KLF bioreactor and usable with a shaped (e.g., hemispherical) bottom portion of the BioFlo® / CelliGen® line of bioreactors.

Further, embodiments directed to the settler device 100, the bioreactor 600, the bioreactor 700, the bioreactor 1000, the bioreactor 1100, the biosettler 1200, and/or the bioreactor 1300 may be applied to the bioreactor 800 unless otherwise noted. In particular, the bioreactor 800 is similar to other settler devices or bioreactors described herein and includes many of the same or similar features.

The bioreactor 800 includes a housing 802 and an inner cylinder 804 which define a cavity 806. For example, the inner cylinder 804 and the housing 802 may be generally aligned or share a longitudinal axis.

In some embodiments, the inner cylinder 804 includes a different cross-section than the housing 802. For example, the inner cylinder 804 may include a constant outer diameter, while the housing 802 includes a first, larger diameter proximate to the latitudinal center of the housing 802, and at least a second, smaller diameter proximate to an end of the housing 802. Due to the difference in cross-sections, the cavity 806 includes a cavity portion 808A and at least one cavity portion 808B. The cavity portion 808A is configured to receive multiple sliders 102A-102n as described throughout the present disclosure. In other embodiments, the inner cylinder 804 includes a similar cross-section to the housing 802, without departing from the scope of the present disclosure.

It is noted components of the bioreactor 800 (e.g., including the housing 802 and/or the inner cylinder 804) may be illustrated as transparent for clarity in FIGS. 29-34, but that one having skill in the art would understand this not to be limiting on the present disclosure.

The sliders 102A-102n are fabricated and fixed within the cavity portion 808A via edges 112A-112n and 114A-114n at locations and/or spacings determined through experimentation or computational simulations. For example, the sliders 102A-102n may be 3D printed or injection molded or otherwise attached (e.g., glued, heat welded, or sonically welded) to an interior surface 810 of the housing 802 and/or an exterior surface 812 of the inner cylinder 804.

The bioreactor 800 includes a lid or top closure 814 and a bottom closure 816. For example, the top closure 814 and/or the bottom closure 816 may be planar or flat. By way of another example, the top closure 814 and/or the bottom closure 816 may be conical, hemispherical, semispherical or dome shaped, or other three-dimensional shape known in the art. It is noted herein the top closure 814 and/or the bottom closure 816 may be separate from or formed as part of the housing 802.

The bioreactor 800 includes one or more inlet ports and one or more outlet ports within the housing 802, the inner cylinder 804, the top closure 814, and/or the bottom closure 816. For example, cell or particle contained fluid, multiple inlet fluids to be mixed, and/or overlay gas may be input via the one or more inlet ports, while concentrated cell or particle suspension and/or clarified supernatant (e.g., clarified culture fluid containing any metabolic waste products, such as ammonia and lactate, or gasses, along with any not-yet settled smaller dead cells and cell debris) may be output from the via the one or more outlet ports.

It is noted the top closure 814 and/or the bottom closure 816 may be coupled (e.g., mechanically, or the like) to the housing 802 via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the lid or top closure or upper portion 814 and/or the bottom closure or lower portion 816 to the housing 802 may be used with the bioreactor 800. In addition, it is noted a washer or gasket may be positioned on the housing 802 or within a groove in a rim or flange of the housing 802 (not shown) between the housing 802 and the top closure 814 and/or the bottom closure 816 during assembly of the bioreactor 800.

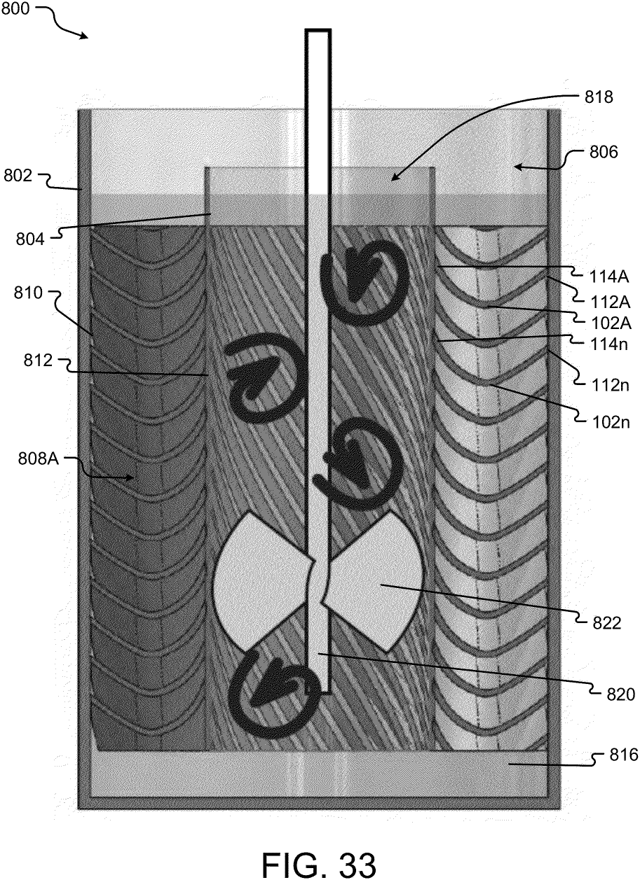

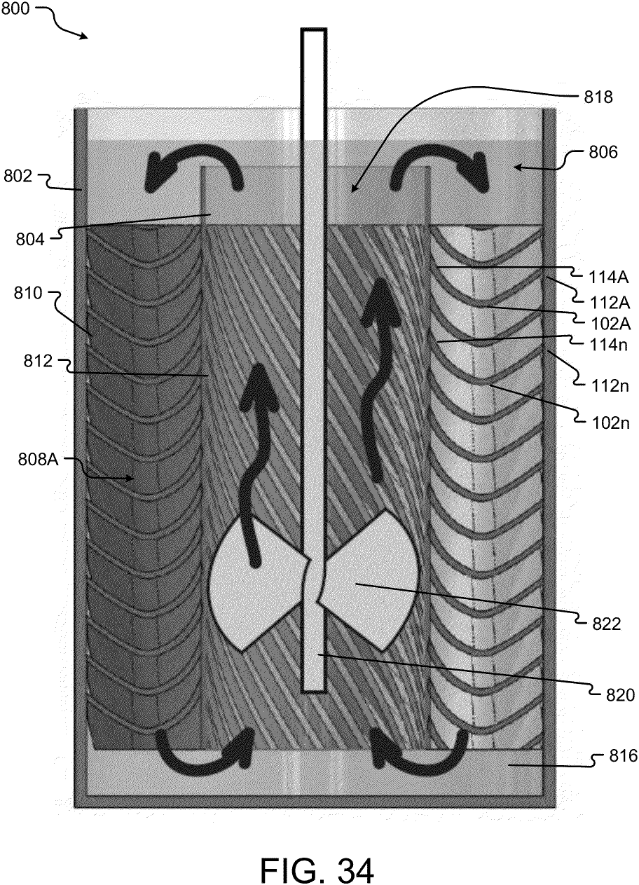

The inner cylinder 804 is hollow and defines an inner cavity 818. The bioreactor 800 includes an impeller 820 with one or more sets of blades 822 installed within the inner cavity 818. The impeller 820 is rotated by a power transmission device. For example, the power transmission device may be an electric motor, a turbine, an induction device, or another device capable of being driven by electricity, fluid, or magnetic forces. As illustrated in FIGS. 33 and 34, the impeller 820 agitates fluid during either isolation or incorporation and causes microcarrier particles or semi-solid single cells or cellular aggregates or organoids to settle down on the sliders 102A-102n during incorporation. For example, the inner cylinder 804 may include one or more openings 824 to facilitate the fluid to flow between the inner cavity 818 and the cavity 806. It is noted the bioreactor 800 may include other outlets for carrying the clarified supernatant (e.g., clarified culture fluid containing any metabolic waste products, such as ammonia and lactate, or gasses, along with any not-yet settled smaller dead cells and cell debris) out of the housing 802.

Elements of the bioreactor 800, such as the sliders 102 and the housing 802 and inner cylinder 804, may be fabricated of a single-use, disposable plastic. Alternatively, one or more of the elements of the bioreactor 800 may be manufactured of a metal, such as a stainless-steel alloy, or glass. Depending on the fabrication material, at least a portion of the elements of the bioreactor 800 may be partially or fully transparent or at least translucent to light of a predetermined range or ranges of wavelengths.

Surfaces within the bioreactor 800, such as the surfaces 104 of the sliders 102, the interior surface 810 of the housing 802, and the exterior surface 812 of the inner cylinders 804, may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The bioreactor 800 may be easily scaled to any desired size.

It is noted one or more of the ports of the bioreactor 800 may be configured to couple (e.g., mechanically, fluidically, or the like) to external tubing lines. Such tubing line may be interconnected to any of the compact cell settler devices of the present disclosure. The line may have a diameter or otherwise be configured to interconnect to any port of embodiments of the present disclosure. Optionally, one or more of the ports of the bioreactor 800 may be used for sampling bioreactor contents, for example to check cell viability, and continuous measurement of liquid pH and DO for inputs into a computer-controlled multi-gas mass flow controller. The line may optionally include at least one sensor positioned within a hollow interior. The sensors may be in contact with fluid and/or particles within the line. Optionally, the sensors may be arranged on an interior surface of the line, although other configurations are contemplated. The sensors may be operable to monitor one or more of pH, DO, glucose, temperature, and CO₂ (including dissolved or partial CO₂) in the line. Optionally, one or more of the sensors may comprise a fluorescent probe which emits light that varies based on a condition sensed by the probe. The light may be collected by a reader or meter. Optionally, the light may be collected by an optional fiber cable and transmitted to the meter. The meter is operable to report or display levels of at least one of pH, DO, glucose, temperature, and CO₂ sensed by the fluorescent probes. The tubing line may comprise a material that is transparent or at least translucent. Thus, light generated by a sensor may pass through the line. Alternatively, at least a portion of a line is transparent or at least translucent, similar to a window. Accordingly, light generated by a sensor may be transmitted through window portion and collected by the meter.

The housing 802 and/or the inner cylinders 804 may optionally include a fluid jacket (not illustrated). The fluid jacket can operate such that water or other fluids may be directed into the fluid jacket through one or more ports to maintain the housing 802 and/or the inner cylinder 804 and contents within the bioreactor 800 within a desired temperature range. Optionally, a heater may be connected to one or more of the housing 802 and/or the inner cylinders 804 to adjust the temperature of liquid within the bioreactor 800. For example, in one embodiment, a heat exchanger to heat or cool the inlet cell culture media can be connected to the ports.

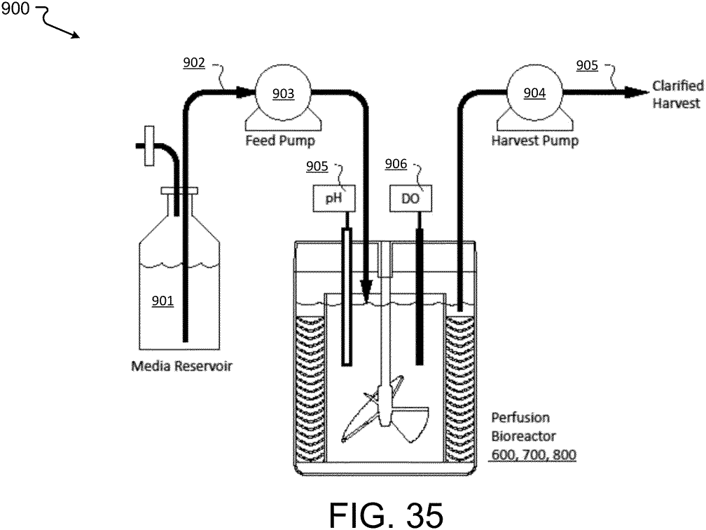

FIG. 35 illustrates an example schematic of a fluid system 900 including a bioreactor 600, 700, 800, 1000, 1100, and/or 1300 according to embodiments of the present disclosure. A bioreactor 600, 700, 800 receives a first media from a reservoir 901 via an input line 902 and a feed pump 903. The bioreactor 600, 700, 800 outputs clarified harvest via an output line 904 and a harvest pump 905. Sensors 906 and 907 read measure at least one of pH, dissolved oxygen (DO), dissolved CO₂, glucose, lactate, glutamine, ammonia, and temperature. Although the sensors 906 and 907 are illustrated as measuring pH and DO in FIG. 35, the labels are not intended to be limiting. In addition, it is noted the layout of the fluid system 900 in FIG. 35 is not intended to be limiting, and that the layout may be altered without departing from the scope of the present disclosure.

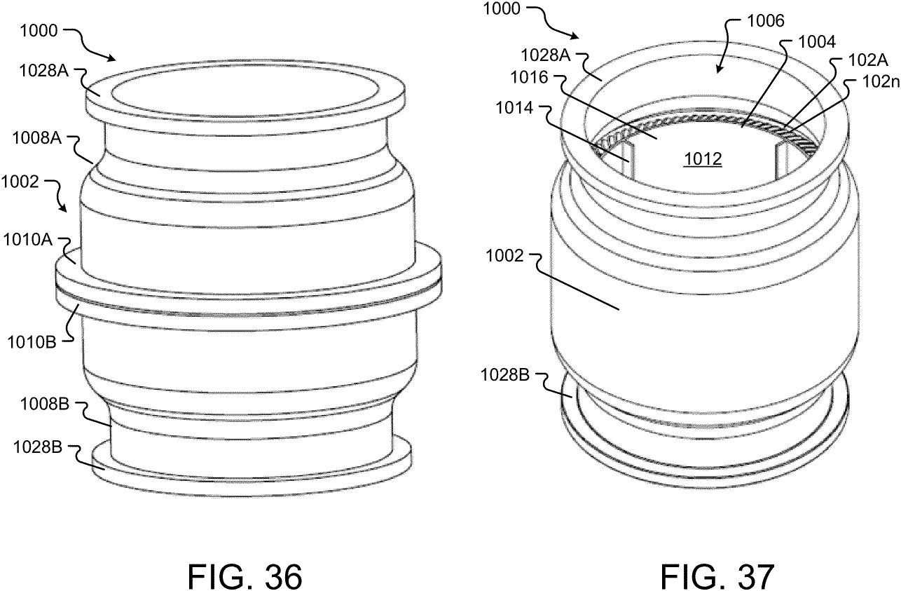

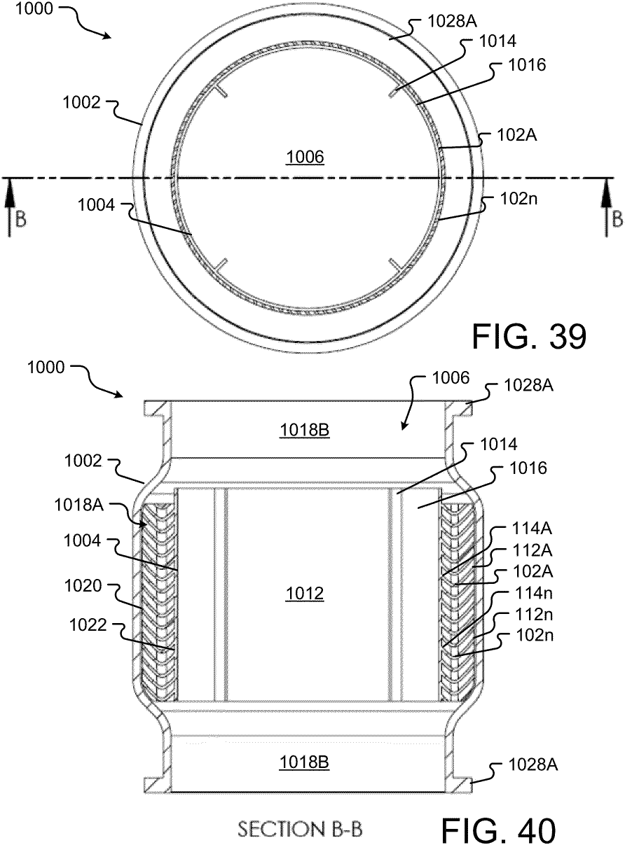

FIGS. 36-44 in general illustrate a bioreactor 1000 according to embodiments of the present disclosure. It is noted that portions of the bioreactor 1000 (e.g., housing 1002, or the like) in FIGS. 36-44 are example representations of an expanded cell culture vessel that is operable to replace a more-narrow (or smaller diameter) cylindrical vessel such as those manufactured and/or marketed by Eppendorf.

In addition, embodiments directed to the particle settler device 100, the bioreactor 600, the bioreactor 700, the bioreactor 800, the bioreactor 1100, the biosettler 1200, and/or the bioreactor 1300 may be applied to the bioreactor 1000 unless otherwise noted. In particular, the bioreactor 1000 is similar to other settler devices or bioreactors described herein and includes many of the same or similar features.

The bioreactor 1000 includes a housing 1002, a separation cylinder or inner cylinder 1004, and a cavity 1006 defined between the sidewalls of the housing 1002 and the inner cylinder 1004. For example, the housing 1002 may include portions similar to a cylinder, and the inner cylinder 1004 and the outer cylinder portions of the housing 1002 may be generally aligned or share a longitudinal axis.

In some embodiments, the housing 1002 is separable into multiple sections including, but not limited to, an upper housing section 1008A and a lower housing section 1008B. To assist in the joining of the housing sections 1008A / 1008B, the upper housing section 1008A may include an upper flange 1010A and the lower housing section 1008B may include a lower flange 1010B. It is noted the upper flange 1010A and/or the lower flange 1010B may be coupled (e.g., mechanically, or the like) via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the upper housing section 1008A and the lower housing section 1008B may be used with the bioreactor 1000. In addition, it is noted a washer or gasket may be positioned on (or within a groove in the rim of) the flange 1010A of the upper housing section 1008A and/or the rim or flange 1010B of the lower housing section 1008B during assembly of the bioreactor 1000. In other embodiments, the housing 1002 is of a single-piece construction.

In certain embodiments, the inner cylinder 1004 is hollow and defines a cavity 1012. For example, the cavity 1012 may be the main bioreactor space in which cells are suspended in a culture medium and growing as single cells. In some embodiments, the inner cylinder 1004 has fins or projections 1014 that extend inward from an interior surface 1016 of the inner cylinder 1004. For example, the fins or projections 1014 are operable to increase mixing and/or to avoid vortices when an impeller installed within the bioreactor 1000 is rotating. In other embodiments, the interior surface 1016 is smooth or substantially smooth.

In certain embodiments, the inner cylinder 1004 includes a different cross-section than the housing 1002. For example, the inner cylinder 1004 may include a constant outer diameter, while the housing 1002 includes a first, larger diameter proximate to the latitudinal center of the housing 1002, and at least a second, smaller diameter proximate to an end of the housing 1002. Due to the difference in cross-sections, the cavity 1006 includes a cavity portion 1018A and at least one cavity portion 1018B.

The cavity portion 1018A is configured to receive multiple sliders 102A-102n as described throughout the present disclosure. In other embodiments, the inner cylinder 1004 includes a similar cross-section to the housing 1002, such that only the cavity 1018A is defined between the sidewalls of the housing 1002 and the inner cylinder 1004 (with the sliders 102A-102n being installed in a portion or all of the cavity 1018A), without departing from the scope of the present disclosure.

The sliders 102A-102n are fabricated and fixed within the cavity portion 1018A via edges 112A-112n and 114A-114n at locations and/or spacings determined through experimentation or computational simulations. For example, the sliders 102A-102n may be 3D printed or injection molded or otherwise attached (e.g., glued, heat welded, or sonically welded) to an interior surface 1020 of the housing 1002 and/or an exterior surface 1022 of the inner cylinder 1004.

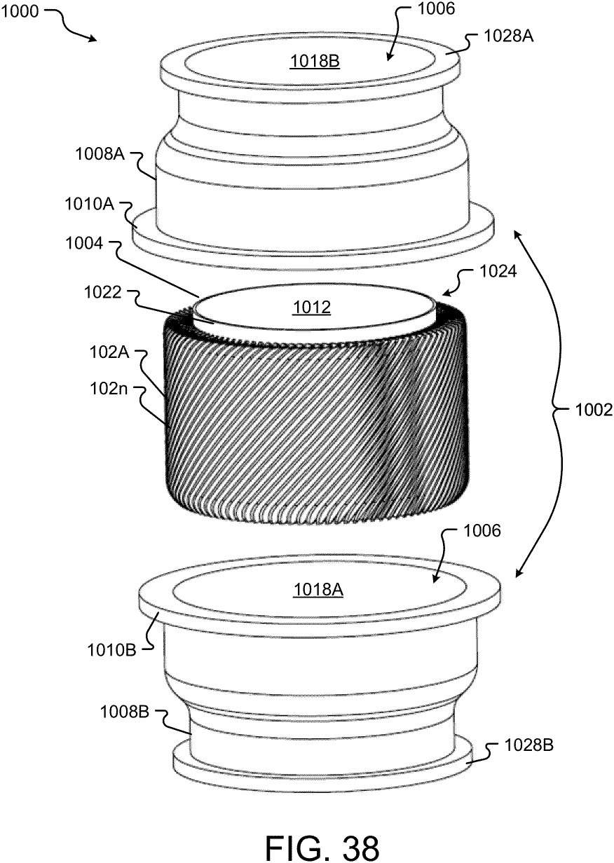

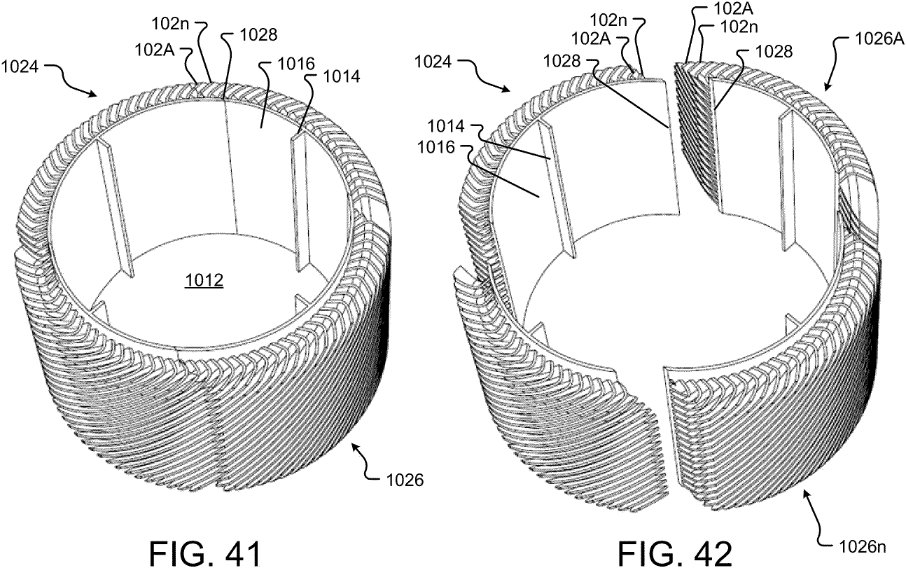

In certain embodiments, the sliders 102A-102n are fabricated and fixed to the inner cylinder 1004 in a single-piece construction (e.g., as illustrated in FIG. 38) as a settling assembly 1024. In other embodiments, as illustrated in FIGS. 41-44, the sliders 102A-102n are fabricated and fixed to the inner cylinder 1004 in two or more sections 1026A-1026n. Due to the downward-angled or inclined configuration of the sliders 102A-102n on the exterior surface 1022 of the inner cylinder 1004, wherein the sliders 102A-102n are angled with respect to the longitudinal axis of the housing 1002, dividing the settling assembly 1024 into the sections 1026 may result in sliders 102A-102n that intersect with a division cut to be split between adjacent sections 1026. For example, FIGS. 41-44 illustrate the sliders 102A-102n being oriented at a first angle with respect to the inner cylinder 1004, and a division cut 1028 that is oriented at a second different angle with respect to the inner cylinder 1004. Due to the difference in angles, the division cut 1028 separates both the inner cylinder 1004 and multiple of the sliders 102A-102n into different adjacent sections 1026. It is noted, however, that the sections 1026A-1026n are not limited to the embodiments depicted in FIGS. 41-44. For example, the division cut 1028 may be aligned with the sliders 102A-102n and/or may only cut through the inner cylinder 1004, such that the sliders 102A-102n are fully separated onto a particular section 1026 of the settling assembly 1024. In general, the sections 1026A-1026n may be fabricated as larger sections, smaller sections, and/or differently shaped sections for insertion into the housing 1002 than those depicted in FIGS. 41-44, without departing from the scope of the present disclosure.

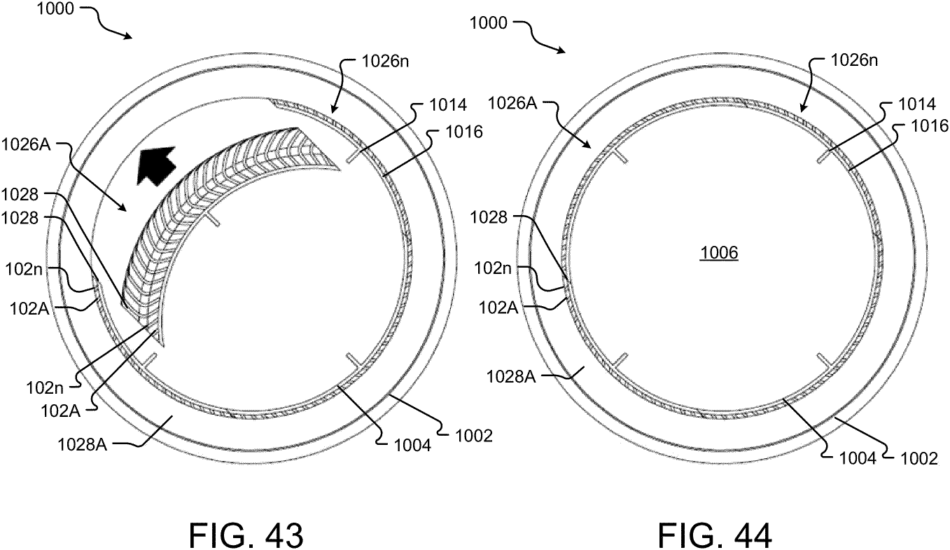

As illustrated in FIGS. 43 and 44, the sectioning of the settling assembly 1024 may facilitate an increased ease of installation of the settling assembly 1024 and/or may facilitate the retrofitting of existing bioreactors with the settling assembly 1024. For example, where the housing 1002 of the bioreactor 1000 is a commercially available product, the internals of the bioreactor 1000 may be retrofitted with the improved sliders 102A-102n via installation of the sections 1026 of the settling assembly 1024. By way of another example, the sections 1026 may be fit through an opening of the housing 1002 that is smaller in diameter than the diameter of the settling assembly 1024 when fully constructed within the housing 1002, without a need to dismantle or break the housing 1002.

In some embodiments, the sections 1026A-1026n may be substantially identical, to increase ease of manufacturing and potentially reduce manufacturing costs for the settling assembly 1024 (e.g., by fabricating the less-complex sections 1026 instead of fabricating the entire settling assembly 1024 at once). In other embodiments, the sections 1026 may be specially configured for a particular use when settling particles within the bioreactor 1000. In this regard, the sections 1026 may be customizable as necessary for specific uses. It is noted the fins or protrusions 1014 may be separated between the various sections 1026A-1026n, or may be specific to a particular section 1026.

In some embodiments, the bioreactor 1000 may include a lid or top closure and/or a bottom closure. For example, the top closure and/or the bottom closure may be planar or flat. By way of another example, the top closure and/or the bottom closure may be conical, hemispherical, semispherical or dome shaped, or other three-dimensional shape known in the art. It is noted herein the top closure and/or the bottom closure may be formed as part of the housing 1002. Discussion throughout the present disclosure related to various top closures is considered similarly applicable to the bioreactor 1000, where the bioreactor 1000 includes a lid or top closure and/or a bottom closure.

In some embodiments, the bioreactor 1000 includes a top flange 1028A and/or a bottom flange 1028B. In some embodiments, the bioreactor 1000 may include one or more inlet ports and one or more outlet ports within the housing 1002, the inner cylinder 1004, the top closure, and/or the bottom closure. For example, cell or particle contained fluid, multiple inlet fluids to be mixed, and/or overlay gas may be input via the one or more inlet ports, while concentrated cell or particle suspension and/or clarified supernatant (e.g., clarified culture fluid containing any metabolic waste products, such as ammonia and lactate, or gasses, along with any not-yet settled smaller dead cells and cell debris) may be output from the via the one or more outlet ports. Discussion throughout the present disclosure related to various inlet and outlet ports is considered similarly applicable to the bioreactor 1000, where the bioreactor 1000 includes inlet and outlet ports.

It is noted the top closure and/or the bottom closure may be coupled (e.g., mechanically, or the like) to the housing 1002 (e.g., to the top flange 1028A and/or the bottom flange 1028B, respectively) via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the lid or top closure or upper portion and/or the bottom closure or lower portion to the housing 1002 may be used with the bioreactor 1000. In addition, it is noted a washer or gasket may be positioned on the housing 1002 or within a groove in a rim or flange of the housing 1002 (not shown) between the housing 1002 and the top closure and/or the bottom closure during assembly of the bioreactor 1000.

In some embodiments, the cavity 1012 defined by the inner cylinder 1004 may be configured to receive an impeller with one or more sets of blades and related impeller components. Discussion throughout the present disclosure related to various impellers and related impeller components is considered similarly applicable to the bioreactor 1000, where the bioreactor 1000 includes the impeller and related impeller components.

Elements of the bioreactor 1000, such as the sliders 102 and the housing 1002 and inner cylinder 1004, may be fabricated of a single-use, disposable plastic. Alternatively, one or more of the elements of the bioreactor 1000 may be manufactured of a metal, such as a stainless-steel alloy, or glass. Depending on the fabrication material, at least a portion of the elements of the bioreactor 1000 may be partially or fully transparent or at least translucent to light of a predetermined range or ranges of wavelengths.

Surfaces within the bioreactor 1000, such as the surfaces 104 of the sliders 102, the interior surface 1020 of the housing 1002, and the exterior surface 1022 of the inner cylinders 1004, may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The bioreactor 1000 may be easily scaled to any desired size.

It is noted one or more of the ports of the bioreactor 1000 may be configured to couple (e.g., mechanically, fluidically, or the like) to external tubing lines. Such tubing line may be interconnected to any of the compact cell settler devices of the present disclosure. The line may have a diameter or otherwise be configured to interconnect to any port of embodiments of the present disclosure. Optionally, one or more of the ports of the bioreactor 1000 may be used for sampling bioreactor contents, for example to check cell viability, and continuous measurement of liquid pH and DO for inputs into a computer-controlled multi-gas mass flow controller. The line may optionally include at least one sensor positioned within a hollow interior. The sensors may be in contact with fluid and/or particles within the line. Optionally, the sensors may be arranged on an interior surface of the line, although other configurations are contemplated. The sensors may be operable to monitor one or more of pH, DO, glucose, temperature, and CO₂ (including dissolved or partial CO₂) in the line. Optionally, one or more of the sensors may comprise a fluorescent probe which emits light that varies based on a condition sensed by the probe. The light may be collected by a reader or meter. Optionally, the light may be collected by an optional fiber cable and transmitted to the meter. The meter is operable to report or display levels of at least one of pH, DO, glucose, temperature, and CO₂ sensed by the fluorescent probes. The tubing line may comprise a material that is transparent or at least translucent. Thus, light generated by a sensor may pass through the line. Alternatively, at least a portion of a line is transparent or at least translucent, similar to a window. Accordingly, light generated by a sensor may be transmitted through window portion and collected by the meter.

The housing 1002 and/or the inner cylinders 1004 may optionally include a fluid jacket (not illustrated). The fluid jacket can operate such that water or other fluids may be directed into the fluid jacket through one or more ports to maintain the housing 1002 and/or the inner cylinder 1004 and contents within the bioreactor 1000 within a desired temperature range. Optionally, a heater may be connected to one or more of the housing 1002 and/or the inner cylinders 1004 to adjust the temperature of liquid within the bioreactor 1000. For example, in one embodiment, a heat exchanger to heat or cool the inlet cell culture media can be connected to the ports.

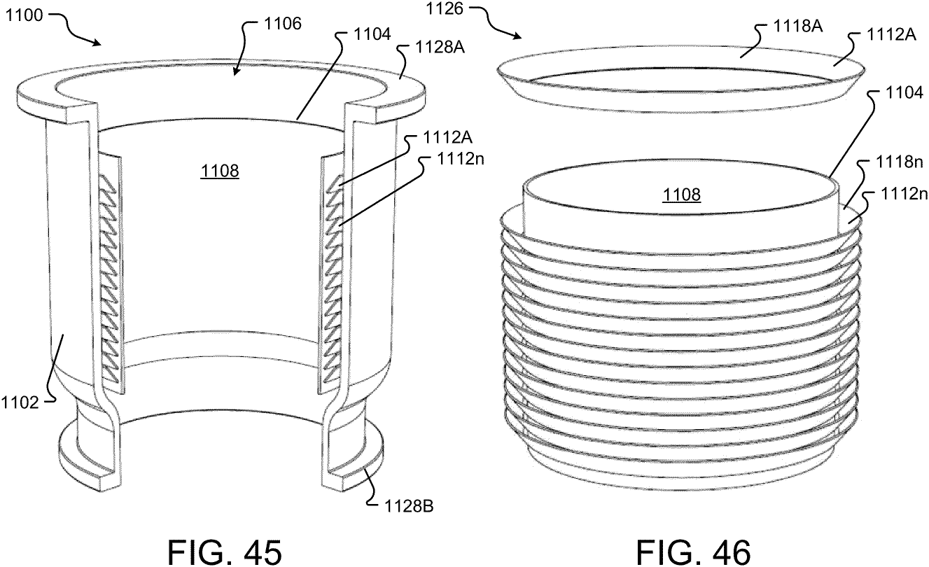

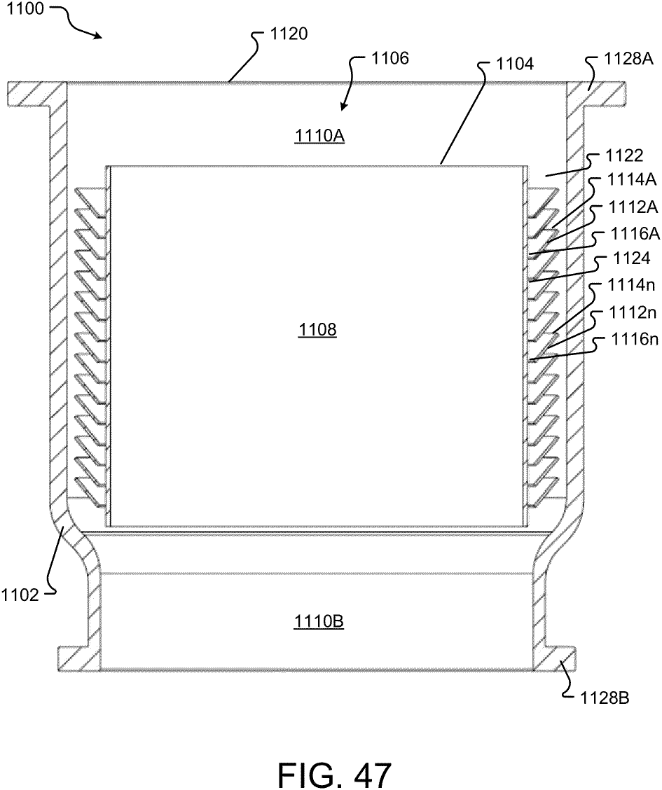

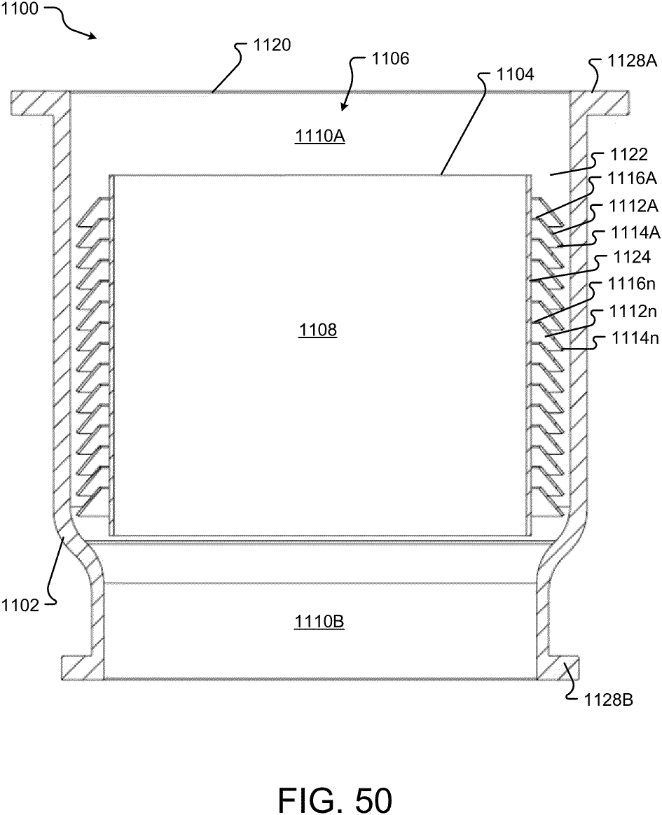

FIGS. 45-50 in general illustrate a bioreactor 1100 according to embodiments of the present disclosure. It is noted portions of the bioreactor 1100 (e.g., housing 1102) in FIGS. 45-50 are example representations of an expanded cell culture vessel that is operable to replace a more-narrow (or smaller diameter) cylindrical vessel such as those manufactured and/or marketed by Eppendorf. In FIGS. 36-44, the top and bottom openings of the housing 1002 are smaller in diameter than the cavity 1006 of the housing 1002, such that the settling assembly 1024 may be insertable in sections 1026A-1206n. In FIGS. 45-50, however, at least the top opening 1120 is closer (or substantially equal) in diameter to a diameter of a cavity 1106 within the housing 1102, such that a settling assembly 1126 may be insertable as a complete component or in sections.

In addition, embodiments directed to the particle settler device 100, the bioreactor 600, the bioreactor 700, the bioreactor 800, the bioreactor 1000, the biosettler 1200, and/or the bioreactor 1300 may be applied to the bioreactor 1100 unless otherwise noted. In particular, the bioreactor 1100 is similar to other settler devices or bioreactors described herein and includes many of the same or similar features.

The bioreactor 1100 includes a housing 1102, a separation cylinder or inner cylinder 1104, and a cavity 1106 defined between the sidewalls of the housing 1102 and the inner cylinder 1104. For example, the housing 1102 may include portions similar to a cylinder, and the inner cylinder 1104 and the outer cylinder portions of the housing 1102 may be generally aligned or share a longitudinal axis. In certain embodiments, the inner cylinder 1004 is hollow and defines a cavity 1108.

In certain embodiments, the inner cylinder 1104 includes a different cross-section than the housing 1102. For example, the inner cylinder 1104 may include a constant outer diameter, while the housing 1102 includes a first, larger diameter proximate to the latitudinal center of the housing 1102, and at least a second, smaller diameter proximate to an end of the housing 1102. Due to the difference in cross-sections, the cavity 1106 includes a cavity portion 1110A and at least one cavity portion 1110B.

The cavity portion 1110A is configured to receive multiple sliders 1112A-1112n, where “n” represents any number of sliders 1112. It is noted the sliders 1112A-1112n may include features similar to the sliders 102A-102n, the sliders 402A-402n, the sliders 1208A-1208n, and/or the sliders 1308A-1308n as described throughout the present disclosure. In other embodiments, the inner cylinder 1104 includes a similar cross-section to the housing 1102, such that only the cavity 1110A is defined between the sidewalls of the housing 1102 and the inner cylinder 1104 (with the sliders 1112A-1112n being installed in a portion or all of the cavity 1110A), without departing from the scope of the present disclosure.

As illustrated in FIGS. 45-50, the sliders 1112A-1112n are ring-shaped. The sliders 1112A-1112n include a body with a substantially circular cross-section, with opposite edges 1114A-1114n and edges 1116A-1116n, and with surfaces 1118A-1118n. To facilitate the settling of particles, in some embodiments either the edges 1114A-1114n or the edges 1116A-1116n may be spaced a predetermined distance from the interior surface 1120 of the housing 1102 and/or exterior surface 1124 of the inner cylinder 1104, respectively. This spacing, combined with the respective direction of surfaces 1118A-1118n of the housing 1102 and/or the inner cylinder 1104, facilitates the settling of particles.

In particular, in FIGS. 45-47 the sliders 1112A-1112n are downward-facing rings, with the edges 1114A-1114n closer to a top opening 1120 of the housing 1102 than the edges 1116A-1116n of the sliders 1112A-1112n such that the surfaces 1118A-1118n are inclined downward toward a lower end of the housing 1102, being angled with respect to the longitudinal axis of the housing 1102. The surfaces 1118A-1118n may be inclined or sloped downward in a direction toward the inner cylinder 1104, such that settling particles may be directed toward the inner cylinder 1104. In the embodiments depicted in FIGS. 45-47, the sliders 1112A-1112n may be considered concave.

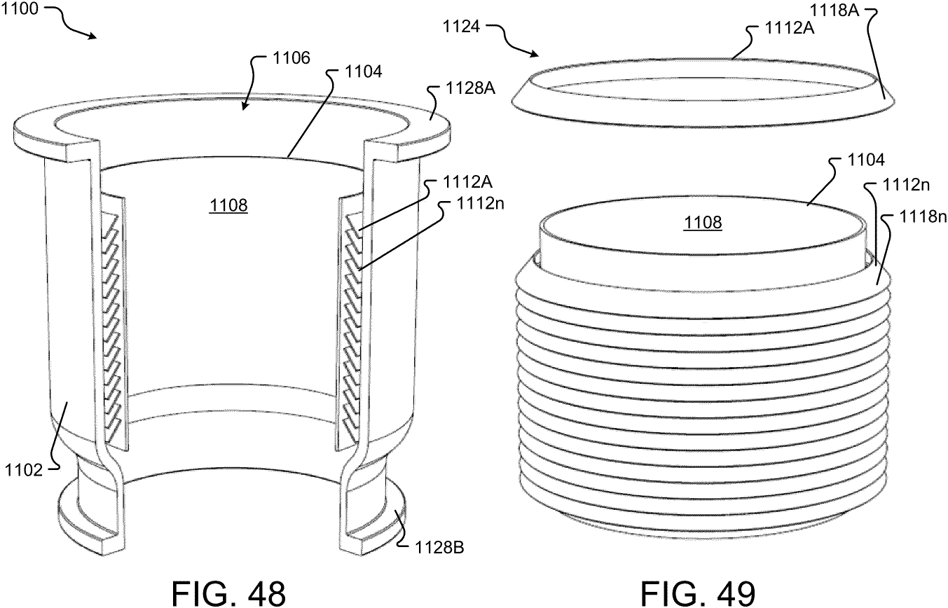

In addition, in FIGS. 48-50 the sliders 1112A-1112n are upward-facing rings, with the edges 1116A-1116n closer to a top opening of the housing 1102 than the edges 1114A-1114n of the sliders 1112A-1112n such that the surfaces 1118A-1118n are inclined upward toward an upper end of the housing 1102, being angled with respect to the longitudinal axis of the housing 1202. The surfaces 1118A-1118n may be inclined or sloped upward in a direction toward the inner cylinder 1104, such that settling particles may be directed away from the inner cylinder 1104. In the embodiments depicted in FIGS. 48-50, the sliders 1112A-1112n may be considered convex.

It is noted that the settlers 1112A-1112n with the inclined surfaces 1118A-1118n may be understood as representing sections of conical sliders, as compared to sliders 102A-102n and 402A-402n that are more planar in shape (e.g., either with flat surfaces or arcuate surfaces, respectively). In addition, it is contemplated that the settlers 1112A-1112n may be of increased use when making thin plastic films in single use disposable bioreactors.

The sliders 1112A-1112n are fabricated and fixed within the cavity portion 1010A via edges 1114A-1114n and 1116A-1116n at locations and/or spacings determined through experimentation or computational simulations. For example, the sliders 1112A-1112n may be 3D printed or injection molded or otherwise attached (e.g., glued, heat welded, or sonically welded) to an interior surface 1120 of the housing 1102 and/or an exterior surface 1124 of the inner cylinder 1104. For example, the sliders 1112A-1112n may be welded or otherwise attached at three or more intermittent locations to the exterior surface 1124 of the inner cylinder 1104. It is noted the sliders 1112A-1112n may include an inner diameter that is at least the outer diameter of the inner cylinder 1104, to facilitate the positioning of the sliders 1112A-1112n around the inner cylinder 1104. It is noted the sliders 1112A-1112n and the inner cylinder 1104 may be considered a settling assembly 1126, for purposes of the present disclosure.

The sliders 1112A-1112n being ring-shaped may facilitate an increased ease of installation of the settling assembly 1126 and/or may facilitate the retrofitting of existing bioreactors with the settling assembly 1126. For example, where the housing 1102 of the bioreactor 1100 is a commercially available product, the internals of the bioreactor 1100 may be retrofitted with the improved sliders 1112A-1112n via installation of each individual slider 1112 (or groups of sliders 1112) about an installed inner cylinder 1104. The sliders 1112 may be substantially identical, to increase ease of manufacturing and potentially reduce manufacturing costs for the settling assembly 1126 (e.g., by fabricating the less-complex sliders 1112 and inner cylinder 1104 instead of fabricating the entire settling assembly 1126 at once).

In some embodiments, the housing 1102 is separated into multiple sections including, but not limited to, an upper housing section and a lower housing section. In some embodiments, the inner cylinder 1104 has fins or projections that extend inward from an interior surface of the inner cylinder 1104. In other embodiments, the interior surface is smooth or substantially smooth.

In some embodiments, the bioreactor 1100 may include a lid or top closure and a bottom closure. For example, the top closure and/or the bottom closure may be planar or flat. By way of another example, the top closure and/or the bottom closure may be conical, hemispherical, semispherical or dome shaped, or other three-dimensional shape known in the art. It is noted herein the top closure and/or the bottom closure may be formed as part of the housing 1102. Discussion throughout the present disclosure related to various top closures is considered similarly applicable to the bioreactor 1100, where the bioreactor 1100 includes a lid or top closure and/or a bottom closure.

In some embodiments, the bioreactor 1100 includes a top flange 1128A and/or a bottom flange 1128B. In some embodiments, the bioreactor 1100 may include one or more inlet ports and one or more outlet ports within the housing 1102, the inner cylinder 1104, the top closure, and/or the bottom closure. For example, cell or particle contained fluid, multiple inlet fluids to be mixed, and/or overlay gas may be input via the one or more inlet ports, while concentrated cell or particle suspension and/or clarified supernatant (e.g., clarified culture fluid containing any metabolic waste products, such as ammonia and lactate, or gasses, along with any not-yet settled smaller dead cells and cell debris) may be output from the via the one or more outlet ports. Discussion throughout the present disclosure related to various inlet and outlet ports is considered similarly applicable to the biosettler 1200, where the biosettler 1200 includes inlet and outlet ports.

It is noted the top closure and/or the bottom closure may be coupled (e.g., mechanically, or the like) to the housing 1102 (e.g., to the top flange 1128A and/or the bottom flange 1128B, respectively) via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the lid or top closure or upper portion and/or the bottom closure or lower portion to the housing 1102 may be used with the bioreactor 1100. In addition, it is noted a washer or gasket may be positioned on the housing 1102 or within a groove in a rim or flange of the housing 1102 (not shown) between the housing 1102 and the top closure and/or the bottom closure during assembly of the bioreactor 1100.

In some embodiments, the cavity 1012 defined by the inner cylinder 1104 may be configured to receive an impeller with one or more sets of blades and related impeller components. Discussion throughout the present disclosure related to various impellers and related impeller components is considered similarly applicable to the bioreactor 1100, where the bioreactor 1100 includes the impeller and related impeller components.

Elements of the bioreactor 1100, such as the sliders 1112 and the housing 1102 and inner cylinder 1104, may be fabricated of a single-use, disposable plastic. Alternatively, one or more of the elements of the bioreactor 1100 may be manufactured of a metal, such as a stainless-steel alloy, or glass. Depending on the fabrication material, at least a portion of the elements of the bioreactor 1100 may be partially or fully transparent or at least translucent to light of a predetermined range or ranges of wavelengths.

Surfaces within the bioreactor 1100, such as upper surfaces 1500A-1500n of the sliders 1112A-1112n, the interior surface 1120 of the housing 1102, and the exterior surface 1124 of the inner cylinders 1104, may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The bioreactor 1100 may be easily scaled to any desired size.

It is noted one or more of the ports of the bioreactor 1100 may be configured to couple (e.g., mechanically, fluidically, or the like) to external tubing lines. Such tubing line may be interconnected to any of the compact cell settler devices of the present disclosure. The line may have a diameter or otherwise be configured to interconnect to any port of embodiments of the present disclosure. Optionally, one or more of the ports of the bioreactor 1100 may be used for sampling bioreactor contents, for example to check cell viability, and continuous measurement of liquid pH and DO for inputs into a computer-controlled multi-gas mass flow controller. The line may optionally include at least one sensor positioned within a hollow interior. The sensors may be in contact with fluid and/or particles within the line. Optionally, the sensors may be arranged on an interior surface of the line, although other configurations are contemplated. The sensors may be operable to monitor one or more of pH, DO, glucose, temperature, and CO₂ (including dissolved or partial CO₂) in the line. Optionally, one or more of the sensors may comprise a fluorescent probe which emits light that varies based on a condition sensed by the probe. The light may be collected by a reader or meter. Optionally, the light may be collected by an optional fiber cable and transmitted to the meter. The meter is operable to report or display levels of at least one of pH, DO, glucose, temperature, and CO₂ sensed by the fluorescent probes. The tubing line may comprise a material that is transparent or at least translucent. Thus, light generated by a sensor may pass through the line. Alternatively, at least a portion of a line is transparent or at least translucent, similar to a window. Accordingly, light generated by a sensor may be transmitted through window portion and collected by the meter.

The housing 1102 and/or the inner cylinders 1104 may optionally include a fluid jacket (not illustrated). The fluid jacket can operate such that water or other fluids may be directed into the fluid jacket through one or more ports to maintain the housing 1102 and/or the inner cylinder 1104 and contents within the bioreactor 1100 within a desired temperature range. Optionally, a heater may be connected to one or more of the housing 1102 and/or the inner cylinders 1104 to adjust the temperature of liquid within the bioreactor 1100. For example, in one embodiment, a heat exchanger to heat or cool the inlet cell culture media can be connected to the ports.

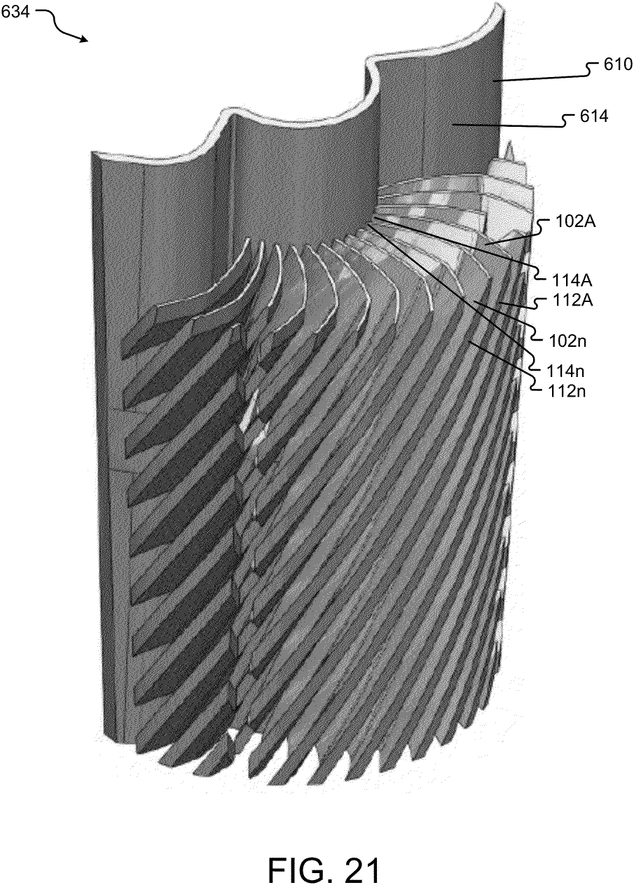

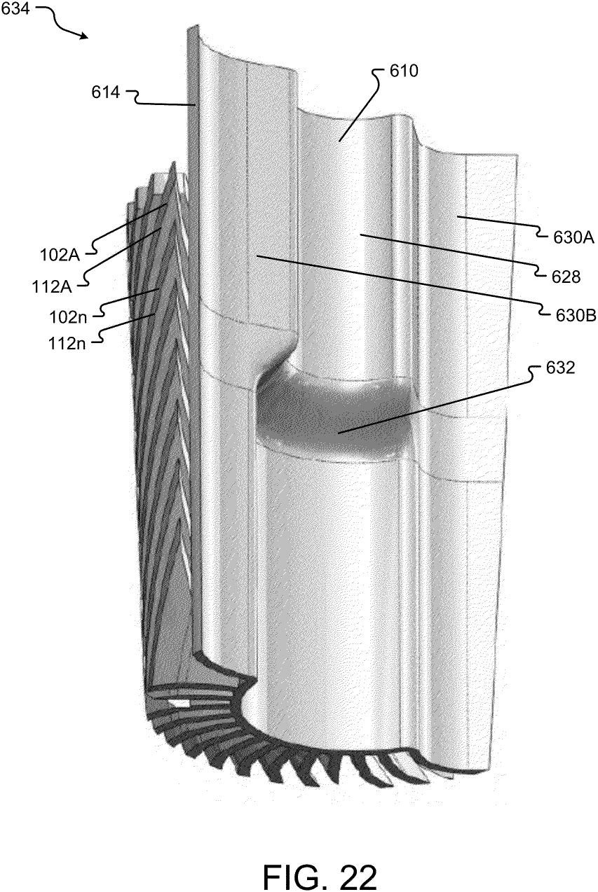

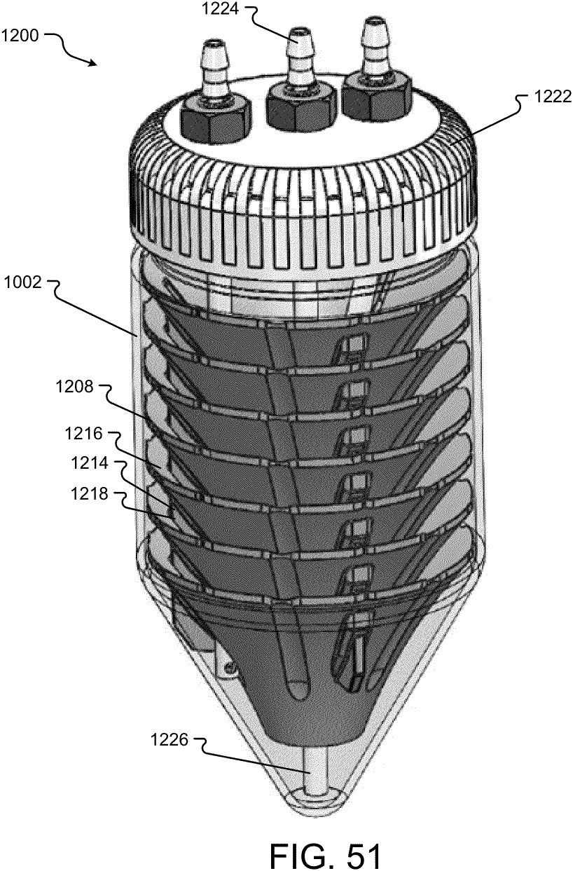

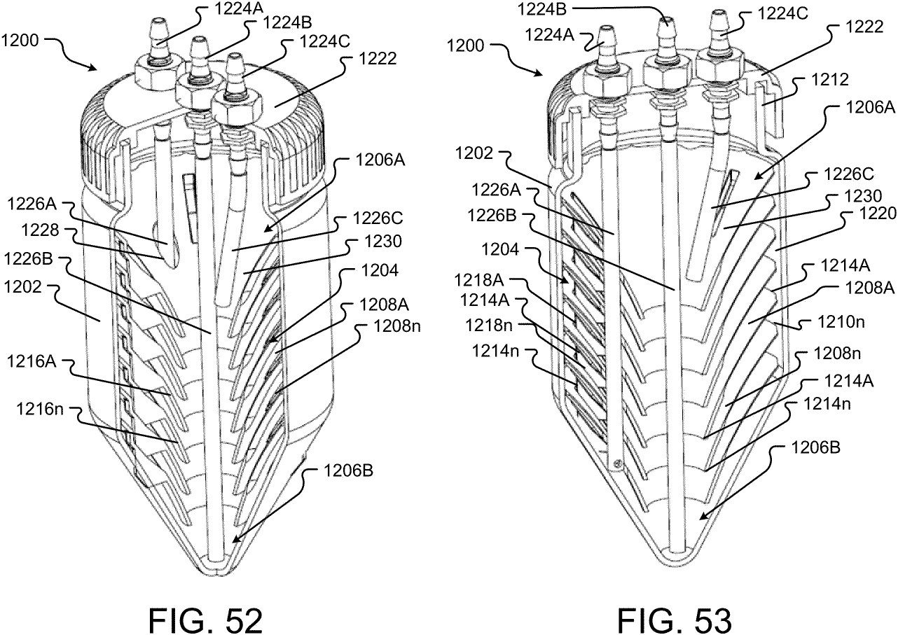

FIGS. 51-53 in general illustrate a settling device or biosettler 1200 according to embodiments of the present disclosure. It is noted portions of the biosettler 1200 in FIGS. 51-53 may be attached to any suspension bioreactor (e.g., the bioreactor 600 depicted in FIGS. 15-22, or the like) as described throughout the present disclosure. Referring to bioreactor 600, in one non-limiting example the biosettler 1200 may be used in place of the settling assembly 634 of the bioreactor 600, as depicted in at least FIGS. 21 and 22.

In addition, embodiments directed to the particle settler device 100, the bioreactor 600, the bioreactor 700, the bioreactor 800, the bioreactor 1000, the bioreactor 1100, and/or the bioreactor 1300 may be applied to the biosettler 1200 unless otherwise noted. In particular, the biosettler 1200 is similar to other settler devices or bioreactors described herein and includes many of the same or similar features.

The biosettler 1200 includes a housing 1202 and a cavity 1204 defined within the housing 1202. For example, the housing 1202 may include portions similar to a cylinder. It is noted components of the biosettler 1200 (e.g., including the housing 1202) may be illustrated as transparent for clarity in FIG. 51, but that one having skill in the art would understand this not to be limiting on the present disclosure.

In certain embodiments, the housing 1202 includes a different cross-section along its length. For example, the housing 1202 may include a first, larger diameter proximate to the latitudinal center of the housing 1202, and at least a second, smaller diameter proximate to an end of the housing 1202. For example, the portion of the housing 1202 having the at least a second, smaller diameter proximate to the end of the housing 1202 may be conical or cone-shaped, tapering from the diameter of the first section of the housing 1202 to the end of the housing 1202. Due to the difference in cross-sections, the cavity 1204 includes a cavity portion 1206A and at least one cavity portion 1206B.

The cavity portion 1206A is configured to receive multiple sliders 1208A-1208n, where “n” represents any number of sliders 1208. It is noted the sliders 1208A-1208n may include features similar to the sliders 102A-102n, the sliders 402A-402n, the sliders 1112A-1112n, and/or the sliders 108A-1208n as described throughout the present disclosure. In other embodiments, the housing 1202 includes a substantially constant cross-section along its length, such that only the cavity 1206A is defined within the housing 1202, without departing from the scope of the present disclosure.

As illustrated in FIGS. 51-53, the sliders 1208A-1208n are conical or cone-shaped. In particular, in FIGS. 51-53 the sliders 1208A-1208n are downward-facing cones, with upper openings 1210A-1210n having a first diameter positioned closer to a top opening 1212 of the housing 1202 than lower openings 1214A-1214n having a second smaller diameter. It is noted, however, the sliders 1208A-1208n may be upward-facing cones, with the upper openings 1210A-1210n having a first diameter and the lower openings 1212A-1212n having a second larger diameter, without departing from the scope of the present disclosure.

In some embodiments, the sliders 1208A-1208n has fins or projections 1214A-1214n extending from contoured walls 1216A-1216n. For example, the fins or projections 1214 of a first slider 1208 may make contact with the contoured wall 1216 of an adjacent slider 1208. In some embodiments, the sliders 1208A-1208n has fins or projections 1214A-1214n and opposite recesses or apertures 1218A-1218n within the contoured wall 1216A-1216n. For example, the fins or projections 1214 of a first slider 1208 may be at least partially insertable within the corresponding recess or aperture 1218 of an adjacent slider 1208. By way of another example, the fins or projections 1214 may be disposed against an interior surface 1220 of the housing 1202. In these embodiments, the fins or projections 1214A-1214n serve to position the sliders 1208 within the housing 1202 and space adjacent sliders 1208 with an offset position. In other embodiments, the sliders 1208A-1208n have smooth or substantially smooth contoured surfaces, without departing from the scope of the present disclosure.

In certain embodiments, the sliders 1208A-1208n may be fabricated and fixed within the cavity portion 1204 at locations and/or spacings determined through experimentation or computational simulations. For example, the sliders 1208A-1208n may be 3D printed or injection molded or otherwise attached (e.g., glued, heat welded, or sonically welded) to the interior surface 1220 of the housing 1202. It is noted, however, that the sliders 1208A-1208n may be positioned within the housing 1202 without the need for additional fixation, without departing from the scope of the present disclosure.

The sliders 1208A-1208n being ring-shaped may facilitate an increased ease of installation and/or may facilitate the retrofitting of existing bioreactors. For example, where the housing 1202 of the biosettler 1200 is a commercially available product, the internals of the biosettler 1200 may be retrofitted with the improved sliders 1208A-1208n via installation of each individual sliders 1208 (or groups of sliders 1208). The sliders 1208 may be substantially identical, to increase ease of manufacturing and potentially reduce manufacturing costs.

The biosettler 1200 includes a lid or top closure 1222. For example, the top closure 1222 may be planar or flat, conical, hemispherical, semispherical or dome shaped, or other three-dimensional shape known in the art. It is noted herein the top closure 1222 may be separate from or formed as part of the housing 1202. In addition, it is noted the portion of the housing 1202 having the at least a second, smaller diameter may be formed as part of the housing 1202, or may be a separate bottom closure. Discussion throughout the present disclosure related to various top closures is considered similarly applicable to the biosettler 1200.

The biosettler 1200 includes one or more ports 1224 (e.g., inlet ports and/or outlet ports) within the housing 1202, the inner cylinder 1204, and/or the top closure 1214. For example, cell or particle contained fluid, multiple inlet fluids to be mixed, and/or overlay gas may be input via the one or more inlet ports, while concentrated cell or particle suspension and/or clarified supernatant (e.g., clarified culture fluid containing any metabolic waste products, such as ammonia and lactate, or gasses, along with any not-yet settled smaller dead cells and cell debris) may be output from the via the one or more outlet ports. Discussion throughout the present disclosure related to various inlet and outlet ports is considered similarly applicable to the biosettler 1200.

In one non-limiting example, at least one port 1224 may be coupled to internal tubing 1226 within the biosettler 1200. For instance, a port 1224A may be coupled to internal tubing 1226A routed through an aperture 1228 in a contoured surface 1230 of the sliders 1208A-1208n. In addition, a port 1224B may be coupled to internal tubing 1226B passed through the lower openings 1214A-1214n of the sliders 1208A-1208n. Further, a port 1226C may be coupled to internal tubing 1226C generally disposed within the housing 1202 and/or resting against the contoured surface 1230 of the sliders 1208A-1208n. It is noted that the surfaces 1230 may be considered inclined or sloped, being angled with respect to a longitudinal axis of the housing 1202.

It is noted the top closure 1222 may be coupled (e.g., mechanically, or the like) to the housing 1202 via a removable or releasable interlocking device (e.g., a flange and catch or hook assembly, a tab and groove assembly, mating threads, a fastener, or the like) or via non-removable means (e.g., an adhesive, a glue, a weld (e.g., heat weld or sonic weld, or the like). As will be appreciated by one of skill in the art, any suitable means of joining the lid or top closure or upper portion 1222 to the housing 1202 may be used with the biosettler 1200. In addition, it is noted a washer or gasket may be positioned on the housing 1202 or within a groove in a rim or flange of the housing 1202 (not shown) between the housing 1202 and the top closure 1222 during assembly of the biosettler 1200.

Elements of the biosettler 1200, such as the sliders 1208 and the housing 1202, may be fabricated of a single-use, disposable plastic. Alternatively, one or more of the elements of the biosettler 1200 may be manufactured of a metal, such as a stainless-steel alloy, or glass. Depending on the fabrication material, at least a portion of the elements of the biosettler 1200 may be partially or fully transparent or at least translucent to light of a predetermined range or ranges of wavelengths.

Surfaces within the biosettler 1200, such as the surfaces 1230 of the sliders 1208 and an interior surface 1220 of the housing 1202, may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The biosettler 1200 may be easily scaled to any desired size.

It is noted one or more of the ports of the biosettler 1200 may be configured to couple (e.g., mechanically, fluidically, or the like) to external tubing lines. Such tubing line may be interconnected to any of the compact cell settler devices of the present disclosure. The line may have a diameter or otherwise be configured to interconnect to any port of embodiments of the present disclosure. Optionally, one or more of the ports of the biosettler 1200 may be used for sampling bioreactor contents, for example to check cell viability.

The housing 1202 may optionally include a fluid jacket (not illustrated). The fluid jacket can operate such that water or other fluids may be directed into the fluid jacket through one or more ports to maintain the housing 1202 and contents within the biosettler 1200 within a desired temperature range. Optionally, a heater may be connected to the housing 1202 to adjust the temperature of liquid within the biosettler 1200. For example, in one embodiment, a heat exchanger to heat or cool the inlet cell culture media can be connected to the ports.

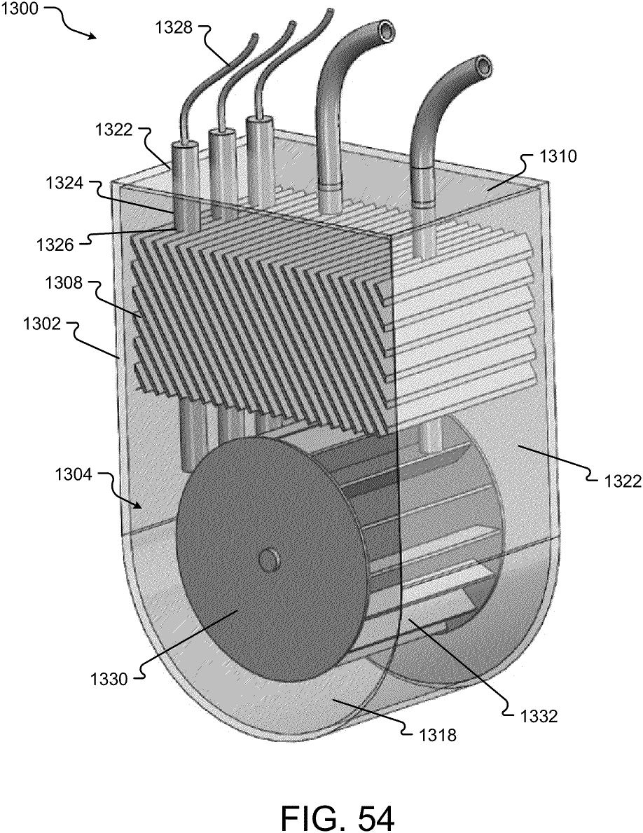

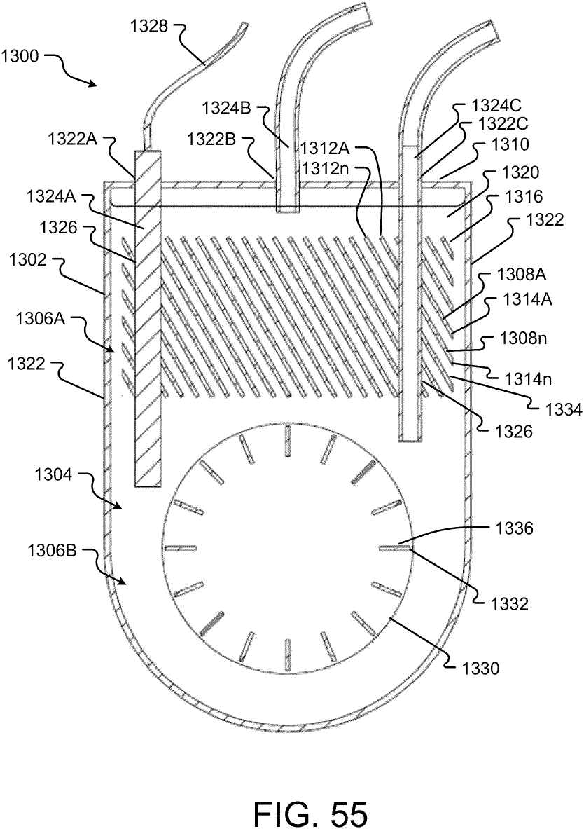

FIGS. 54-55 in general illustrate a bioreactor 1300 according to embodiments of the present disclosure. It is noted portions of the bioreactor 1300 (e.g., housing 1302) in FIGS. 54-55 are example representations of components of a Vertical-Wheel® bioreactor manufactured and/or marketed by PBS Biotech®. The representations are provided as an example that the sliders 1308 of the bioreactor 1300 are configurable for use with components of the Vertical-Wheel® bioreactor manufactured and/or marketed by PBS Biotech®.

In addition, embodiments directed to the particle settler device 100, the bioreactor 600, the bioreactor 700, the bioreactor 800, the bioreactor 1000, the bioreactor 1100, and/or the biosettler 1200 may be applied to the bioreactor 1300 unless otherwise noted. In particular, the bioreactor 1300 is similar to other settler devices or bioreactors described herein and includes many of the same or similar features.

The bioreactor 1300 includes a housing 1302 and a cavity 1304 defined within the housing 1302. It is noted components of the bioreactor 1300 (e.g., including the housing 1302) may be illustrated as transparent for clarity in FIG. 54, but that one having skill in the art would understand this not to be limiting on the present disclosure.

In certain embodiments, the housing 1302 includes a different cross-section along its height. For example, the housing 1302 may include a first portion with a first, larger width proximate to the latitudinal center of the housing 1302, and a second portion with at least a second, smaller diameter proximate to an end of the housing 1302. For example, the second portion of the housing 1302 may be hemispherical, having a diameter substantially equal to the width of the first section of the housing 1302 and tracing an arc to the end of the housing 1302 based on a corresponding radius, such that the total width of the second portion of the housing 1302 proximate to the end continually decreases along the height of the second portion. Due to the difference in cross-sections, the cavity 1304 includes a cavity portion 1306A and at least one cavity portion 1306B.

The cavity portion 1306A is configured to receive multiple sliders 1308A-1308n, where “n” represents any number of sliders 1308. It is noted the sliders 1308A-1308n may include features similar to the sliders 102A-102n, the sliders 402A-402n, the sliders 1112A-1112n, and/or the sliders 1208A-1208n as described throughout the present disclosure. In other embodiments, the housing 1302 includes a substantially constant cross-section along its length, such that only the cavity 1306A is defined within the housing 1302, without departing from the scope of the present disclosure.

As illustrated in FIGS. 54-55, the sliders 1308A-1308n are substantially planar and inclined or sloped with respect to a top surface 1310 of the housing 1302. In particular, in FIGS. 54-55 the sliders 1308A-1308n are inclined downward with respect to the top surface 1310, with an upper edge 1312A-1312n positioned closer to the top surface 1310 of the housing 1302 than a lower edge 1314A-1314n. It is noted, however, the sliders 102A-102n may be used in the bioreactor 1300 instead of the sliders 1308A-1308n, without departing from the scope of the present disclosure.

In certain embodiments, the sliders 1308A-1308n may be fabricated and fixed within the cavity portion 1304 at locations and/or spacings determined through experimentation or computational simulations. For example, the sliders 1308A-1308n may be 3D printed or injection molded or otherwise attached (e.g., glued, heat welded, or sonically welded) to an interior surface 1316 of a front surface 1318 or a rear surface 1320 of the housing 1302. It is noted, however, that the sliders 1308A-1308n may be positioned within the housing 1302 without the need for additional fixation, without departing from the scope of the present disclosure.

Although embodiments of the present disclosure illustrate the top surface 1310 being formed with or integrated with the front surface 1316, the rear surface 1318, and/or side surfaces 1320 of the housing 1302, it is noted herein the top surface 1310 may be a lid or top closure separate from and coupled to the housing 1302 without departing from the scope of the present disclosure. For example, the top closure may be planar or flat, conical, hemispherical, semispherical or dome shaped, or other three-dimensional shape known in the art. It is noted herein the second portion of the housing 1302 having the at least a second, smaller diameter may also be formed as part of the housing 1302, or may be a separate bottom closure. Discussion throughout the present disclosure related to various top closures is considered similarly applicable to the bioreactor 1300, where the top surface 1310 is a lid or top closure.

The bioreactor 1300 includes one or more ports 1322 (e.g., inlet ports and/or outlet ports) within the housing 1202 and/or the top surface 1310. The one or more ports 1322 may be apertures or may be physical components installed within the apertures. For example, cell or particle contained fluid, multiple inlet fluids to be mixed, and/or overlay gas may be input via the one or more inlet ports, while concentrated cell or particle suspension and/or clarified supernatant (e.g., clarified culture fluid containing any metabolic waste products, such as ammonia and lactate, or gasses, along with any not-yet settled smaller dead cells and cell debris) may be output from the via the one or more outlet ports. Discussion throughout the present disclosure related to various inlet and outlet ports is considered similarly applicable to the bioreactor 1300.

In one non-limiting example, at least one port 1322 may be coupled to internal tubing 1324 within the bioreactor 1300. For instance, a port 1322A may be coupled to internal tubing 1324A routed through apertures 1326 in the sliders 1308A-1308n and coupled (e.g., mechanically, fluidically, or the like) to additional external tubing lines 1328. In addition, a port 1322B may be coupled to internal tubing 1324B disposed above the sliders 1308A-1308n, where the internal tubing 1324B also extends externally from the housing 1302. Further, a port 1324C may be coupled to internal tubing 1324C routed through apertures 1326 in the sliders 1308A-1308n, where the internal tubing 1324C also extends externally from the housing 1302.

It is noted the external tubing lines 1328 may be interconnected to any of the compact cell settler devices of the present disclosure. The line may have a diameter or otherwise be configured to interconnect to any port of embodiments of the present disclosure. Optionally, one or more of the ports of the bioreactor 1300 may be used for sampling bioreactor contents, for example to check cell viability, and continuous measurement of liquid pH and DO for inputs into a computer-controlled multi-gas mass flow controller. The line may optionally include at least one sensor positioned within a hollow interior. The sensors may be in contact with fluid and/or particles within the line. Optionally, the sensors may be arranged on an interior surface of the line, although other configurations are contemplated. The sensors may be operable to monitor one or more of pH, DO, glucose, temperature, and CO₂ (including dissolved or partial CO₂) in the line. Optionally, one or more of the sensors may comprise a fluorescent probe which emits light that varies based on a condition sensed by the probe. The light may be collected by a reader or meter. Optionally, the light may be collected by an optional fiber cable and transmitted to the meter. The meter is operable to report or display levels of at least one of pH, DO, glucose, temperature, and CO₂ sensed by the fluorescent probes. The tubing line may comprise a material that is transparent or at least translucent. Thus, light generated by a sensor may pass through the line. Alternatively, at least a portion of a line is transparent or at least translucent, similar to a window. Accordingly, light generated by a sensor may be transmitted through window portion and collected by the meter.

In at least a portion of the cavity 1304, the bioreactor 1302 may house an impeller 1330 having one or more fins or blades 1332. In one non-limiting example, the impeller 1330 may be positioned within the cavity 1304 below the sliders 1308A-1308n, similar to a water wheel that corresponds to the arcuate second section of the housing 1302. The impeller 1330 may cause settling particles such as microcarrier particles or semi-solid single cells or cellular aggregates or organoids to recirculate. Alternatively, the impeller 1330 may agitate the fluid and cause the settling particles to unseat in the bottom of the arcuate second section of the housing 1302 to be delivered to an outlet port 1322 (e.g., where the outlet port 1322 is positioned between the bottom edge of the sliders or 1308A-1308n and the impeller 1330). It is noted the bioreactor 1300 may include other outlets for carrying the clarified supernatant (e.g., clarified culture fluid containing any metabolic waste products, such as ammonia and lactate, or gasses, along with any not-yet settled smaller dead cells and cell debris) out of the housing 1302. In certain embodiments, the impeller 1330 may be rotated by a power transmission device (not illustrated). For example, the power transmission device may be an electric motor, a turbine, an induction device, or another device capable of being driven by electricity, fluid, or magnetic forces. In certain embodiments, the impeller 1330 may be propelled by the settling particles falling from the sliders 1302A-1302n. Discussion throughout the present disclosure related to various impellers and related impeller components is considered similarly applicable to the bioreactor 1000.

Elements of the bioreactor 1300, such as the sliders 1308, the housing 1302, or the impeller 1330 with blades 1332 may be fabricated of a single-use, disposable plastic. Alternatively, one or more of the elements of the bioreactor 1300 may be manufactured of a metal, such as a stainless-steel alloy, or glass. Depending on the fabrication material, at least a portion of the elements of the bioreactor 1300 may be partially or fully transparent or at least translucent to light of a predetermined range or ranges of wavelengths.

Surfaces within the bioreactor 1300, such as surfaces 1334 of the sliders 1308, an interior surface 1310 of the housing 1302, and/or surfaces 1336 of the blades 1332 of the impeller 1330 may be completely or partially coated with one or more of a non-stick plastic, Teflon®, silicone and similar materials known to those of skill in the art. Additionally, or alternatively, the surfaces (especially when formed of stainless steel) may be electropolished to provide a smooth surface. The bioreactor 1300 may be easily scaled to any desired size.

The housing 1302 may optionally include a fluid jacket (not illustrated). The fluid jacket can operate such that water or other fluids may be directed into the fluid jacket through one or more ports to maintain the housing 1302 and contents within the bioreactor 1300 within a desired temperature range. Optionally, a heater may be connected to the housing 1302 to adjust the temperature of liquid within the bioreactor 1300. For example, in one embodiment, a heat exchanger to heat or cool the inlet cell culture media can be connected to the ports.

Exemplary methods of using the settler devices of this disclosure are now described. A particle containing liquid (including, for example, cell culture liquid, wastewater or reaction fluid containing solid catalyst particles, etc.) is introduced into a settler device of this disclosure though a port. Approximately 50% - 99% of the entering liquid (typically about 90%) is removed through a port at the bottom of the settler device, while the remaining 1% - 50% (typically about 10%) of the liquid is removed through a port at the top of the settler devices. A pump (e.g., a peristaltic pump, or the like) may be used to aspirate or suck liquid out of the top port, while the concentrated liquid exiting the bottom may exit the bottom outlet of the housing due to gravity, without the need for a pump. Alternately, the liquid containing the settled cells or particles, may be pumped out from a bottom port of the settler devices at about 50% - 99% of entering liquid flow rate, and the remaining clarified liquid (1% - 50%) may exit via a top port. Optionally, fluid exiting the port may be pumped out into a harvest line.

Most of the entering cells (or particles) are pushed against the walls of the inner structure (e.g., the inner cylinders 202, 704, 804, the separation plate 610, or the like) through centrifugal forces upon entry, settle down the inner structure through a gentle vortex motion initially, getting faster as the liquid and particles/cells go down and exit via a bottom port. Cells or particles which have not settled will move up through the inclined sliders 102, 404. As the liquid moves slowly up through the inclined sliders 102, 404, bigger particles (e.g., live cells) will settle on the surfaces of the inclined sliders 102, 404 and either slide down the inclined sliders 102, 404 or fall down any small spacing provided between the inclined sliders 102, 404 and the surfaces of the housing 102 or the inner structures. These settled particles fall down until they reach the bottom port.

By increasing the liquid inlet flow rate through the bottom port, it is possible to reduce the residence time of liquid inside the inclined settler zones such that smaller particles (e.g., dead cells and cellular debris, or the like) will not have settled by the time the liquid reaches the top of the settling zone, and therefore these smaller particles exit the settler device via a top port. This feature provides a simple method to remove the smaller particles selectively via the top port into a harvest stream, while larger particles (e.g., live and productive cells) are returned from the bottom port to another vessel (such as a bioreactor).

Thus, in these methods, the step of introducing a liquid suspension into these settler devices may include directing a liquid suspension from a plastic bioreactor bag into the particle settler device. Liquid may be directed into, or drawn out of, any ports or openings in the settler device by one or more pumps (for example a peristaltic pump) in liquid communication with the port or opening. Such pumps, or other means causing the liquid to flow into or out of the settler devices, may operate continuously or intermittently. If operated intermittently, during the period when the pump is off, settling of particles or cells occurs while the surrounding fluid is still. This facilitates those particles or cells that have already settled to slide down the inclined sliders unhindered by the upward flow of liquid. Intermittent operation has the advantage that it can improve the speed at which the cells slide downwardly, thereby improving cell viability and productivity. In a specific embodiment, a pump is used to direct a liquid suspension of cells from a bioreactor or fermentation media into the settler devices of the present disclosure.

One parameter that may be adjusted in these methods of using the settler devices of this disclosure is the liquid flow rate into and out of the settler devices. The liquid flow rate will depend entirely on the particular application of the device and the rate can be varied in order to protect the particles being settled and separated from the clarified liquid. Specifically, the flow rate may need to be adjusted to protect the viability of living cells that may be separated in the settler devices of this disclosure and returned to a cell culture, but the flow rate should also be adjusted to prevent substantial cell or particle build up in the settler devices or clogging of the conduits that transfer liquid into and out of the settler devices.

In these methods, the clarified liquid collected from the settler device may include at least one of biological molecules, organic or inorganic compounds, chemical reactants, and chemical reaction products. The clarified liquid collected from the settler device may include at least one of hydrocarbons, polypeptides, proteins, alcohols, fatty acids, hormones, carbohydrates, antibodies, isoprenoids, biodiesel, and beer. In examples of these methods, the clarified liquid collected from the settler device includes at least one of insulin or its analogs, monoclonal antibodies, growth factors, sub-unit vaccines, viruses, virus-like particles, colony stimulating factors and erythropoietin (EPO).

Each publication or patent cited herein is incorporated herein by reference in its entirety. In addition, it is noted Computational Fluid Dynamic (CFD) simulations have confirmed the flow patterns of fluid through the flow channels of the settler devices of the present disclosure and been used extensively to guide the design of elements of embodiments of the settler devices of the present disclosure.

Many autologous and allogeneic cell therapy manufacturing protocols require gentle separation of cells and particles in a sterile, closed flow-through device. For example, ex vivo expansion of mesenchymal stem cells (MSCs) on microcarrier beads in a suspension bioreactor is followed by an enzymatic detachment of cells after they reach confluence on the beads and subsequent separation of MSCs from the beads. Currently the separation of stem cells (~20 microns) from microcarrier beads (∼500 microns) is carried out by passing the mixture through sterile steel mesh with openings of about 100 microns, causing significant shear damage to stem cells and loss of about 15% of the expanded stem cells harvested through this separation process. Another example is in growth of induced pluripotent stem cells (iPSCs) into cell clusters or organoids. The growth of iPSCs requires daily media exchanges to support their fast growth in small bioreactors, such as T-flasks or shake flasks. However, it is difficult to retain the cell clusters inside the bioreactor during their daily media exchanges. Even with extreme care, significant loss of cell clusters (∼100 microns) is experienced during the slow pipetting of the spent media from the flasks.

In contrast, the settler devices of the present disclosure result in less loss and damage of cells. More specifically, settler devices of the present disclosure can separate dead cells and cell debris (<8 microns) from live cells (>12 microns). The settler devices of the present disclosure can also separate 20-micron cells from 500-micron beads in some embodiments. Further, in some embodiments that incorporate sensors for pH and dissolved oxygen (DO), dissolved CO₂, glucose, lactate, glutamine, ammonia, and temperature, control of these culture parameters inside the settler devices of the present disclosure facilitate the growth of various stem cells (iPSCs, MSCs) inside and eliminate the shear damage to cells, cell clusters and microcarrier beads. The sensors can rapidly detect any contamination by adventitious agents like microorganisms in real time or during regular sampling intervals. In addition, a settler device of the present disclosure can be used for stem cell expansion, differentiation, concentration, and harvest without the need for keeping the settler device in and incubator or transporting the settler device between an incubator or a biosafety cabinet.

In one embodiment, the settler devices of the present disclosure can operate in perfusion bioreactor applications to separate and remove dead cells and cell debris (size <10 microns) from the settler device during continuous removal of spent media and recycle of live and productive cells (12 - 24 microns) back into the settler device. In this manner, the settler device of the present disclosure eliminates two major problems experienced by many stem cell expansion and harvesting protocols: (i) daily open operator manipulation steps in a biosafety cabinet for media exchange operations to maintain cell expansion; and (ii) recurrent loss of stem cells and cell clusters during the spent media removal process. More specifically, in some embodiment, the settler devices of the present disclosure eliminate these two problems because it does not require any open operator manipulation in a biosafety cabinet and no viable stem cells or cell clusters are lost during removal of the spent media.

The settler devices of the present disclosure provide additional benefits, including tabletop control of cell culture parameters of the liquid medium inside the settler device. In contrast, all other commercially available stack of tissue culture flasks needs to be placed inside an incubator. Further, the liquid flowing down gently over the adherent cells growing on the inclined slider surfaces inside the settler devices of the present disclosure perfuse the cells with fresh oxygenated media, whereas the other cell culture systems hold the medium static or unmixed on the adherent cells for about 24 hours.

In any of the settler devices of this disclosure, liquid may be directed into, or drawn out of, any of the ports or openings in the housing of the settling device by one or more pumps (for example a peristaltic pump) in liquid communication with the port or opening. Such pumps, or other means causing the liquid to flow into or out of the settler devices, may operate continuously or intermittently. If operated intermittently, during the period when the pump is off, settling of particles or cells occurs while the surrounding fluid is still. This facilitates those particles or cells that have already settled to slide down the inclined slider surfaces unhindered by the upward flow of liquid. Intermittent operation has the advantage that it can improve the speed at which the cells slide downwardly, thereby improving cell viability and productivity. In a specific embodiment, a pump is used to direct a liquid suspension of cells from a bioreactor or fermentation media into the settler devices of the present disclosure.

Spent media can be removed continuously from the settler devices of the present disclosure by pumping fresh media in through other ports. This prevents the loss of any live cells or cell clusters during the removal of the spent media. All other scalable adherent cell culture stacks have to be carried into a biosafety cabinet for daily manual media exchange operations inside.

Finally, harvesting of expanded stem cells after they have grown to confluence on the available growth surface is easily accomplished by adding the required enzyme solutions into the settler devices of the present disclosure and facilitating the detached cells to slide gently down the inclined sliders and exit the settler devices. In contrast, the other adherent cell culture systems have to be taken inside the biosafety cabinet and manipulated extensively to harvest the detached stem cells.

Size distribution data for samples obtained from tests of the settler devices of embodiments of the present disclosure provided about 50% viable cells showing a small peak of dead cells (8 -10 microns) and the settler’s top effluent showing very clearly that the smaller dead cells and cell debris (less than 8 microns) are removed preferentially in the top effluent. Further, viability percentage of cells recovers from its typical drop over 7 days of fed-batch culture to around 90% in our perfusion bioreactor attached with a settler device of the present disclosure soon after the perfusion flow is turned on and increased gradually to remove dead cells and cell debris selectively from the bioreactor.

The settler devices of the present disclosure can be operated as an integrated bioreactor/settler which beneficially replaces two separate devices and eliminates many peristaltic pumps which were previously required for the transport of cell culture liquid between the two current devices. The settler devices can therefore be used for several important applications in cell therapy manufacturing, such as but not limited to: (i) separating single stem cells gently from microcarrier beads, (ii) retaining cell clusters completely while removing spent media continuously in a perfusion operation, (iii) concentrating and harvesting stem cells without any shear damage and (iv) growing adherent stem cells on the large area of inclined settling surfaces inside by installing sensors inside settler device to measure pH, DO, dissolved CO₂, glucose, lactate, glutamine, ammonia, and T and controlling these culture parameters by sparging a manipulated mixture of air, O₂, CO₂, N₂ and air and/or by manipulating the flow rates of different liquid media components pumped into the settler devices.

The settler devices of the present disclosure incorporated inside bioreactors may be advantageous for extracting toxic metabolites and allow yeast cells (e.g., yeast Saccharomyces cerevisiae) to grow further and produce more product. Agitation in the settler devices and bioreactors may cause an oil-water emulsion to form within the housing, which may be broken down to allow for the continual harvesting of the organic phase with a formed toxic product. To assist in the breaking down of the emulsion, the aqueous phase (e.g., a fermentation broth containing the yeast cells) is returned to the bioreactor for further growth and production in a continuous perfusion process. The positioning of the sliders as described throughout the present disclosure would allow for a combination of (1) yeast fermentation in the middle of the settler devices and bioreactors, and (2) emulsion breakage or separation in the sliders about the periphery of the settler devices and bioreactors as described throughout the present disclosure.

The settler devices of the present disclosure inside the bioreactors may be advantageous for separating and harvesting a lighter aqueous phase with extracted product continuously from a periphery within the housing of the settler devices and bioreactor, while also preparing a central agitated fermentation zone to enable cell growth and extraction of a secreted product into the lighter phase for separation in the periphery settling zone.

The settler device provides many advantages over the current state-of-the-art, including the elimination of any need for (i) keeping the settler device inside an incubator to control all the culture parameters within the settler device, (ii) keeping the settler device inside a biosafety cabinet for sterile liquid handling and cell harvesting, and (iii) transporting the settler device back and forth between an incubator and biosafety cabinet for daily media exchanges.

It is contemplated the example embodiments of the bioreactors 600, 700, 800, 1300 in FIGS. 15-34 and 54-55 which represent portions of commercially-available products including the Ambr® 250 microcarrier and/or mammalian vessel manufactured and/or marketed by Sartorius, the BioBLU® single-use bioreactor manufactured and/or marketed by Eppendorf, the KLF bioreactor manufactured and/or marketed by Bioengineering AG, the BioFlo® / CelliGen® line of bioreactors manufactured and/or marketed by Eppendorf, and the Vertical-Wheel® bioreactor manufactured and/or marketed by PBS Biotech®, provide support that the sliders as described throughout the present disclosure should be understood to be configurable with any existing or commercially-available bioreactor or similar product without departing from the scope of the present disclosure.

To provide additional background, context, and to further satisfy the written description requirements of 35 U.S.C. § 112, the following are incorporated by reference herein in their entireties: European Patent EP0521583B1, U.S. Pat. 1,701,068, U.S. Pat. 2,230,386, U.S. Pat. 2,261,101, U.S. Pat. 2,307,154, U.S. Pat. 2,651,415, U.S. Pat. 5,624,580, U.S. Pat. 5,840,198, U.S. Pat. 5,948,271, U.S. Pat. 6,146,891, U.S. Pat. App. Pub. 2005/0194316, U.S. Pat. App. Pub. 2007/0246431, U.S. Pat. App. Pub. 2009/159523, U.S. Pat. App. Pub. 2011/097800, U.S. Pat. App. Pub. 2012/180662, U.S. Pat. App. Pub. 2014/011270, U.S. Pat. App. Pub. 2014/0225286, and U.S. Pat. App. Pub. 2017,0090490.

The foregoing examples of the present disclosure have been presented for purposes of illustration and description. These examples are not intended to limit the disclosure to the form disclosed herein. Consequently, variations and modifications commensurate with the teachings of the description of the disclosure, and the skill or knowledge of the relevant art, are within the scope of the present disclosure. The specific embodiments described in the examples provided herein are intended to further explain the best mode known for practicing the disclosure and to enable others skilled in the art to utilize the disclosure in such, or other, embodiments and with various modifications required by the particular applications or uses of the present disclosure. It is intended that the appended claims be construed to include alternative embodiments to the extent permitted by the prior art.

What is claimed is:

1. A settling device operable to be provided inside a bioreactor for use in the production of cell therapy products, secreted biological proteins, polypeptides or hormones, vaccines, viral vectors, or gene therapy products, comprising:
a housing with a lower end, an upper end, and an outer wall;
an inner structure positioned within the housing, the inner structure including an inner wall, wherein the inner structure and the housing are aligned along a longitudinal axis defined by the housing;
a plurality of inclined sliders provided within a cavity defined by an interior surface of the outer wall and an exterior surface of the inner wall.

2. The settling device of claim 1, each inclined slider of the plurality of inclined sliders comprising a body with a first arcuate surface having a groove and sides, the first arcuate surface spaced from a first adjacent inclined slider, at least one of an edge coupled to the interior surface of the housing or an edge coupled to the exterior surface of the inner structure, and a second arcuate surface spaced from a second adjacent inclined slider.

3. The settling device of claim 1, each inclined slider of the plurality of inclined sliders comprising a body with a first arcuate surface having a plurality of grooves and corresponding sides, the first arcuate surface spaced from a first adjacent inclined slider, at least one of an edge coupled to the interior surface of the housing or an edge coupled to the exterior surface of the inner structure, and a second arcuate surface spaced from a second adjacent inclined slider.

4. The settling device of claim 1, each inclined slider of the plurality of inclined sliders comprising a ring slider including a body with a substantially circular cross-section and including an inclined surface spaced from an adjacent ring slider, wherein the inclined surface is inclined downward toward the lower end of the body of the housing or is inclined upward toward the upper end of the body of the housing.

5. The settling device of claim 1, further comprising at least one of:
an upper section coupled to the upper end of the housing; and
a lower section, where the lower end of the housing contacts and extends upwardly from the lower section.

6. The settling device of claim 5, wherein the lower section further comprises a first port, and the upper section comprises at least a second port extending from the upper section and downwardly in an annular space between the interior surface of the housing and the exterior surface of the inner structure.

7. The settling device of claim 6, wherein one or more of the first port or the at least a second port are oriented approximately parallel to the longitudinal axis.

8. The settling device of claim 6, further comprising a sensor associated with the first port or the at least a second port to measure a condition within the housing, wherein the sensor is operable to measure at least one of pH, dissolved oxygen (DO), dissolved CO₂, glucose, lactate, glutamine, ammonia, and temperature.

9. The settling device of claim 8, wherein the sensor is positioned between the upper and lower ends of the housing.

10. The settling device of claim 1, wherein the inner structure is a first inner structure and the plurality of inclined sliders is a first plurality of inclined sliders, the settling device further comprising:
a second inner structure positioned within the first inner structure, the second inner structure including a second wall, wherein the first inner structure and the second inner structure are concentrically aligned along a longitudinal axis; and
a second plurality of inclined sliders provided within an inner cavity defined by an interior surface of the first inner structure and an exterior surface of the second wall.

11. The settling device of claim 10, each inclined slider of the second plurality of inclined sliders comprising a body with a first arcuate surface having a groove and sides or comprising a body with a first arcuate surface having a plurality of grooves and corresponding sides.

12. A perfusion bioreactor operable for use in the production of cell therapy products, secreted biological proteins, polypeptides or hormones, vaccines, viral vectors, or gene therapy products, comprising:
a housing with a lower end, an upper end, and an outer wall; and
a plurality of inclined sliders provided within a cavity defined at least by an interior surface of the outer wall.

13. The perfusion bioreactor of claim 12, further comprising:
an impeller positioned in the cavity, the impeller including one or more sets of blades,
the inner structure including an inner wall, wherein the inner structure and the housing are concentrically aligned along a longitudinal axis defined by the housing,
the cavity defined by the interior surface of the outer wall and an exterior surface of the inner wall.

14. The perfusion bioreactor of claim 13, the cavity being an outer cavity, the plurality of inclined sliders being positioned in the outer cavity, the inner structure being hollow and defining an inner cavity, the impeller being positioned within the inner cavity, the inner structure including one or more apertures to facilitate fluid flow from the inner cavity to the outer cavity.

15. The perfusion bioreactor of claim 13, the inner structure including a separation plate, the separation plate defining a first cavity portion and a second cavity portion of the cavity, the plurality of inclined sliders being positioned in the first cavity portion and the impeller being positioned in the second cavity portion, the separation plate including an indentation for at least one of the one or more sets of blades of the impeller.

16. The perfusion bioreactor of claim 12, each slider of the plurality of sliders comprising a cone slider including a body with an inclined surface spaced from an adjacent cone slider via one or more projections from the inclined surface.

17. A method of settling particles or cells in a suspension, comprising:
introducing a liquid suspension of particles or cells into a settling device which includes:
a housing with a lower end, an upper end, and an outer wall;
an inner structure positioned within the housing, the inner structure including an inner wall, wherein the inner structure and the housing are concentrically aligned along a longitudinal axis defined by the housing;
a plurality of inclined sliders provided within a cavity defined by an interior surface of the outer wall and an exterior surface of the inner wall; and
a sensor;
measuring one or more of pH, dissolved oxygen (DO), dissolved CO₂, glucose, lactate, glutamine, ammonia, and temperature in the cavity with the sensor;
collecting a clarified liquid from the at least a second port; and
collecting a concentrated liquid suspension from the first port.

18. The method of claim 17, wherein the liquid suspension comprises at least one of:
a recombinant cell suspension, an alcoholic fermentation, a suspension of solid catalyst particles, a municipal wastewater, industrial wastewater, mammalian cells, bacterial cells, yeast cells, plant cells, algae cells, plant cells, mammalian cells, murine hybridoma cells, stem cells, CAR-T cells, red blood precursor and mature cells, cardiomyocytes, yeast in beer, and eukaryotic cells;
recombinant microbial cells selected from at least one of Pichia pastoris, Saccharomyces cerevisiae, Kluyveromyces lactis, Aspergillus niger, Escherichia coli, and Bacillus subtilis; and
one or more of microcarrier beads, affinity ligands, and surface activated microspherical beads; and
wherein the clarified liquid collected comprises at least one of biological molecules, organic or inorganic compounds, chemical reactants, chemical reaction products, hydrocarbons (e.g., terpenes, isoprenoids, polyprenoids), polypeptides, proteins (e.g., brazzein, colony stimulating factors), alcohols, fatty acids, hormones (e.g., insulin, growth factors), carbohydrates, glycoproteins (e.g., erythropoietin, monoclonal antibodies), beer, and biodiesel.

19. The method of claim 17, further comprising controlling at least one of pH, dissolved oxygen, dissolved CO₂, glucose, lactate, glutamine, and ammonia within the settling device by manipulating the flow rates of at least one of air, O₂, CO₂ and N₂ introduced into the settling device or manipulating the flow rates of different liquid media components.

20. The method of claim 17, wherein introducing a liquid suspension of particles into the settling device comprises pumping the liquid suspension through at least a third port located in the upper section.
